(19) United States
(12) Patent Application Publication
(10) Pub. No.: US 2011/0041191 A1
(43) Pub. Date: Feb. 17, 2011

(54) Animal model, and products and methods useful for the production thereof

(76) Inventors: Bettina Platt, Aberdeen (GB); Gernot Riedel, Aberdeen (GB)

Correspondence Address: MICHAEL BEST & FRIEDRICH LLP, 100 East Wisconsin Avenue, Suite 3300, Milwaukee, WI, 53202, US

(21) Appl. No.: 12/833,474

(22) Filed: Jul. 9, 2010

Related U.S. Application Data
(60) Provisional application No. 61/224,137, filed on Jul. 9, 2009.

(51) Int. Cl. A01K 67/027; C12N 5/10; C12N 15/63; C07H 21/04; G01N 33/48; C12N 15/89

(52) U.S. Cl. 800/3; 435/352; 435/320.1; 536/23.5; 800/12; 800/14; 800/18; 800/21

(57) ABSTRACT

The present invention relates to a transgenic animal suitable for modelling Alzheimer's Disease. The present invention also relates to cells and gametes of the transgenic animal of the invention, along with nucleic acids and vectors suitable for generating the transgenic animal. Methods of generating the transgenic animal are also described, along with screening methods utilizing the transgenic animal.

### TECHNICAL FIELD

The present invention relates generally to transgenic rodents, particularly mice, expressing an APP and/or a tau polynucleotide sequence and showing Alzheimer's disease related phenotypes.

### BACKGROUND ART

Alzheimer's disease (AD), the most common form of dementia in the elderly, is characterised by a progressive decline of cognitive abilities, and histological hallmarks associated with increased amyloid levels in form of extracellular plaques, and soluble, non-fibrillary amyloid species (Haass & Selkoe, 2007; Selkoe, 2001). Moreover, neurofibrillary degeneration occurs, which is caused by abnormal phosphorylation of tau, a microtubule-associated protein (Grundke-Iqbal et al, 1986, Iqbal et al., 2009). Gross morphological atrophy is manifested across a number of forebrain structures, including hippocampus and cortex (Vickers et al., 2000).

A number of genotype to phenotype relationships for genetic mutations linked to familial cases of AD (fAD) has enhanced the understanding of the disorder. However, even though tau pathology consistently correlates with cognitive impairments while plaque load does not (Giannakopoulos et al., 2007), a direct genetic link for the tau gene has only been identified in other tauopathies, such as fronto-temporal dementia (FTDP). Furthermore, the vast majority of AD cases are idiopathic (˜>90%), with no obvious genetic link. Primary and secondary causes and parameters affecting onset and progression of AD are thus yet to be fully elucidated.

### Existing Models & tau vs Amyloid Hypothesis

AD is a human-specific condition with most hallmarks absent in other species. Research relies on animal models of AD that should ideally recapitulate molecular, cellular, and cognitive changes and show an age-dependent progression. Such models are vital for the characterisation of underlying mechanisms, the identification of therapeutic targets and the development of potential treatments prior to human clinical trials.

To date, numerous pharmacological and genetic models exist (Woodruff-Pack, 2008; McGowan et al., 2006), the majority of which are based on rodents. Genetic models incorporate key genes identified in fAD, such as human variants of the amyloid precursor protein (APP), presenilins, as well as tau genes implicated in other tauopathies. Advantages of using mice include similarities to humans in CNS morphology and physiology, the high rate of fecundity, the ease with which they can be manipulated genetically and the ability to conduct cognitive testing (Hofker, 2002). Conversely, their short life-span, as such a desirable parameter, makes age studies difficult, and they appear to be resistant to some degenerative processes (e.g. plaque formation).

The most commonly utilised and best characterised mouse transgenic models (e.g. PDAPP, Tg2576, P301L, 3×LaFerla mouse models) present with a variable range of behavioural, biochemical, pathological and physiological traits simulating AD. First generation transgenic AD mouse models were based on the introduction of full length or mutated human APP, PSEN or tau genes, crossing of the resultant species led to second-generation double and triple mutants. More recently, Oddo and colleagues generated a triple transgenic mouse model (3×AD model), by dual pro-nuclear injection of APP_{swe} and the P301L tau gene constructs, each under the control of a Thy1.2 promoter, into single-cell embryos from a PSEN knock-in line. In this model, cognitive deficits are observed before the onset of overt amyloid accumulation, which in turn precedes neurofibrillary pathology (see papers by Oddo and LaFerla).

While advancements have been made in our understanding of AD, it is of interest to note that a full understanding is still elusive, and data obtained in the existing models have as yet not led to a major break-through or development of treatment strategies.

Problems associated with models developed thus far are their inability to fully recapitulate the entire spectrum of hallmarks, and, more importantly the hugely variable link between histo-pathology and cognitive performance. Interestingly, cellular and cognitive deficits are often observed ahead of amyloid and tau pathology, suggesting intraneuronal or soluble bA oligomers as a cause (e.g. Haass & Selkoe, 2007; Smith et al., 2005; Billings et al., 2005). Many models are not region- and cell-type specific, and show a number of non-AD phenotypes such as motor impairments, often due to promoters used to boost high transgene expression. Additionally, pronuclear injection procedures utilised to generate tg animals, have resulted in a number of potential pitfalls (Deng and Siddique, 2000). Random integration of transgenes into unknown and often instable insertion sites, and in unpredictable copy numbers (Palmiter and Brinster, 1986) result in variable phenotypes and can lead to a loss of pathology over generations. The site of integration also affects expression of the transgene, and disruption of the endogenous function may contribute to the phenotype, a problem compounded when the transgenic mice are bred to homozygosity (Meisler, 1992).

In general, models thus far have aimed for aggressive, early onset phenotypes, with high tg expression, often during early development (Smith et al., 2005, Oakley et al., 2006), leading to extreme phenotypes of doubtful relevance (e.g. Gotz et al., 2000), since both APP and tau play a number of potentially important roles in development (Sheng et al., 2006). Compensation (both genetically and physiologically) is likely to occur in such models, and cannot be controlled for.

Therefore, the validity, reproducibility, reliability and consistency of phenotypes should take precedence over cost- and time-effectiveness.

### Endpoints and Bio-Markers

A wide range of criteria are employed to validate transgenic models. Procedures and endpoints combine by and large histological (anatomical & neurochemical), physiological, molecular and behavioural parameters. Amyloid and tau related immunocytochemical and molecular studies are currently essential for the assessment of disease severity and progression, and a large number of tools are available. However, many antibodies show species cross-reactivity, standard tissue ELISAs do not exist for tau pathology, while amyloid ELISAs do not detect levels <pg/ml.

Much effort has been made to develop suitable behavioural testing that allows investigation of different memory systems (e.g. short vs long-term, declarative vs procedural) relevant to humans, since protection and recovery of cognitive abilities is an important goal. Most popular paradigms are maze and recognition/discrimination paradigms. Additionally, some attempts have been made to assess general activity and emotional aspects in AD models, as potential non-invasive, disease relevant biomarker [e.g. Knobloch et al., 2007]. Synaptic transmission and plasticity, studied commonly by lectrophysiological means in hippocampal slices, is also considered an essential experimental endpoint, and has proven to be most sensitive in many AD models [Platt et al. and others]. In vivo electrophysiology, and surface EEG recordings in particular, has also recently experienced a revival due to technical advancements and computational tools, and may offer promise as a translational procedure. Another non-invasive procedure, established in humans and being currently developed for animals is brain imaging.

Overall, the existing hyper-expression models have led to some insight into disease mechanisms, yet, unspecific and variable phenotypes are commonly reported, and hallmarks and progression are not fully mimicked in any of these models. Thus, ground breaking progress requires improved experimental models combined with innovative surrogate biomarkers of disease onset and progression (see comments in Nature, 454: 682-685 & 456: 161-164; 2008).

### DISCLOSURE OF THE INVENTION

The present inventors have devised a new and improved animal model for Alzheimer's disease. The transgenic rodents described herein, and the related methods and uses, are intended to address one or more problems associated with existing transgenic Alzheimer models.

The novel animal model is based on the targeted ‘knock-in’ of a mutated APP (amyloid precursor protein) gene together with a mutated tau gene into the genome of a rodent at a single locus, using a targeting vector. Preferably, the transgenes are present in the transgenic rodent in a single copy per cell. The mutated transgenes are expressed in the transgenic rodent as mutated polypeptides, causing or contributing to the observed phenotypes. Though the described animal model presents with a more subtle, phenotype compared to some of the existing models, in preferred embodiments the present invention has several advantages over the existing models.

First, the products and methods described herein can be used to generate single, double or triple knock-in transgenic rodents of high consistency and comparability. First there is provided an APP/Tau double knock-in transgenic rodent. The transgenes are contiguously arranged at a single genetic locus. Each transgene is flanked by a set of excision sequences. Single knock-in transgenic rodents can be generated by specifically excising one of the two transgenes of the APP/Tau double knock-in transgenic rodent. Triple knock-in transgenic rodents can be generated by introducing a further transgene (such as for example presenilin or a mutated form thereof) into the APP/Tau double knock-in transgenic rodent. As the methods utilize the same APP/Tau double knock-in rodent, either by deleting one of the transgenes or by adding a further transgene, the resulting strains are directly comparable and highly informative regarding the contribution of the individual genes. Generated by blastocyst injection, the APP/Tau double knock-in and any single or triple knock-in generated therewith are thus based on the same targeted embryonic stem cell. The triple knock-in rodents may be used to generate further double knock-in animals by excising either the APP or tau transgene.

Secondly, it allows for high consistency and reproducibility. This will not only allow better comparison of results obtained using different procedures, but will also enable more cost- and time effective studies (e.g. fewer repeat controls needed and lower n's per group). Further, due to the targeted insertion into the genome, any impact from positional effects and possible instable insertion sides can be excluded. Alterations in the phenotype and biomarkers are due to the expression of the transgenes and not due to any interference with the endogenous genome. Variation in the copy number is also excluded.

Further, thorough sensory motor testing and health screening show no overt disease-unrelated phenotypes in the transgenic rodents (e.g. normal growth and no motor deficits, as for instance is seen in many previous tau models).

The transgenic rodents described herein provide a combination of disease relevant phenotypes including a series of novel features not previously reported.

The present invention thus relates to transgenic rodents, expressing mutated APP and/or mutated tau polynucleotide sequence, as well as methods and uses thereof. It further relates to nucleic acids, vectors and cells useful in the generation of the transgenic rodents. It further relates to methods of generating the transgenic rodents, as well as their use. In particular, they relate to screening methods and methods for modelling Alzheimer's disease.

Thus, in one aspect of the invention there is provides a transgenic rodent which includes within a plurality of its cells a nucleic acid comprising (1) a mutated APP polynucleotide sequence flanked by a first set of excision sequences, and (2) a mutated tau polynucleotide sequence flanked by a second set of excision sequences, wherein (1) and (2) are operably linked to the same promoter sequence at a single locus. The promoter sequence may be a CamKII sequence. The polynucleotides may be heterologous with respect to the transgenic rodent.

The transgenic rodent may be hemizygous, heterozygous or homozygous with respect to the nucleic acid

The excisions sequences may comprise or consist of IoxP or FRT sequences. By using a different set of excision sequences for each transgene, each transgene can be specifically excised using the respective recombinase.

In some embodiments the mutated APP polynucleotide sequence encodes a polypeptide comprising one or more of the following mutations: K670N; M671L; and V717I. In some embodiments the mutated tau polynucleotide sequence encodes a polypeptide comprising one or more of the following mutations: P301L and R406W.

In some embodiments the mutated APP polynucleotide sequence comprises SEQ ID NO:1. In some embodiments the mutated tau polynucleotide sequence comprises SEQ ID NO:2.

The nucleic acid may further comprise a marker gene, such as for example the marker neomycin resistance gene. Presence of the marker gene allows selection for the presence of the nucleic acid. The nucleic acids may further comprise an internal ribosome entry site positioned between the APP and tau polynucleotide sequences.

In some embodiments, the single locus is the HPRT locus.

In some embodiments, the nucleic acid is present in the transgenic rodent at one copy per cell.

In some embodiments, the transgenic rodent includes within said plurality of cells a presenilin polynucleotide sequence. The presenilin sequences may be wildtype presenilin sequences, or mutated forms. For example, it may be PSNE1.

In some embodiments, the transgenic rodent may have one or more of the following phenotypes: intracellular and extracellular amyloid deposits, impaired synaptic transmission, reduced paired pulse facilitation (PPF), deficit in LTP, reduced activity in dark phase, spending more time awake, sleep disturbance and sleep fragmentation, reduced REM and NREM sleep, cognitive deficits, altered memory, premature aging, and altered metabolism in the brain.

In one aspect, the invention provides a somatic cell or tissue sample of the transgenic rodent described herein. In one aspect, the invention provides a gamete of the transgenic rodent as described herein.

In one aspect, the invention provides a nucleic acid comprising a (1) a mutated APP polynucleotide sequence flanked by a first set of excision sequences, and (2) a mutated tau polynucleotide sequence flanked by a second set of excision sequences, wherein (1) and (2) are operably linked to the same promoter sequence.

The first set of excision sequences may comprise loxP sequences or FRT sequences. The second set of excision sequences may comprise loxP sequences or FRT sequences, wherein preferably the first and second set of excision sequences are different from each other.

In some embodiments, the mutated APP polynucleotide sequence encodes a polypeptide comprising one or more of the following mutations: K670N; M671L; and V717I. In some embodiments, the mutated tau polynucleotide sequence encodes a polypeptide comprising one or more of the following mutations: P301L and R406W. In some embodiments, the mutated APP polynucleotide sequence comprises SEQ ID NO:1. In some embodiments, the mutated tau polynucleotide sequence comprises SEQ ID NO:2.

In some embodiments, the promoter sequence is a CamK2 promoter. In some embodiments, the nucleic acids comprises a marker gene, such as the neomycin resistance gene. In some embodiments, the nucleic acid comprises an internal ribosome entry site positioned between the APP and tau polynucleotide sequences.

In one aspect, the invention provides a vector comprising the nucleic acid described herein.

In one aspect, the invention provides a targeting vector comprising the nucleic acid described herein, and further comprising a targeting sequence. The targeting sequence may be a sequence targeting the HPRT locus.

In one aspect, the invention provides a cell comprising the nucleic acid described herein. The cell may be an embryonic stem cell.

In one aspect, the invention provides a method of generating a transgenic rodent, the method comprising
(a) injecting an ES cell into a rodent blastocyst, the ES cell comprising the nucleic acid described herein,
(b) implanting said blastocyst into a surrogate female rodent,
(c) allowing the surrogate female rodent to produce offspring, and
(d) screening the offspring for the introduction of said nucleic acid in the genome.

The method may further comprise the step of crossing the offspring with a wildtype rodent of the same species and obtaining F1 offspring.

In some embodiments, the method may further comprise the steps of

(i) providing the offspring of any one of the methods described herein,
(ii) excising the APP or tau polynucleotide sequence, and optionally (iii) obtaining resulting offspring after step (ii) and optionally (iv) testing the resulting offspring for the excision of the APP or tau polynucleotide sequence, respectively.

In some embodiments, the method may further comprise the steps of

(i) providing the offspring of any one of the methods described herein,
(ii) crossing the offspring with a rodent capable of expressing a recombinase specific for the first or second set of recombination sites. The method may further comprise the steps of (iii) obtaining the resulting offspring, and optionally (iii) testing the resulting offspring for the excision of the APP or tau polynucleotide sequence, respectively.

In some embodiments, the methods described above may further comprise the step of (i) crossing the F1 offspring or resulting offspring with another transgenic rodent, said other transgenic rodent including a mutant presenilin polynucleotide sequence. The method may further comprise the steps of (ii) obtaining offspring, and optionally (iii) testing the offspring of step (ii) for the presence of one or more of said APP, tau and presenilin polynucleotide sequences.

In one aspect, the invention provides a transgenic rodent obtainable by any one of the methods described above.

In one aspect, the invention provides a method of modelling Alzheimer's disease by providing the transgenic rodent described herein and monitoring changes in one or more of the phenotypes of the rodent.

In one aspect the invention provides a method of screening or assessing a compound suspected of having a therapeutic effect in relation to Alzheimer's disease, the method comprising: (a) providing the transgenic rodent describe herein, (b) administering the compound to the rodent, (c) monitoring changes in one or more of the phenotypes of the rodent. The phenotype monitored may be selected from intracellular and extracellular amyloid deposits, impaired synaptic transmission, reduced paired pulse facilitation (PPF), deficit in LTP, reduced activity in dark phase, spending more time awake, sleep disturbance and sleep fragmentation, reduced REM and NREM sleep, cognitive deficits, altered memory, premature aging, and altered metabolism in the brain. In one aspect, the invention provides a transgenic rodent, cell, gamete, or method as described herein, wherein the transgenic rodent is a mouse.

In one aspect, the invention provides a system comprising

(1) providing a double or triple transgenic rodent generated by any one of the methods described herein,
(2) providing an excised control rodent obtainable by the methods described herein,
(3) comparing the phenotype of (1) with the phenotype of (2).

Some of these aspects will now be described in more detail.

In a first aspect, the invention provides a transgenic rodent which includes within a plurality of its cells a nucleic acid comprising (1) a first disease-related polynucleotide sequence flanked by a first set of excision sequences, and (2) a second disease-related polynucleotide sequence flanked by a second set of excision sequences, wherein (1) and (2) are operably linked to the same promoter sequence at a single genetic locus. The disease-related polynucleotide sequences are preferably Alzheimer's disease related sequences.

A polynucleotide sequence is considered a disease-related sequence if, for example, any alteration of its polynucleotide sequence or of the encoded protein, its under- or overexpression or dysregulation in an organism or cell, its introduction in a cell or organism or any other kind of interference with said polynucleotide or the encoded protein (for example modification of any functional properties or effect normally observed with said sequence) leads to, contributes or causes effect which are associated with a particular disease. Of particular interest are Alzheimer-disease related sequences.

Examples of Alzheimer's related sequences are APP, tau and presenilins, either in wildtype or mutated form. While certain aspect of the invention are described with reference to APP, tau or presenilin, it is understood that further disease-related polynucleotide sequences could be used in the methods and products described herein.

In some embodiments, the first disease-related polynucleotide sequence is selected from APP and tau. In some embodiments, the second disease-related polynucleotide sequence is selected from APP and tau, wherein the first and second disease-related polynucleotide sequences are not identical.

In preferred embodiments, the first or second disease-related polynucleotide sequence is APP. In preferred embodiments, the first or second disease-related polynucleotide sequence is tau.

Thus, in a further aspect, the invention provides a transgenic rodent which includes within a plurality of its cells a nucleic acid comprising (1) a (mutated or non-mutated) heterologous APP polynucleotide sequence flanked by a first set of excision sequences, and (2) a (mutated or non-mutated) heterologous tau polynucleotide sequence flanked by a second set of excision sequences, wherein (1) and (2) are operably linked to the same promoter sequence at a single genetic locus. In some embodiments, the transgenic rodent may further comprise a third transgene, such as for example a mutated or non-mutated form of presenilin. Thus, in some embodiments the present invention discloses PLB1 triple knock-in mice, which carry a mutated APP, tau and presenilin transgene (with APP and tau being located at a single genetic locus).

In some embodiments either the mutated APP or mutated tau polynucleotide sequence (or both) is excised from the genome of the transgenic rodent, generating a single knock-in (or a non-mutant) line. The non-mutant line can be helpful to assess the influence of the remaining construct components in the genome, such as for example the promoter sequence, or if present the selectable marker gene.

In some embodiments the single genetic locus is a locus on the X chromosome, preferably the locus for the hypoxanthine phosphoribosyltransferase (HPRT) gene. Since the HPRT locus is located on the X chromosome, animals generated by mating heterozygous females with transgenic (hemizygous) males are either heterozygous or homozygous females, hemizygous males or wild type (WT) males.

Using a targeting vector, the inventors were able to insert the APP and tau transgenes in a controlled and specific way at a predetermined locus, avoiding negative impact of the endogenous genome by gene interruption or position effect of endogenous regulatory sequences. The targeted knock-in procedure thus allowed for stable and consistent gene expression. It also allowed to control the copy number present in each cell. In preferred embodiments, the HPRT™ targeting vector is used to insert the ATT and tau transgenes at the HPRT locus. Preferably, the heterologous nucleic acid is present at one copy per cell.

The transgenes are flanked by different sets of excision sequences. For example, APP may be flanked by loxP sites (IoxP sequences) and tau by Frt sites (Frt sequences), or vice versa. Other excision systems may be used. The presence of two distinct set of excision sequences allow the selective deletion of either the tau or the APP polynucleotide sequence, for example by crossing the double knock-in transgenic rodents with a rodent that either expresses the cre recombination (with respect to IoxP) or the Flippase recombinase (with respect to Frt). This allows the generation of single transgenic models from the same targeted embryonic stem cell which in turn allows a precise comparison of the effects of each gene or combination.

In a further aspect the invention provides systems and method for assessing and/or evaluating the role of a polynucleotide sequence (or polypeptide sequence) in the formation or pathology of Alzheimer's disease.

In one aspect there is provided a system comprising

(1) providing a double or triple transgenic rodent generated by the methods described herein,
(2) providing an excised control rodent obtainable by the methods described herein,
(3) comparing the phenotype of (1) with the phenotype of (2).

A double transgenic rodent refers to a transgenic rodent carrying two transgenes, a triple transgenic rodent refers to a transgenic rodent carrying three transgenes. As described elsewhere herein, double and triple knock-in transgenic rodents can be generated using the methods herein, wherein the methods utilise the same double knock-in transgenic rodent (tau/app double) and thus are based on the same targeted embryonic stem cell. Using the methods described herein, these double or triple transgenic rodents can be used to generate controls by excising one or more of the transgenes from the rodent. This can for example be done by introducing a recombinase that specifically recognises the excision sequences flanking the transgenes. Individual transgnenes can thus be specifically excised, providing excised controls. An excised control rodent thus differs from the respective double or triple transgenic rodent by the presence or absence of one, two, three transgenes from the double and triple transgenic rodent in question. Because the excised control and the double or triple transgenic rodent are based on the same targeted embryonic stem cell and vary in the presence or absence of the one or more (excised) transgenes, they are directly comparable and highly informative, as any differences observed are caused by the absence or presence of the one or more (excised) transgenes. Ideally, the double/triple transgenic rodent and the respective excised control only vary in the absence or presence of one transgene, so that any effect can be directly correlated to one specific gene. By comparing the phenotypes of both the double or triple transgenic rodent with the phenotype of the control, important information regarding the role and function of the respective gene(s) and their contribution to the formation and pathology of Alzheimer's disease can be gained.

In one aspect there is provided a system comprising

(1) providing a double or triple transgenic rodent as described herein,
(2) providing an excised control rodent wherein the excised control rodent differs from the rodent of (1) with respect to the presence of one transgene,
(3) comparing the phenotype of (1) with thephenotype of (2).

In one aspect there is provided a system comprising

(1) providing a double or triple transgenic rodent as described herein,
(2) providing an excised control rodent wherein the excised control rodent differs from the rodent of (1) with respect to the presence of two transgenes,
(3) comparing the phenotype of (1) with the phenotype of (2).

### Heterologous Nucleic Acid

The term “heterologous” is used broadly in this aspect to indicate that the mutated APP and mutated tau polynucleotide sequences have been introduced into said construct or said cells of the rodent, or an ancestor thereof, using genetic engineering, i.e. by human intervention. Preferably the mutated polynucleotide sequences are human, but they may be derived from any species.

The APP and tau polynucleotide sequences are arranged contiguously in one construct, optionally spaced by a short intervening sequence which may comprise an internal ribosome entry site (IRES) nucleotide sequence to allow translation initiation and protein synthesis of APP and tau proteins from a single mRNA strand. Each polynucleotide sequence is flanked by a set of excision sequences, i.e. sequences that can be recognised by respective recombinases. Using a different set of excision sequences for each polynucleotide sequence allows the specific removal of one of the polynucleotide sequences. As set out above, examples of excision sequences are loxP and FRT sequences.

The mutated APP and mutated tau polynucleotide sequence are both operably linked to the same promoter sequence. Suitable promoter sequences are known in the art. For example, the mouse CamK2 promoter may be used.

In preferred embodiments, the various elements on the construct are arranged in the following order: promoter sequence, first excision sequence of the first set of excision sequences, a first polynucleotide sequence (such as for example APP), a first excision sequence of the second set of excision sequences, an IRES, a second excision sequence of the first set of excision sequences, a second polynucleotide sequence, a second excision sequence from the second set of excision sequences, and optionally a selectable marker gene (such as for example the neomycin resistance gene). Due to the reversed order of one member of each set of excision sequences, this arrangement allows for the removal of the IRES if one or the other polynucleotide is excised using the first or second set of excision sequences.

The term “mutated APP polynucleotide sequence” and “mutated tau polynucleotide sequence” refers to APP and tau polynucleotide sequences that differ from APP and tau wildtype forms. The mutated APP and tau polynucleotide sequences may be mutated forms of the human APP wildtype (isoform 770, Accession number NM_{—}000484) and of the human four-repeat tau (NM_{—}016835). For example, they may be derived from App wildtype (isoform 770, Accession number NM_{—}000484) and from the human four-repeat tau (NM_{—}016835) by modification of the wildtype polynucleotide sequence.

The mutated forms may have been generated by deletion, insertion, modification, substitution of one or more nucleotides or amino acids, or otherwise. The polynucleotide sequences may carry one or more mutations compared to the wildtype sequence. The mutations comprise mutations previously associated with Alzheimer's disease and mutations not previously associated with Alzheimer's diseases.

In some embodiments, the mutated human APP is the human APP (isoform 770, NM_{—}000484) with one or more mutations giving rise to one or more of the following alterations: Swedish (K670N; M671L) and London (V7171). In some embodiments, the mutated human tau polynucleotide sequence is the human four-repeat tau (NM_{—}016835) with one or more mutation giving rise to the P301L and/or R406W alteration.

In some embodiments, the mutated APP polynucleotide sequence comprises or consists of the cDNA derived from the polynucleotide sequence with Accession number NM_{—}000484).

In some embodiments, the mutated tau polynucleotide sequence comprises or consists of the cDNA derived from the polynucleotide sequence with Accession number NM_{—}016835).

In preferred embodiments, the mutated APP polynucleotide sequence comprises or consists of SEQ ID NO:1.

In preferred embodiments, the mutated tau polynucleotide sequence comprises or consists of SEQ ID NO:2.

In preferred embodiments, the mutated APP polynucleotide sequence comprises or consists of SEQ ID NO:1 and the mutated tau polynucleotide sequence comprises or consists of SEQ ID NO:2.

Presenilin sequences such as PSEN1 are known in the art.

The APP and tau wildtype sequences may also be used in the products and methods described herein.

In some embodiments the nucleic acid may comprise a selectable marker gene, such as for example the neomycin resistance gene.

The transgenes are expressed in the transgenic rodents, giving rise to mutated proteins which can be detected by for example immunohistochemistry in tissue samples obtained from the animals.

### Cells and Tissues

The invention further provides a cell or tissue sample of the transgenic rodent as defined above e.g. which comprises:

(1) a mutated APP polynucleotide sequence flanked by a first set of excision sequences, and (2) a mutated tau polynucleotide sequence flanked by a second set of excision sequences, wherein (1) and (2) are operably linked to the same promoter sequence at a single genetic locus.

Thus the invention also provides a neuron or other somatic cells having these properties from the transgenic rodent, for example in culture.

The invention further provides gametes from the transgenic rodent. These may include:

(1) a mutated APP polynucleotide sequence flanked by a first set of excision sequences, and (2) a mutated tau polynucleotide sequence flanked by a second set of excision sequences, wherein (1) and (2) are operably linked to the same promoter sequence at a single genetic locus.

### Nucleic Acid

The invention also provides modified proteins, RNA and DNA derived from, or for use in the characterization and production of, the transgenic rodents described herein.

In one aspect the invention provides a nucleic acid comprising a
(1) a heterologous mutated APP polynucleotide sequence flanked by a first set of excision sequences, and
(2) a heterologous mutated tau polynucleotide sequence flanked by a second set of excision sequences
   wherein (1) and (2) are operably linked to the same promoter sequence.

It will be appreciated that a nucleic acid will be at least partially synthetic in that it will comprise nucleic acid sequences which are not found together in nature but which have been ligated or otherwise combined artificially.

Nucleic acids may comprise, consist or consist essentially of any of the sequences disclosed herein.

Nucleic acid sequences may be provided and utilised by techniques known in the art (for example, see Sambrook, Fritsch and Maniatis, “Molecular Cloning, A Laboratory Manual”, Cold Spring Harbor Laboratory Press, 1989, and Ausubel et al., Short Protocols in Molecular Biology, John Wiley and Sons, 1992) or later editions of the same. These techniques include (i) the use of the polymerase chain reaction (PCR) to amplify samples of the relevant nucleic acid, e.g. from genomic sources, and RNA.

Nucleic acids may be in the form of vectors e.g. plasmids, cosmids, BAC and YAC vectors. In particular, nucleic acids may be in the form of a targeting vector. A targeting vector allows site specific insertion of a nucleic acid at a predetermined genetic position in a genome. This is generally achieved by a sequence in the targeting vector (i.e. a targeting sequence) which is homologous to a sequence at the target locus, allowing homologous recombination to take place at this target locus. A targeting sequence is such a sequence homologous to a sequence present at the targeted locus. An example of a targeting vector is the HPRT™ targeting vector (genOway), but other targeting vectors are known in the art. Random integration into the genome is such avoided, omitting any associated negative impact from the endogenous genome such as gene disruption or positional effects and resulting in stable and controlled expression of the transgene(s).

### Phenotypes of Transgenic Rodents

Using the targeted knock-in technology the inventors have generated a new animal model for Alzheimer's disease.

By way of exemplification, in the Examples below the inventors describe transgenic mice expressing mutated APP and tau polynucleotide sequences in a single genetic locus, the HPRT locus. The inventors also describe triple knock-in mice, which in addition to the APP and tau polynucleotide sequences further comprise a mutated form of the presenilin gene.

The transgenic rodents described herein display an array of Alzheimer's disease related abnormalities, which are discussed below, which make the transgenic rodents described herein valuable tools for the elucidation of Alzheimer's disease and the underlying mechanisms. Thus, in preferred embodiments, the animal models of the invention may display one or more, preferably all, of the following phenotypes.

### Tissue Expression

The transgenes are stably expressed in the transgenic rodent. Histology and gene expression analysis confirm forebrain expression of both APP and Tau.

Intracellular amyloid species are detected primarily within the soma of neurons from 6 months of age in hippocampal and cortical areas, though pronounced staining in the apical dendrites of the CA1 neurons was also found. Independent from age, extracellular amyloid deposits were detected infrequently (<6 per section). For detection of diffuse plaques, β-sheet aggregation can be determined by Congo Red and Thioflavin-S staining. Lack of massive plaque formation is not an issue as this biomarker has proven not to yield a predictive value regarding cognitive function and efficacy of treatment in both humans and previous animal models. No overt less neuronal loss suggests that the deficits observed are not a result of advances degeneration but rather signalling impairment, this is in agreement with features of other transgenic mouse models of AD (Games et al., 1995; Hsiao et al., 1996; Oddo et al., 2003), and current theory of AD as a synaptic disorder in the early stages.

### Synaptic Transmission and Plasticity

Hippocampus specific physiological deficits could be detected using the hippocampal slice preparation, in vivo (EEG) electrophysiology and FDG PET at 5-6 months of age, and in hippocampus-dependent learning and memory tasks at 8-9 months of age. In particular, transgenic mice showed impaired basic synaptic transmission at 12 months of age and, with respect to short-term plasticity, a reduced paired pulse facilitation (PPF). Transgenic mice also showed a significant deficit in Theta-burst LTP.

### Circadian Rhythms, Vigilance States and Global Brain Activity (EEG)

Circadian locomotor activity was determined in the PhenoTyper system. Transgenic animals exhibit lower activity compared to age-matched wildypes during the dark phase. Analyses of EEG and activity guided vigilance state classification during the light phase suggests that the transgenic animals have a significant genotype effect in all stages. They spend significantly more time awake compared to wildtype. They also show sleep disturbance and sleep fragmentation. Transgenic animals show reduced or disturbed REM and NREM sleep. Overall, the PFx EEG delta range during wakefulness and REM appears to be an indicator of early genotype-specific changes (at 5 months), while severe genotype-specific changes are identified at 13 months in NREM spectra of PFx (theta and gamma) and RH (delta & theta). Table 1 and 2 summarise genotype effects and interactions.

### Behavioural Changes

Age- and genotype specific changes were uncovered regarding differences between transgenic and wild types in circadian activity, sleep pattern and habituation to a novel environment. Sleep fragmentation is pronounced at 13 months of age, also strongly reflected in altered EEG activity during NREM sleep.

Learning and memory: The transgenic animals show changes in learning and memory, in particular with respect to object recognition (tested with object recognition paradigm) and social interaction and recognition (social recognition task, 2 way ANOVA). For example, the PBL1 triples show reduced interest to explore new objects. They also present with reduced interest in social stimuli, and do not show memory for a familiar compared to a novel stranger from 9 months of age.

The transgenic rodents of the invention show altered memory activity. Alterations in memory were detected in PLB1 triples in an object recognition and social (olfactory) recognition paradigm from 8-9 months of age.

### Evidence for Premature Ageing

The transgenic rodents of the invention further show premature aging. Comparison of age profiles in WT vs. Triples suggest that the latter have prematurely aged. This is supported by data from FDG PET, EEG parameters, general behaviour/activity and memory performance.

### PET (Positron Emission Tomography)

At 5 months, PBL1 triple mice showed large areas of decreased metabolism in the forebrain (hippocampal regions and adjunct limbic structures), and some additional areas in the dorsal midbrain and brainstem. At 15 months, they showed a wide-ranging increase in metabolism, with only some dorsal cortical areas showing reduced metabolism. Thus, it seems that PLB1 triple mice are prematurely aged at 5 months compared to wildtype, followed by increased metabolic activity as means to compensate for progressive deficit.

The transgenic rodents of the present invention may display one or more of the phenotypes described herein. In particular, transgenic rodents as described herein may have the following phenotypes compared to wild type rodent: (moderate) intracellular and extracellular amyloid deposits, impaired basic synaptic transmission, reduced paired pulse facilitation (PPF), deficit in Theta-burst LTP, less activity in dark phase, spending more time awake, sleep disturbance and sleep fragmentation such as reduced REM and NREM sleep, cognitive deficits such as altered memory, premature aging, decreased metabolism in the brain measured by PET—in particular in the forebrain (hippocampal regions and adjunct limbic structures) as well as areas in the dorsal midbrain and brainstem at the age of 5 months, and dorsal cortical areas at 15 months; increased metabolism in the brain—in particular at the age of 15 months.

### Methods of Generating Transgenic Rodents

In a further aspect, there is provided a method of generating a transgenic rodent, the method comprising
(a) injecting an ES cell into a rodent blastocyst, the ES cell comprising the nucleic acid described herein,
(b) implanting said blastocyst into a surrogate female rodent,
(c) allowing the surrogate female rodent to produce offspring, and
(d) screening the offspring for the introduction of said nucleic acid in the genome.

Blastocyst injection is a commonly used technique to generate transgenic animals. Embryonic Stem (ES) cells reintroduced into host blastocysts can contribute to all adult tissues, including germ cells. After blastocyst injection, embryos are reimplanted in a surrogate mother. The animal obtained from injected blastocysts is made of cells of two origins (host blastocyst derived cells and injected Embryonic Stem cells) and is called a chimera. Therefore, a genetic modification introduced in Embryonic Stem cells by homologous recombination can be introduced in the germ line of a chimera (such as a chimeric rodent, for example a chimeric mouse) and be transmitted to progeny.

The chimeric rodent may be crossed with a wildtype rodent. The offspring (the F1 generation) may be screened for the presence of one or more of the transgenes.

The F1 generation, such as the F1 generation of the APP/tau double knock-in rodent, or any offspring thereof, may be used to generate single knock-in transgenic rodents by excising specifically one of the transgenes. This can be achieved by introducing a recombinase specific for the excision sequences flanking the relevant transgene. For example, the F1 rodents may be crossed with rodents expressing an excision enzyme which specifically recognizes one of the sets of excision sequences as describe above (for example the Cre recombinase or the Flippase recombinase), leading to the targeted excision of one of the transgenes. Offspring may be obtained. The offspring may be tested to confirm the excision of the relevant transgene.

The chimera may also be used to generate single knock-in transgenic rodents by excising specifically one of the transgenes. This can be achieved by introducing a recombinase specific for the excision sequences flanking the relevant transgene. For example, the chimera may be crossed with rodents expressing an excision enzyme which specifically recognizes one of the sets of excision sequences as describe above (for example the Cre recombinase or the Flippase recombinase), leading to the targeted excision of one of the transgenes. Offspring may be obtained. The offspring may be tested to confirm the excision of the relevant transgene.

The APP/Tau double knock-in transgenic rodent described herein may be used to generate triple knock-in transgenic rodents. This can be achieved by crossing the APP/Tau rodent with a rodent carrying a further transgene, for example a rodent carrying a presenilin transgene or a mutated form thereof. An example is a PSEN line, such as PS1-A246E transgenic mice, generated from a APP/PSEN line, which is commercially available from The Jackson Laboratories. Other suitable rodent lines, and in particular mouse lines, are known in the art. Offspring (F2 generation) may be obtained. The (F2 generation) offspring may be tested for the presence of one or more of the three transgenes.

One may also use the chimera of the APP/Tau double knock-in rodents to generate triple knock-in transgenic rodents. The resulting offspring may then screened for animals expressing all three transgenes.

The triple knock-in rodents, such as for example APP/tau/PSNE1 mice, may then be used to generate other double knock in lines by excising one of the transgenes as described above. One can thus generate lines such as for example APP/PSEN1 doubles or tau/PSEN1 doubles.

The products and methods described herein thus provide a flexible and straight forward system for the generation of various single, double or triple knock-in rodents by adding transgenes to and/or excising transgenes from the APP/tau double transgenic rodent utilising the excision sequences flanking the transgenes (One may even remove both transgenes.)

The presence or absence of transgenes may be tested by methods known in the art, such as for example PCR. The presence of the transgenes may also be tested via the encoded and expressed protein using immunocytochemistry, employing for example antibodies specific for either APP, tau, presenilin or other relevant proteins.

Transgenic rodents obtained or obtainable by the methods described herein are also embraced by the current invention.

The transgenic rodents described herein, such as for example the PBL1 triple mouse, offer a wide range of advantages and are likely to answer many key questions in AD research. The more subtle phenotype observed makes it particularly relevant to studies on early biomarkers, the development of sensitive translational procedure and related studies on early treatment strategies.

The transgenic rodents described herein may thus be used as AD animal models. There is provided a transgenic rodent as described herein for use as an AD animal model.

### Methods of Modelling

As the transgenic rodents described herein display a variety of Alzheimer's diseases related phenotypes, the transgenic rodents of the present invention are useful as Alzheimer's disease animal models.

In a further aspect there is provided a method of modelling Alzheimer's disease by providing the transgenic rodent described herein and monitoring changes in one or more of the phenotypes of the rodent.

In a further aspect there is provided a method of modelling one or more phentypes or pathologies associated with Alzheimer's disease by providing the transgenic rodent described herein and monitoring changes in one or more of the phenotypes or pathologies of the rodent.

### Methods of Screening

The transgenic rodents described herein may be used in methods of screening or assessing current or potential pro-cognitive drugs e.g. by use of otherwise conventional psychopharmacological or neuroanatomical methods.

The methods can serve either as primary screens, in order to identify new inhibitors/modulators of the Alzheimer's disease, or as secondary screens in order to study known inhibitors/modulators in further detail.

Using the transgenic model systems, a compound suspected of having a therapeutic effect in relation to Alzheimer's disease, can be administered to the animal, and any effects on the condition (e.g. change in relevant phenotypes such as behaviour, physiology, neuroanatomy, etc, and especially improvements in behavioural symptoms, (or any other suitable indicator) can be studied. The rodents are thus useful in testing the efficacy of such compounds in a pharmacokinetic context.

Preferably, the compound is tested with respect to two or more effects, e.g. behaviour plus neuroanatomy or physiology using PET.

For neuroanatomy, generally speaking, a drug to be tested is administered to a control animal or group of animals which are not the transgenic animals of the invention and simultaneously to transgenic animals of the invention. The drug may be continuously administered over a period of time. After administering the drug for a sufficient period of time the control animal(s) along with the transgenic animal(s) are sacrificed. Examination of the brain of the animals is made as known in the art.

Transgenic non-human mammals of the invention may thus be used for experimental purposes in studying Alzheimer's-like diseases, and in the development of therapies designed to alleviate the symptoms or progression of such conditions. By “experimental” it is meant permissible for use in animal experimentation or testing purposes under prevailing legislation applicable to the research facility where such experimentation occurs.

The mammals are thus useful in testing the efficacy of such drugs, in a pharmacokinetic context.

Generally speaking, a drug to be tested is administered to a control animal or group of animals which are either not the transgenic animals of the invention (wild-type) or to transgenic animals of the invention in which one or more of mutated disease associated genes have been excised, in each case simultaneously with transgenic animals of the invention in which mutated disease associated genes are not excised. The drug is preferably continuously administered over a period of time which is normally sufficient to effect the formation of aggregates in the brain of the animal. After administering the drug for a sufficient period of time the control animal(s) along with the transgenic animal(s) are sacrificed. Examination of the brain of the animals is made as described above. Comparative drug testing protocols known to those skilled in the art can be used in connection with the transgenic mammals of the invention in order to test drugs. The final intracellular concentration of the drug may be selected to be appropriate to the precise disease protein and drug in question, but may be in a range which will ultimately be appropriate for clinical usage in terms of toxicity, uptake etc. (e.g. 1 μM-1 mM more preferably 4-600 μM).

### Therapeutics and Modes of Administration

Performance of a screening assay method according to the various aspects above may be followed by isolation and/or manufacture and/or use of a compound, substance or molecule which tests positive for ability to interfere with or modulate disease related protein aggregation.

The compounds thus identified may be formulated into compositions for use in the diagnosis, prognosis or therapeutic treatment of Alzheimer's disease or the like. Thus, the present invention also extends, in further aspects, to pharmaceutical formulations comprising one'or more inhibitory or modulatory compound as obtainable by a screening method as provided herein.

Following the identification of a substance or agent which modulates or affects such protein aggregation, the substance or agent may be investigated further.

A compound which has been identified as described above, may be manufactured and/or may be used in the preparation, i.e. the manufacture or formulation, of a composition such as a medicament, pharmaceutical composition or drug. These may be administered to individuals.

Generally, an inhibitor or modulator according to the present invention is provided in an isolated and/or purified form, i.e. substantially pure. This may include being in a composition where it represents at least about 90% active ingredient, more preferably at least about 95%, more preferably at least about 98%. Such a composition may, however, include inert carrier materials or other pharmaceutically and physiologically-acceptable excipients. As noted below, a composition according to the present invention may include in addition to an inhibitory/modulatory compound as disclosed, one or more other molecules of therapeutic use.

The present invention thus extends, in various aspects, to a pharmaceutical composition, medicament, drug or other composition comprising a substance of the invention as described above, a method comprising administration of such a composition to a patient, e.g. for treatment or prophylaxis of Alzheimer's disease or an Alzheimer's disease-like condition, use of such a substance in the manufacture of a composition for administration, e.g. for treating Alzheimer's disease or similar treatment, and a method of making a pharmaceutical composition comprising admixing such a substance with a pharmaceutically acceptable excipient etc. as discussed below.

Examples of techniques and protocols mentioned above can be found in Remington's Pharmaceutical Sciences, 16^{th} edition, Osol, A. (ed), 1980.

Whether it is a polypeptide, antibody, peptide, nucleic acid molecule, small molecule, mimetic or other pharmaceutically-useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES AND TABLES

Table 1: Summary of genotype effects and interactions (genotype×frequency band) at 5, 9 and 13 months of Age during wake, REM and NREM (light phase), for prefrontal cortex (PFx) and right hippocampus (RH). All freq: all frequencies.

Table 2: Summary of age effects and interactions (age×frequency band) at 5, 9 and 13 months of age during wake, REM and NREM (light phase), for prefrontal cortex (PFx) and right hippocampus (RH). All freq: all frequencies.

FIG. 1: Generation of PLB1 Triple mice via knock-in of a APP-Tau construct followed by crossing with a homozygous PSEN line. For further information, see text.

FIG. 2. mRNA expression of human APP and Tau transgenes are stable over time in PLB1. The mRNA expression of either human transgene (A: hAPP; B: hTau) was not significantly different in hemi- or homozygote animals at either timepoint, whereas heterozygotes showed 2-3 fold lower expression levels. General expression levels of human APP are about 3 fold higher than human Tau expression. Shown are mRNA copy numbers normalised to mouse GAPDH expression as endogenous control (geometric mean±SEM). Significances indicated are from unpaired t-tests (** p<0.01.

FIG. 3. Mutant amyloid precursor protein expression in PLB1 mice. Coronal sections from heterozygous (Het) and himizygous (Hemi) PLB1 Triple animals at 6, 10, 12 and 14 month of age, labelled with DE2B4 antibody targeted toward the 13 amyloid 1-17 peptide sequence, see methods for full details. Staining was seen in soma and processes in hippocampal pyramidal cells (CA1) and cortical neurons (Crtx). Occasional strong extracellular immunostaining demonstrats the formation of amyloid plaques (insets). Cortical plaques were also visualised by fluorescence reactivity of Congo Red (top) and Thioflavin-S(bottom), hemi only. Positive reactivity demonstrates the presence of β-sheet protein folding and mature aggregation of extracellular β amyloid deposits. The presence of such plaques was sparse (<6 per section). Both intracellular and extracellular immunoreactivty was seen across all ages tested, with greater staining present in hemizygous brains compared to heterozygous brains reflecting increased gene expression. Images taken at 40× magnification, 50 μm illustrated by white bar.

FIG. 4. Mutant Tau expression in PLB1 mice. Coronal sections labelled with HT-7 antibody targeted toward the human specific sequence, see methods for full details. Staining was seen in somas and processes in hippocampal pyramidal cells (CA1) and cortical neurons (Crtx). A progressive loss of organised neurtic staining is seen with age and an increased Ht-7 cortical neuron count is also seen between hets and hemi/homos. Wild type littermate sections are shown as negative controls, note minimal cross reactivity of antibody with endogenous tau. Images taken at 40× magnification, 50 μm illustrated by white bar.

FIG. 5. Phospho-tau immunoreactivity in PLB1 mice. Immunoreactivty of coronal sections labelled with antibodies targeted towards selectively phosphorylated residues of tau. A) PS396 strong immunoreactivity was present within in soma of CA1 hippocampal neurons and cortical neurons. Weaker staining is also present in neurites, particular evident in the CA1 of younger mice. With age, neuritic staining becomes disorganized and fragmented (arrows), before disappearing. B) AT-8 positive staining is initially seen in cortical neuronal somas (Crtx) and increases with age. CA1 somatic staining is detected from 12 month of age, with subtle neuritic staining in both cortical and hippocampal regions. Images taken at 40× magnification, scale bar: 50 μm.

FIG. 6: Electrophysiological characterisation of PLB1 triple transgenic mice. A-D) Input-output (10) curves of basic synaptic transmission in the CA1 region of hippocampal slices from Triple transgenic mice at 6 and 12 months of age compared to wild-type (WT). The fEPSP slope was plotted against stimulation intensity and presynaptic fibre volley amplitude. E-F) Paired pulse responses of fEPSPs (ISIs: 10, 40, 100 and 200 msec), expressed as the second fEPSP slopce (S2) calculated relative to the first (S1). PPI was intact in transgenic slices at both age groups, while PPF was significantly reduced (P<0.05 for msec; P<0.01 for ISI of 200 msec at 6 months; P<0.05 for ISI of 40 msec and P<0.01 for ISI of 100 msec at 12 months). G-H) LTP (fEPSP as % of baseline) from Triple mice at 6 and 12 months compared to WT. LTP was significantly impaired at both age groups (P<0.0001, ### interaction at 6 and 12 months). Sample fEPSPs are included in all figures, the arrow indicates tetanisation.

FIG. 7. A) Distance moved during first 3 hrs of habituation in the PhenoTyper cages (in 10 min bins). PLB1 wild types (WT) habituated faster at 9 months compared to Triples. B) Average distance moved during 5 days (excluding 2 days habituation) in the PhenoTyper cages in 1-hr bins for a 24 hour light-dark cycle. A significant genotype effect was noticed at the age 5 month. Int: Interaction. For mean data, see FIG. 19.

FIG. 8. Sleep architecture in WT and PLB1-triples in a longitudinal study. A comparison of time (in %) spent awake (A), in REM (C), and NREM (D) revealed a significant overall genotype effect. Paired comparison indicated this be also significant during wake, REM and NREM in 13 month PLB1-triples. Onset of sleep) sleep latency, B) was also delayed in the Age group. The relative occurrence and distribution histograms of NREM events in Triples (E) and WT (F) suggest an age-dependent increase of short NREM events in triples.

FIG. 9: EEG Power spectra in PLB1 mice: Genotype Effects. Selective genotype effects observed in different frequency bands (see Table 1 for summary) during the light phase, in the prefrontal cortex (PFx) and right hippocampus (RH). At 5 month, a significant genotype effect was detected during PFx wake (A) REM (B), NREM (C) and RH REM (D) spectra. At 9 and 13 month age-group selective a genotype effects were represented for different vigilance stages of PFx and RH (E-L).

FIG. 10: EEG Power spectra in PLB1 mice: Age Effects. Selective Age-effect observed in different frequency bands. In PFx wake, delta and beta range of frequencies depicted an age-effect in the triples (B) whereas WTs (A) do not show significant alterations. During wake and REM in RH, an age-effect was noticed in the WT (C,E) whereas triples (D,F) do not show any age-related significant alterations.

FIG. 11: Object recognition in PLB1 mice. A: Arena set up and exploration pattern (movement trajectory) during habituation with a central object X (left), and during object recognition, depicting exploration of a familiar object Y and a novel object Z (right). B: Time (in %) spent with the novel object in PLB1 wild type (WT) and Triple mice at 8 and 12 months of age. Significances are indicated between groups (*: p<0.05; **:p<0.01), and for comparison with chance level ($: p<0.05; $$: p<0.01; $$$: p<0.001).

FIG. 12: Social recognition behaviour in PLB1 mice at 5, 9 and 13 months of age. A: Activity measured during the habituation phase (mean distance moved) revealed an age related reduction in both wild type (WT) and Triple animals, but no genotype differences. B: During the sociability phase, all groups spent significantly more time in the immediate vicinity of an unfamiliar mouse (S) compared to the empty compartment (E). C: During the social memory phase, WT discriminated between the familiar first stranger (S1) and a novel stranger (S2) in all age groups. PLB1 Triples only discriminated between the conspecifics at 5 months of age. Data are shown as time spent (mean, in seconds) in the immediate vicinity. ns: not significant. *: p<0.05; **: p<0.01, ***: p<0.001.

FIG. 13: FDG-PET images of PLB1 TRIPLE vs. WT animals at 5 (top) and 15 (bottom) months of age. Left, rear and right views through a 3D rendered object showing areas of significant increase (red) and decrease (blue). A surface render of a typical CT image from one of the animals is also shown to provide an anatomical reference.

FIG. 14: A: Genotyping PCR of the F1 generation. The genotypes of the 36 pups derived from the F1 breeding were tested by PCR using the primer combinations detecting the targeted Hprt allele. 5 of the 36 tested animals were identified as being heterozygous for the Hprt knock-in. PCR using DNA from the targeted ES clone #5B10 was used as positive control. PCR without template served as a negative control. M: 1 kb DNA-Ladder (NEB) B: Southern blot analysis of the F1 generation. The genomic DNA of the 2 tested F1 mice (#17763, #17764) were compared with wild-type DNA (129ES, BL6). The NheI digested DNAs were blotted on nylon membrane and hybridised with either the 5′ probe (A) to validate the zygocity of the Hprt gene mutation in these animals.

FIG. 15: Southern blot analysis of the F1 generation. The genomic DNA of the 2 tested F1 mice (#17763, #17764) were compared with wild-type DNA (129ES, BL6). The NheI digested DNAs were blotted on nylon membrane and hybridised with either the 5′ probe (A) to validate the zygocity of the Hprt gene mutation in these animals.

FIG. 16: Weight of PLB1 animals (males and females) and WT littermates. In addition to an overall effect of gender, a significant age effect was noticed in both male and females. Data expressed as mean±SEM.

FIG. 17: Motor performance in PLB1 mice. A: Rotarod: Mean time of active performance sustained on the rotating rod for each genotype and age group across eight training trials on two consecutive days. SEM omitted for clarity. There was no difference between the tested cohorts. B: Balance Beam: Latency to reach the end of a 50 cm long beam of different size (5-28 mm) and shape (square or round). There was no difference between genotypes. Data are expressed as mean value +/−SEM.

FIG. 18: Wild type sections and sections from a APP/PSEN overexpressing mouse (JAX, 12 m) are shown as negative and positive controls for APP antibody DE2B4 and tau antibody H7. Bar. 50 μm.

FIG. 19: Locomotor activity (mean distance moved) in PLB1 mice. A: During 3 hrs of habituation and 5 days in the Phenotyper (B), averaged for light and dark phase (12 hrs each) for PLB1 Triples and wild type (WT) at 5, 9 and 13 months of Age.

FIG. 20. Normalized power spectra (0-50 Hz) of Wake, REM, and NREM stages observed in the PFx and right hippocampus during the light phase.

FIG. 21: Example horizontal, parasagittal and coronal slices through a CT image from one of the PLB1 animals. Areas of significant increase (red) and decrease (blue) in metabolism of the aged Wild Type group relative to the young Wild Type group are shown superimposed on the CT image. The blue lines show the location of the slices in all three planes.

FIG. 22: Example horizontal, parasagittal and coronal slices through a CT image from one of the PLB1 animals. Areas of significant decrease (blue) in metabolism of the aged Triple group relative to the young Triple group are shown superimposed on the CT image. The blue lines show the location of the slices in all three planes.

FIG. 23: Example horizontal, parasagittal and coronal slices through a CT image from one of the PLB1 animals. Areas of significant decrease (blue) in metabolism of the young Triple group relative to the young Wild Type group are shown superimposed on the CT image. The blue lines show the location of the slices in all three planes.

FIG. 24: Example horizontal, parasagittal and coronal slices through a CT image from one of the PLB1 animals. Areas of significant decrease (blue) in metabolism of the old Triple group relative to the old WT group are shown superimposed on the CT image. The blue lines show the location of the slices in all three planes.

FIG. 25: Information on APP, amyloid and Tau

FIG. 26: Sequence similarity between human and mouse 97% (Shin&Ji 2007)

FIG. 27: Isoform composition of normal adult brain Tau

FIG. 28: Double mutated APP derived from isoform a (APP770)

FIG. 29: Tau coding sequence with P301L and R406W mutations (derived from NM_{—}016835 sequence)

### EXAMPLES

### Material and Methods

Information on APP, amyloid and Tau

Human inserted transgene APP
APP mRNA sequence used: NM_{—}000484
Protein 770 amino acids
Full length protein glycosylated approx 120 kDa
Amyloid
   monomer approx 4 kDa (36 to 42 amino acids—seen on high % PAGE)
   dimer 8 kDa
   trimer 12 kDa (reference for mono to trimer: McLean et al 1999, Ann Neurol)
   oligomer (56 kDa Lesne 2006)
      Source abcam

http://www.abcam.com/Amvloid-beta-precursor-protein-antibodv-ab12269.html

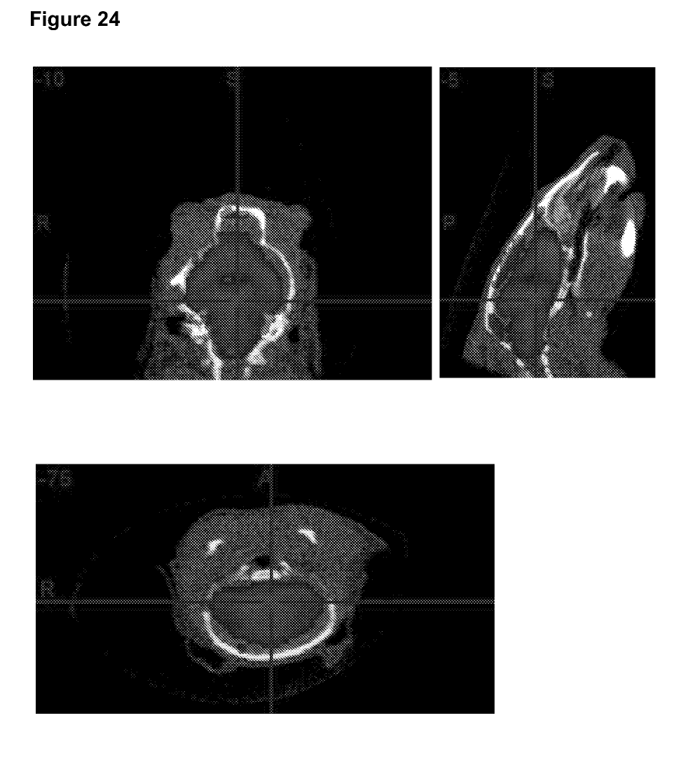

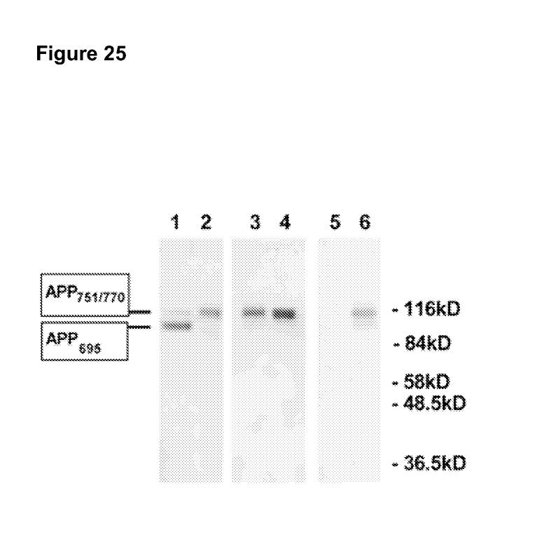

### [See FIG. 25]

### Other Human Forms:

### APP:

751 AA—130 kDa (glycosylated) 110 immature

### 695 AA—90 kDa

Amyloid same as above as these isoforms only differ in NTD (Kunitz and Ox sequences)

### Sequence Similarity Between Human and Mouse 97% (Shin&Ji 2007)

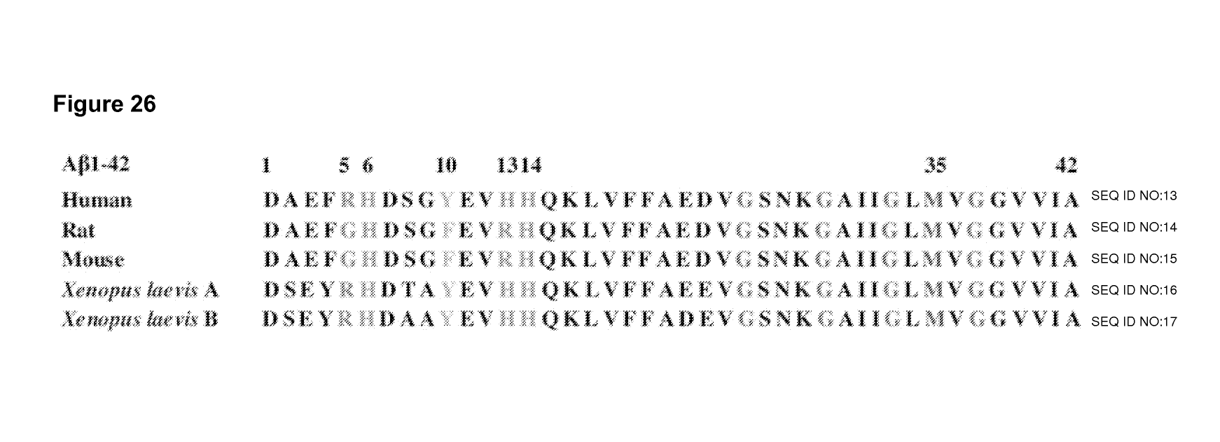

### [See FIG. 26]

Size of amyloid varies at CTD—1-42 supposedly most toxic one 1-40 more abundant Ratio of the two shifted towards more 1-42 in AD

### Human Inserted Transgene Tau

Tau mRNA sequence used NM_{—}016835.3
Protein 441 amino acids
Full length protein approx 60 kDa (Rametti et al. 2004)

### Tau

6 isoforms from Hong et al 1998

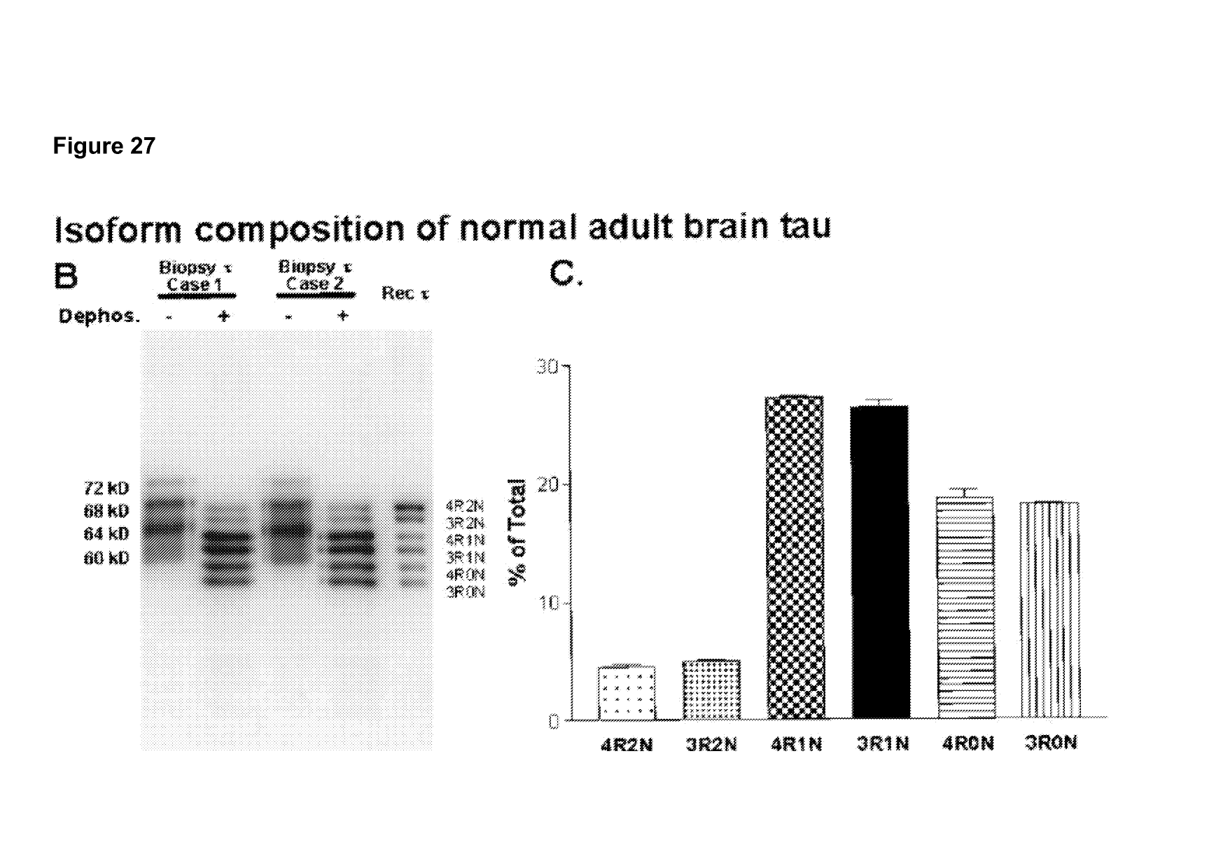

### [See FIG. 27]

Goedert&Spillantini 2000 review tau isoforms give 6 distinct band—sizes on gel between 50 and 72 kDa

### Cross Species

McMillan et al 2008, J Comp Neurol—mouse equivalents run faster on PAGE than human ones

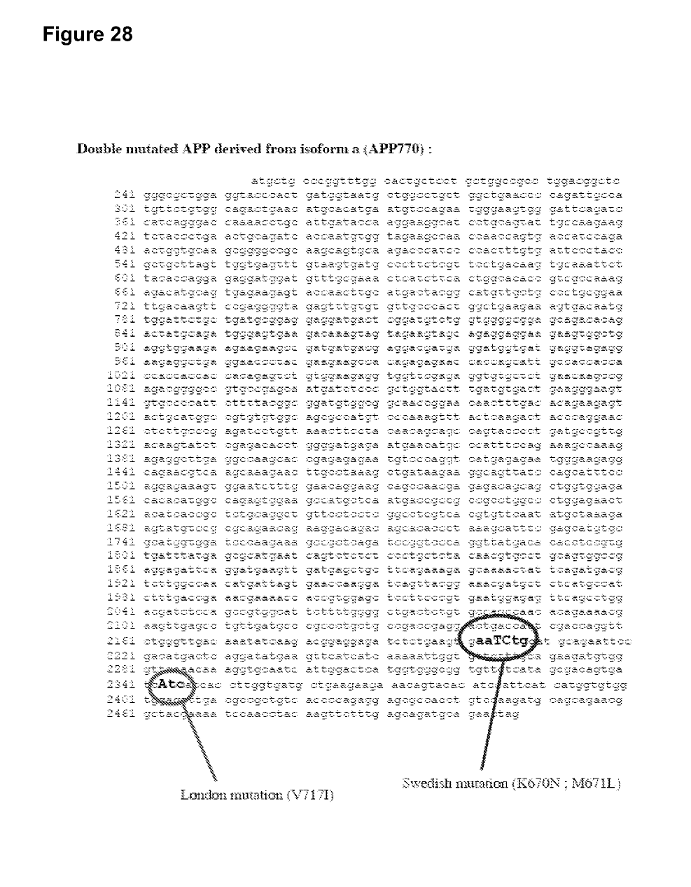

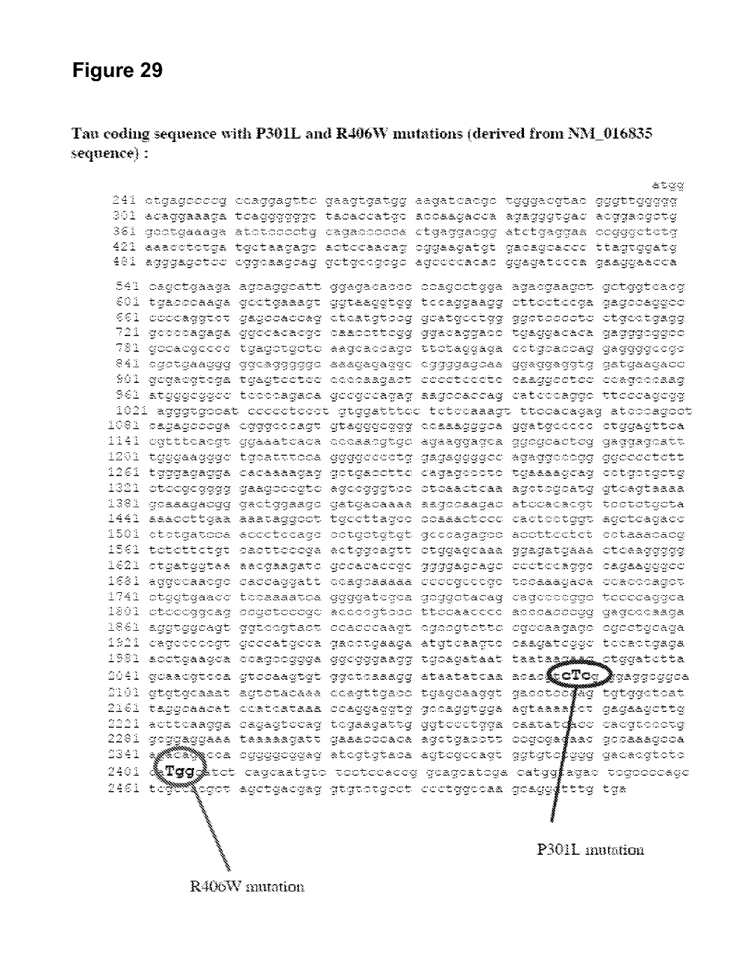

### [See FIGS. 28 & 29]

### 1. Animals

All animal handling was performed under the University's Code of Practice on the Use of Animals in Research as well as the legal requirements of the Animals Act 1986 and Home Office Code of Practice guidance. Mice were kept on a 12:12-h light-dark cycle, and experiments were conducted during the light phase of the cycle, unless stated otherwise.

### 1.1. Generation of PLB1 Mice

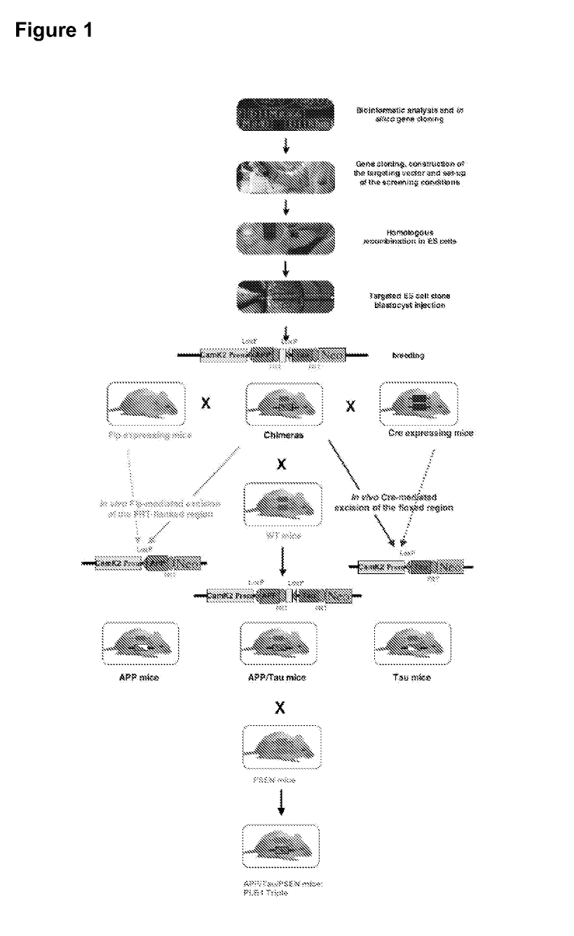

The human APP (isoform 770, NM_{—}000484) with a triple mutation [Swedish (K670N; M671L) and London (V717I)] and human four-repeat tau (NM_{—}016835) with P301L and R406W mutations cDNA, driven by the mouse CaMKII promoter (CaMK2) was cloned into the HPRT™ targeting vector (genOway). An artificial intron derived from a pNN265 vector was fused to the CaMKIIα promoter, to stabilise the expression of the transgenes. An internal ribosome entry sites (IRES) nucleotide sequence was also introduced between the APP and Tau transgenes, to allow translation initiation and protein synthesis of APP and tau proteins from a single mRNA strand. Additionally, the transgene also contained LoxP and FRT sequences, flanking APP and Tau cDNAs, respectively. The LoxP and Frt sequences allow the selective deletion of either the tau cDNA or the APP cDNA and to generate single tg models from the same targeted embryonic stem cell (10× and frt, see FIG. 1).

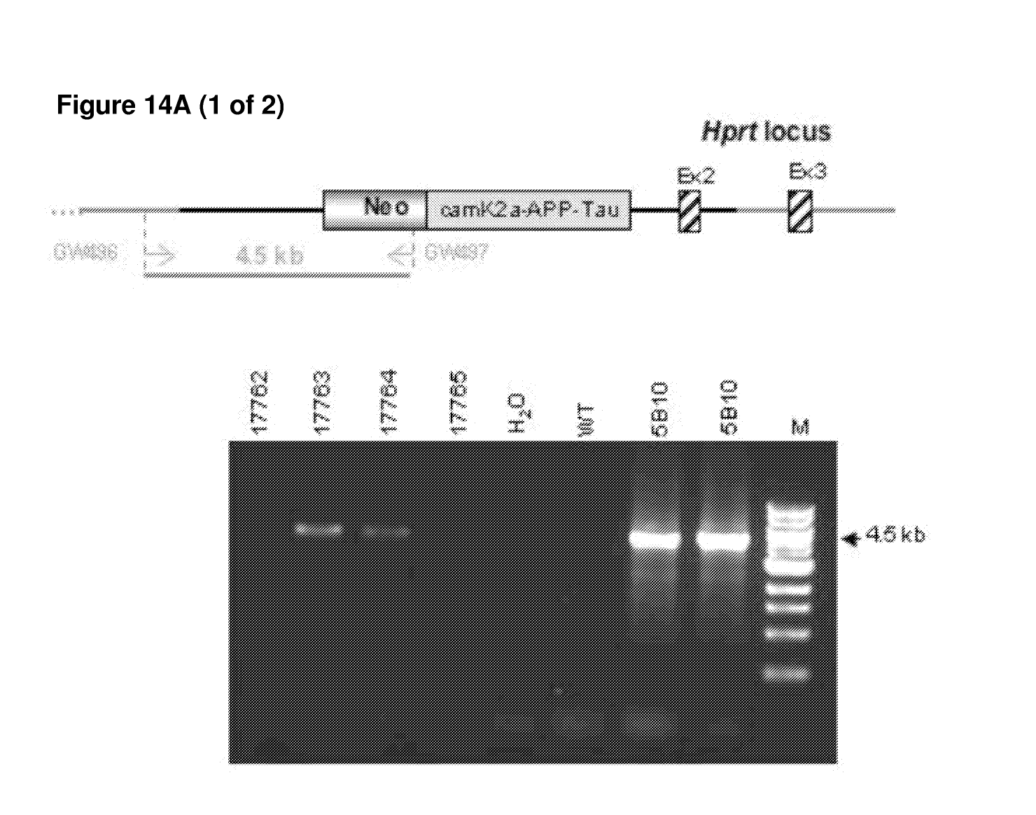

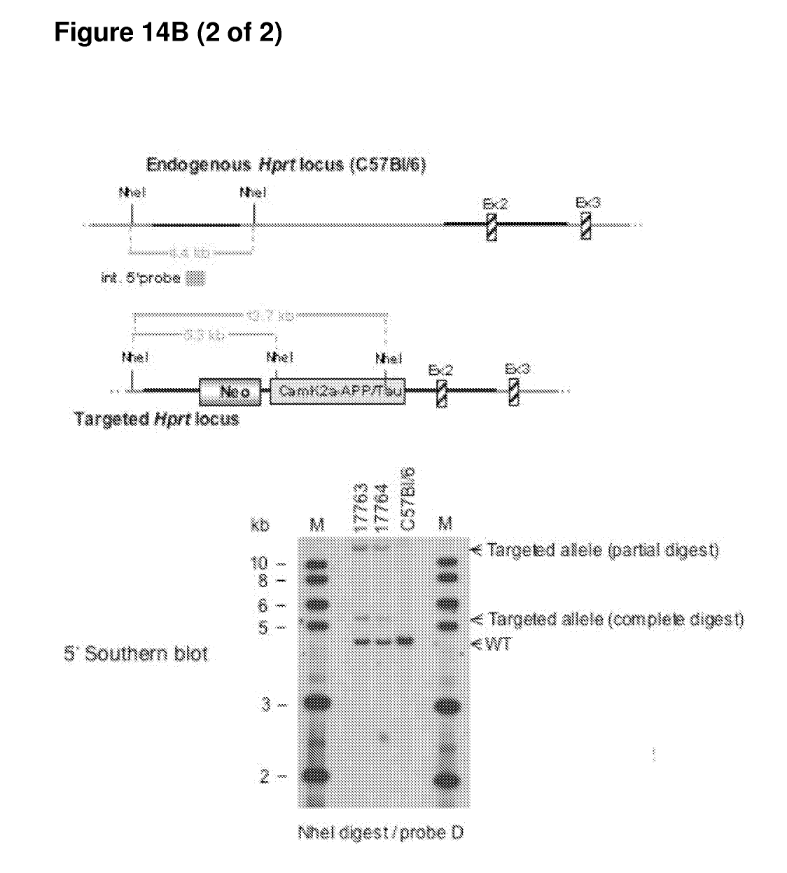

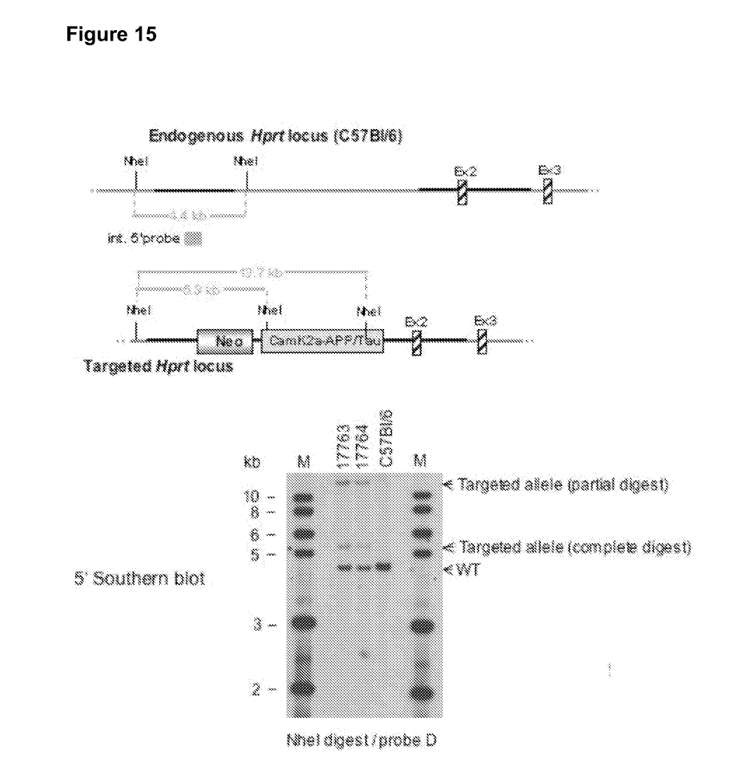

Gene targeting was performed in E14Tg2a ES cells derived from 129P2/Ola mice. After the injection of E14Tg2a ES cells into C57BL6/J blastocysts, chimeras of two different cell types were obtained. Genotyping with PCR was conducted that allowed the detection of the junction between the HPRT locus and the 5′ homologous recombination of the targeting vector, for the identification of the targeted allele within the F1 generation. Validation of transgenic status of F1 and F2 generations was achieved using PCR and Southern Blot analysis (see FIGS. 14 and 15).

F1 animals were crossed with a PSEN line (PS1-A246E transgenic mice, generated from a APP/PSEN line purchased from The Jackson Laboratories (Jax), Borchelt et al., 1999—see below for a more detailed discussion) to generate PLB1 Triple animals (PSEN/APP/Tau). The Jax APP/PSEN line (RD mutation carriers removed) also served as a positive control for amyloid histology.

Genotyping for the detection of the targeted Hprt allele was achieved by PCR amplification over the 5′ short arm of homology using a forward primer GW496 (5′-ACA ATT GCC TGT GAA TCA AGT TCT AGA TCT GG-3′) hybridizing upstream of the targeting vector homology sequence and a reverse primer GW497 (5′-TTC GTC CAG ATC ATC CTG ATC GAC AAG AC-3′) hybridizing within the neomycin selection cassette (see FIG. 14). Because of its localisation, this primer pair allows the specific detection of the 5′ integration of the targeting vector within the Hprt locus. This procedure allowed the establishment of a genotyping PCR sensitive enough to detect 1 genomic copy within the genomic DNA. Additionally, genotyping with Hprt WT primer pairs was also conducted (5′-TGT CCT TAG AAAACA CAT ATC CAG GGT TTA GG-3′ and 5′-CTG GCT TAA AGA CAA CAT CTG GGA GAA AAA-3′), to distinguish between hetero- and homozygous animals. Genotyping for PSEN was conducted as per standard instructions provided by The Jackson Laboratories.

### Generation of PSEN1_{(A246E)} Homozygous Mice

A breeding population of homozygous PSEN1_{(A246E)} mice has been established from transgenic mice expressing APPS_{swe} and PS1_{(A246E)} transgenes (independent mutations, Borchelt et al., 1997), purchased from The Jackson Laboratories (B6C3-Tg(APPswe, PSEN1dE9)85 Dbo/J). This strain was bread with C57BL6 to generate a homozygous PSEN1 line, and to remove a C3-related retinal dystrophy mutation. Mice that are heterozygous for both APP_{swe} and PSEN1_{(A246E)} transgenes reveal the histological presence of β-amyloid (βA) deposits at around 9 months of age. APP_{swe} (no PSEN1) develop some plaques around 18 months (see also: Borchelt et al., 1997). The PSEN1 line does not develop plaques, appears normal in behavioural experiments (water maze, object recognition, general activity (Phenotyper) and social interaction) and hippocampal physiology (basic synaptic transmission, long-term potentiation, paired-pulse responses and sensitivity to carbachol) up to 12 month of age (age groups test: 3, 6 and 12 month) (Borchelt D R et al. Neuron. 1997 October; 19(4):939-45).

### 2. RNA Extraction and Quantitative Real-Time PCR

Forebrain samples (4 mm³) were dissected from 6 months and 12 months old PLB1 mice [6 months: n=5 each; 12 months: n=4 (heterozygous, hemizygous and WT) or n=3 (homozygous), and immediately stored in RNA Later solution (Qiagen Sussex, UK) at 4° C. overnight, then at −20° C. until extraction. Total RNA was extracted with RNeasy Lipid Tissue Mini Kit (Qiagen, Sussex, UK) according to manufacturer's instructions, including homogenisation via Qiashredder and on-column DNasel (Qiagen, Sussex, UK) digests. Only integrity-controlled RNA (Agilent 2100 Bioanalyzer, Agilent Technologies UK Ltd., Cheshire, UK, RIN-score >7) was used for cDNA synthesis (from 2 μg total RNA) with the Transcriptor High Fidelity Reverse transcriptase kit (Roche, Burgess Hill, UK). Gene expression analysis was carried out with the Bio Rad MiniOpticon Real-Time PCR Detection System using iQ SYBR Green Supermix (BioRad, Hemel Hempstead, UK) in a final volume of 20 μl. 100 ng cDNA equivalent were run in triplicates per sample with 3.2 μM each gene specific oligomer primers: human APP: 5′-ACT GGC TGA AGA AAG TGA CAA-3′ (forward) and 5′-ATC ACC ATC CTC ATC GTC CTC G-3′ (reverse); human Tau: 5′-CAC GGA CGC TGG CCT GAA AG-3′ (forward) and 5′-CTG TGG TTC CTT CTG GGA TC-3′ (reverse) and as housekeeping gene mouse GAPDH: 5′-ACT TTG TCA AGC TCA TTT CC-3′ (forward) and 5′-TGC AGC GAA CTT TAT TGA TC-3′ (reverse). The thermocycler profile consisted of 3 min at 95° C. initial denaturation followed by 36 cycles of 30 s at 95° C., 20 s at annealing temperature (APP 65° C., Tau 63° C., GAPDH 60° C.) when the fluorescence was monitored. As controls a negative cDNA reaction (excluding reverse transcriptase) was run from each sample and also a no template control was included for each PCR run. Quantification was obtained by comparing the fluorescence intensities against standard serial dilutions of plasmids containing 20 to 2×10⁵ copies of the transgenes, or 2×10³ to 2×10⁷ copies of the housekeeping gene. Melting curve analyses ensured specific amplification products of the samples. Data analyses were performed using the Opticon Monitor™ Software (Bio Rad, Hemel Hempstead, UK). Absolute gene expression (copy number) of triplicate means were normalised to the mean of the endogenous controls. Experimental data are expressed as geometric means±S.E.M. Statistical analyses were performed with Prism software (V.5, GraphPad Software Inc., San Diego, USA) using an overall ANOVA followed by unpaired t-tests for comparison of selected data pairs. P′ s<0.05 were considered significant.

### 3. Tissues Harvesting and Histology

Mice (n=3 (PLB1 triple) and n=2 (WT) per age group and genotype) were terminally anaesthetised and intra-cardially perfused with 4% paraformaldehyde in 0.1M phosphate buffer. Brains were removed, post-fixed overnight, wax embedded and sectioned. Slide mounted 5 μM thick coronal sections were used for either DAB based immunochemical staining or for fluorescence based 13-sheet protein staining. Diaminobenzidine (DAB) based immunocytochemical staining was conducted with a Leica/Bond autostainer (Leica Microsystems, Milton Keynes, UK). Sections underwent automated dewaxing, acidic antigen retrieval and appropriate antibody application.

Immuno-labelling for amyloid was conducted using DE2B4 (1:200, Abcam, Cambridge, UK). For human-specific tau, HT-7 was used (dilution 1:200, Autogen-Bioclear, Wiltshire, UK), for phospho-tau, AT-8 (targets tau phosphorylated on Ser202/Thr205, dilution: 1:25, Autogen-Bioclear), a marker for phospho-tau closely associated with filamentous tau formation (Braak et al., 1994, Noda-Saita et al., 2004; Deters et al., 2008), and an antibody targeting tau phosphorylated at S396 (PS396, 1:200, Autogen-Bioclear) were used.

Primary antibodies were visualised using Bonds refined DAB staining kit (Leica Mircosystems), nuclei were counterstained using haematoxylin.

For Congo Red fluorescence detection of (3-sheet protein aggregation, slides were dewaxed in a NaCl-saturated 80% ethanol solution for 20 mins prior to staining with 0.5% Congo Red (Sigma-Aldrich, Gillingham, UK) in NaCl-saturated 80% ethanol solution for 1 hr. Slides were dried and counter-stained with Prolong Gold with DAPI (Invitrogen, Paisley, UK) for visualisations of nuclei. For Thioflavin-S, slides were dewaxed and placed in 1% Thioflavin-S (Sigma-Aldrich) aqueous solution for 8 mins, after which slides were dried and mounted in DPX.

All images were taken with a digital camera (Axocam, Carl Zeiss; Hertfordshire, UK) mounted on a Zeiss microscope (Axioskop 2 Plus) with a water immersion lens (40×), using Axiovision software (Zeiss).

### 4. Hippocampal Slice Preparation and In Vitro Electrophysiological Recordings

Hippocampal slice preparations were modified from our previous reports (e.g. Dreyer et al., 2007). Briefly, animals were terminally anaesthetised via an IP injection of euthatal and decapitated. The brain was quickly removed into ice-cold sucrose aCSF (composition in mM): 249.2 sucrose, 1.5 KCl, 1.3 MgSO₄, 1.5 KH₂PO₄, 2.89 MgCl₂.6H₂O, 0.96 CaCl₂, 25 NaHCO₃ and 10 glucose (pH 7.4, continuously gassed with 95% O₂/5% CO₂) and the hippocampus dissected. Slices were prepared (400 μm) with a Mclllwain tissue chopper and stored in pre-warmed, oxygenated aCSF (composition in mM): 129.5 NaCl, 1.5 KCl, 1.3 MgSO₄, 2.5 CaCl₂, 1.5 KH₂PO₄, 25 NaHCO₃ and 10 glucose (32° C.) for at least 1 hour before experiments commenced. Individual slices were transferred via a pipette onto a mesh in a submerged recording chamber (Scientific Systems Design Inc., Mississauga, Canada), and an upper mesh consisting of vertical nylon threads carefully attached to the lower one to hold the tissue in place. Warmed (32° C.) and oxygenated aCSF was supplied to the recording chamber at a flow rate of approximately 5 ml per minute.

Field excitatory postsynaptic potentials (fEPSPs) were recorded in the CA1 region via an aCSF filled borosilicate glass electrode (3-7 MS)) positioned in the stratum radiatum, evoked by stimulation of the Schaffer collateral fibres with a monopolar stimulation electrode (World Precision Instruments, UK, 0.5 MΩ). The signal passed through the recording electrode to a CV203BU headstage pre-amplifier with a gain of 1 (Axon Instruments, CA, USA) connected to an Axoclamp 200B amplifier (Axon Instruments) which was set to operate in current clamp mode. A CED 1401 Plus (Cambridge Electronic Design Ltd., Cambridge, UK) digitised the analogue signal for passage to a PC, where data was acquired using the P-WIN software package (Leibniz Institute for Neurobiology, Magdeburg, Germany).

Input/output curves (10 curves) of basic synaptic transmission of fEPSP slopes were generated by stepwise increases of the stimulus intensity until saturation was reached. A paired-pulse paradigm investigated changes in presynaptic release mechanisms and short-term plasticity. Pairs of identical stimuli (inter-stimulus interval (ISI): 10, 40, 100 and 200 ms) were delivered, and the ratio of the fEPSP slope of the second response calculated relative to the first.

LTP experiments were run at a stimulus intensity of 40-50% of maximum. A pre-tetanus baseline response was recorded (responses recorded every 30 seconds), slices with variable responses were discarded (variability >10%). To induce LTP, a theta-burst tetanus was applied (5 Hz, 5 bursts of 4 stimuli (100 Hz), inter-burst interval of 200 msec for 1 second) and responses recorded for 1 hour post-tetanus.

### Data Analysis

Analysis was performed using GraphPad Prism software (V5; GraphPad Software, San Diego, Calif., USA). 10 curves of EPSP slope vs. stimulus intensity and fibre volley amplitude were generated and compared between groups via a two-way ANOVA (genotype×stimulus/genotype×fibre volley amplitude).

Paired pulse responses were calculated as the ratio of the second response relative to the first, with overall group analysis carried out via a two-way ANOVA (genotype×ISI) and post-hoc students t-tests employed to compare individual ISIs. LTP time courses were calculated relative to baseline values (=100%), with data illustrated as means±standard error of means (SEM). A two-way repeated measure ANOVA was applied to compare post-tetanus values between groups (treatment×time). Significance was set at P<0.05=* (significant), P<0.01=** (highly significant) and P<0.001=*** (extremely significant).

### 5. Circadian Rhythms, Vigilance States and Global Brain Activity (EEG)

PLB1 WT and Triple animals (both genders) were assessed at 5, 9 and 13 months of age for circadian activity, vigilance states and EEG profiles in a longitudinal study. For EEG recordings, young PLB Triple and WT animals received cranial implants for a longitudinal study at 4 months of age. Mice were anaesthetized with 3% isoflurane in medical grade oxygen and maintained on 1.5% isoflurane anaesthesia during surgery. Epidural Gold screw electrodes were implanted above the prefrontal cortex (2 mm anterior to Bregma/close to midline) and hippocampus (2 mm posterior to Bregma/1.5 mm lateral to midline). Reference and ground electrodes were placed at neutral locations on the parietal and occipital regions. Electrodes were soldered and assembled into 6 pin adaptor and fixed on the skull by a mixture of Durelon dental cement and glue. Post surgery, animals were injected with 0.5 ml saline (i.p.) and 0.01 μl Temgesic (s.c.). Animals were weighed daily and at each of the test sessions. At least 7 days were allowed for recovery before the start of the experiments. Mice were single-housed in standard macrolon cages (82 cm² free space) in a controlled holding environment with a 12-hour day-night cycle (lights on at 7 a.m.). Animals had free access to water and standard rodent food pellets.

Circadian activity measures were taken (PLB1 WT (n=34) and Triple (n=31) in PhenoTyper cages (30 cm×30 cm×35 cm) (Ethovision, Noldus, Wageningen, The Netherlands) that allow automated video-tracking via infra-red sensitive built-in cameras, and contain a fixed feeding station and water bottle. Ethovision software (V3.0) was used for video-tracking performed at a rate of 12.5 samples/second. Activity was assessed for seven days, with 2 days of habituation before EEG recordings commenced in a subset of animals.

EEG recordings were conducted for 24 hrs with wireless Neurologger units (NewBehavior, Zurich, Switzerland). The weight of the recorder in combination with the P10 hearing aid batteries is ˜3 g (approximately 10% of the body weight) and the physical dimensions are 24×15×5 mm. The device contains a built-in accelerometer to record movements. The sample rate was set to ˜200 samples per second.

### Data Analysis

Activity analysis was based on distance moved (in 10 min bins for 3 hours of habituation and 1 hr bins post habituation) and time spent in defined food and water areas of the Phenotyper cages using the Mnimi software package (in-house development). EEG data (PLB1 WT (n=10) and Triple (n=10)) were downloaded offline to a PC using a docking station. Data retrieved (in hexadecimal format) were transformed to .txt format using EEG_Process (Matlab 7, The MathWorks Inc., Natick, USA), and imported into the SleepSign software package (SleepSign for animals, Kissei Comtec Co. Ltd, Nagano, Japan) for vigilance staging (wake, REM and NREM sleep) and extrapolation of power spectrum values for 4 sec bins. Automated stage identification was followed by visual inspection and correction. Bins with movement artefacts were excluded from analyses. The EEG signal was bandwidth filtered (0.5-50 Hz) and submitted to power spectra analysis. The Fast-Fourier-Transform (FFT) was calculated for each epoch (4s) with a resolution of 0.77 Hz (sample rate 200 Hz, 256 points), Hamming window smoothed and averaged. The spectral bands of delta (0.5-5 Hz), theta (5-9 Hz), alpha (9-14 Hz), beta (14-20 Hz) and gamma (20-50 Hz) were calculated and expressed as relative power, normalised relative to the absolute maximum power over all frequencies (0-50 Hz). This normalization was used to standardise absolute power across the populations. Spectral EEG characteristics were analyzed within the states of NREM, REM or wakefulness (WAKE).

Vigilance stages were identified based on accelerometer activity and hippocampal power spectra parameter (delta and theta power). The overall time spent in different stages (in %) was calculated for 12-hr light and dark phases, as well as the relative occurrence (relative distribution of vigilance stages normalized per 1-hour) and the mean length of each event. Histograms of wake, REM and NREM were drawn to investigate the frequency distribution of events of different durations (number of events in 12 hours plotted versus their duration), as an indicator of fragmentation of vigilance stages.

Statistical comparisons was made by 2-way ANOVA planned paired comparison to determine age effects, genotype effects and age/genotype by time interactions, as well as age/genotype by frequency interactions; with post-hoc Bonferroni tests, using GraphPad Prim 5.0 (GraphPad Software Inc., San Diego, Calif. USA). Significance was determined at the level of p<0.05.

### 6. Learning & Memory

6.1. Object Recognition

The object recognition paradigm was modified from previous studies in AD mouse lines (Good & Hale, 2005). PLB1 WT and Triple animals (male and female) were tested at 8 (Triple: n=14; WT n=6) and 12 m of age (Triple: n=14; WT n=11). Subjects were single-housed during the experiment and had free access to food and water with the exception of the test sessions.

The apparatus was a white Perspex cylinder (50 cm diameter; 50 cm wall height) with a number of cue cards placed on the top rim of the wall. In addition, extra-maze cues, such as posters and laboratory equipment were present. The arena was placed in a corner of a quiet experimental room and the animal's movement were recorded by an overhead camera, stored on DVD, and digitized to a PC-observation system (Ethovision V3.1, Noldus, NL).

Objects were obtained from a variety of sources and were made of materials that could not be easily gnawed by the mice (e.g. metal and glass) or climbed on. They differed in shape and colour, were between 15-20 cm in height, and cleaned with 70% alcohol in distilled water before the start of each trial to eliminate any odour cue. Objects were placed in the centre of the arena or into each half during different stages of training. Behavioural testing consisted of: 1) Habituation: Two days in which each subject was allowed to explore the arena for 2 trials of 5 min each (inter-trial interval (III): 2 min). During trial 1, the arena was empty, during trial 2, a single object A was placed in its centre. The same procedure was repeated the following day. 2) Object novelty: On day 3, each mouse was presented with two identical sample objects B (approx 10 cm apart), placed in the middle of the two halves of the arena (sample phase). After 5 min exploration and an ITI of 5 min, objects were replaced by one identical object B and a novel object C (counterbalanced design for each group) and mice reintroduced into the arena again for 5 min (test phase).

### Data Analysis

Object exploration defined as time (in % of total exploration time) within a 4 cm radius of each object (target zone) was measured. Video-observed X-Y coordinates were analysed and parameters extracted included: i) overall activity (pathlength); ii) velocity; iii) object exploration time (in s). Data were analysed using factorial analysis of variance (ANOVA) with genotype and age as between subject factors and object as within-subject factors followed by Tukey's post hoc analysis. P′ s<0.05 were considered reliable.

6.2. Social Interaction & Social Memory

PLB1 WT and Triple (male and female) animals were tested at 5 (n=42 and 52, respectively), 9 (n=37 and 51, respectively) and 13 months of age (n=34 and 45, respectively) in a social recognition and memory paradigm (adapted from: Nadler et al., 2004). The social testing apparatus was a three-chambered Perspex box (chamber size: 20 cm×42 cm×22 cm) with dividing walls containing a door (8 cm in diameter) for access into each chamber. Both side-chambers contained a cylindrical cage for placement of a conspecific mouse (stranger mouse). The cage permitted visual, olfactory, auditory, and some tactile contact between the stranger and the test mouse, but prevented aggressive interactions. A target area for social interaction was defined based on the optimal distance for subject mice to sniff at a stranger inside the cage (4 cm). Subject trajectories were video-recorded using the Ethovision system (Ethovision 3.1, Noldus, Netherlands; sample rate: every 12.5 Hz). Data analysis was conducted off-line.

Each test session consisted of a habituation, sociability (one stranger, gender matched) and social memory phase (novel and familiar stranger), each lasting 10 mins (ITI: 5 mins). Strangers remained in the same geographical location for one subject to avoid potential confusion due to smell caused by swapping, but strangers' positions were randomized across subjects. After every stage of social recognition, the floor of the arena was cleaned with 70% ethanol.

### Data Analysis

During all phases activity was monitored (as distance moved). To score sociability, data were analysed as time spent in the vicinity zone of stranger 1 (S1) relative to the corresponding empty compartment. For social memory, time spent with the novel stranger (S2) relative to S1 was calculated, as well as a discrimination ratio.

Statistical analysis was performed using Graph Pad Prism (V5.01). Repeated measures 2-way ANOVA was used to determine overall levels of significance. As post test, paired (within group) and unpaired (between groups) t-tests were used. P′ s<0.05 were considered significant.

### 7. PET Imaging

Four groups of mice (all male) were used in the PET/CT acquisitions: Young WT mice (n=10), young PLB1 Triple mice (n=11), aged PLB1 WT mice (n=7) and aged Male PLB1 Triple mice (n-12).

The i.p. ¹⁸F-FDG administration was performed in conscious, fasted animals, uptake occurred in the dark over 45 mins. The animals had access to drinking water at all times. Mice were kept warm by placing the cage on a heating pad kept at 35° C. and warming started at least 30 minutes before ¹⁸F-FDG injection and continued during the ¹⁸F-FDG uptake period. In the pre-imaging period, ¹⁸F-FDG (range: 9.84-17.32 MBq) was intraperitoneally injected (injected volume 0.33-0.5 ml).

For PET/CT imaging, animals were anesthetized with ketamine 100 mg/mL (Vetalar* V®)/medetomidine 1 mg/mL (Domitor®)/sterile water solution, and placed on the bed of the scanner in supine position (head first). The body and the head of the mouse were secured to the bed with tape.

### CT/PET Scanner, Data Acquisition and Reconstruction

CT and emission data were collected using a GE Healthcare eXplore VISTA dual-ring PET/CT scanner, housed in a temperature-controlled room. Thirty-six position-sensitive PMT detector modules and a dual layer phoswich detector technology provide high quality pre-clinical images throughout the field of view and the dual ring configuration which doubles the axial field of view and increases the sensitivity. A complete performance evaluation of the scanner has been done by Wang et al. (2006). A CT scan was obtained first for approximately 5 minutes (with a voltage of 40 kV and a beam current of 140 μA) followed by a 40 minute list-mode PET acquisition (with a 250-700 keV energy window), with the animal kept in the same position. The scanner has a ring diameter of 11.8 cm and a 4.8 cm axial field of view (FOV). 3-Dimensional (3D) sinograms were converted into 2-Dimensional (2D) sinograms before image reconstruction by Fourier rebinning. Images were then reconstructed by 2D-OSEM (two-dimensional ordered subset expectation maximization) reconstruction algorithm using the manufacturer's software. Corrections for random coincidence counts and photon scatter were applied.

### Image Analysis: Registration & Processing

All registration processing of the images to a standard template for voxel-based analysis were carried out using the Pmod suite of image processing tools (Pmod Technologies, CH). Prior to analysis the data were loaded into a database and the known shifts were applied to the PET data to bring it into alignment with the CT.

Data were Registered to the CT Image of the Digimouse Atlas

(http://neuroimaqe.usc.edu/Digimouse.html). Co-registration involved non-linear warping of the data to match the template image.

The higher resolution CT images were used for all of image registration steps. As CT data have a higher resolution and higher noise levels than the atlas image and are in Hounsfield units rather than the relative scale used by the atlas, we smoothed the images using a 3D Gaussian filter with FWHM of 0.5 mm in all directions. We also adjusted the dynamic range of the CT images to match that of the atlas, setting values below 700 HU to zero. Finally, truncation artefacts were removed from the CT images. A 3D region of interest that fully encompassed the head of the mouse was drawn on the images by hand and all voxels outside this region were set to zero.

To accurately align CT images to the Digimouse atlas images, manual alignment was initially achieved based on three orthogonal views through the CT images to determine rigid transformation parameters (i.e. shifts and rotations with respect to the three axes) that brought the images into rough alignment. Subsequently, the Brain Norm II algorithm was used to perform non-linear warping. This algorithm was designed for use with human brain images, but confirmed to work well with mouse pre-processed CT images, provided the size of the voxels was scaled by a factor of 10. Good fits and low residual errors were obtained in all cases.

For PET registration, the rigid and non-linear warping transformations calculated for the CT images (automatically aligned) were also applied to register images to the Digimouse template.

As voxel values in PET images are influenced by a number of factors (e.g. injected dose, weight of the animal, pharmacokinetics of the FDG), normalisation of the images is required. Thus, we selected the cerebellum (using the Digimous mask) as a reference region, as no significant changes are expected in brainstem areas due to the forebrain-specific promoter that drives transgene expression.

Once images were normalised to a standard atlas the Statistical Parametric Mapping package (SPM, Functional Imaging Lab, London, UK) was used to determine differences between groups (two sample t-test). Uncorrected SPMs (separately for areas of increase and decrease) were produced to show clusters of voxels with a statistically significant (p<0.05) difference between groups. The images were filtered to display only clusters of >10 voxels to reduce false positives. Regions of statistically significant increase and decrease were overlaid on a CT image for display using MRIcron (Rorden & Brett, 2000) and 3D rendered objects were produced using Pmod.

### Results

### PLB1 Animals: General Health and Appearance

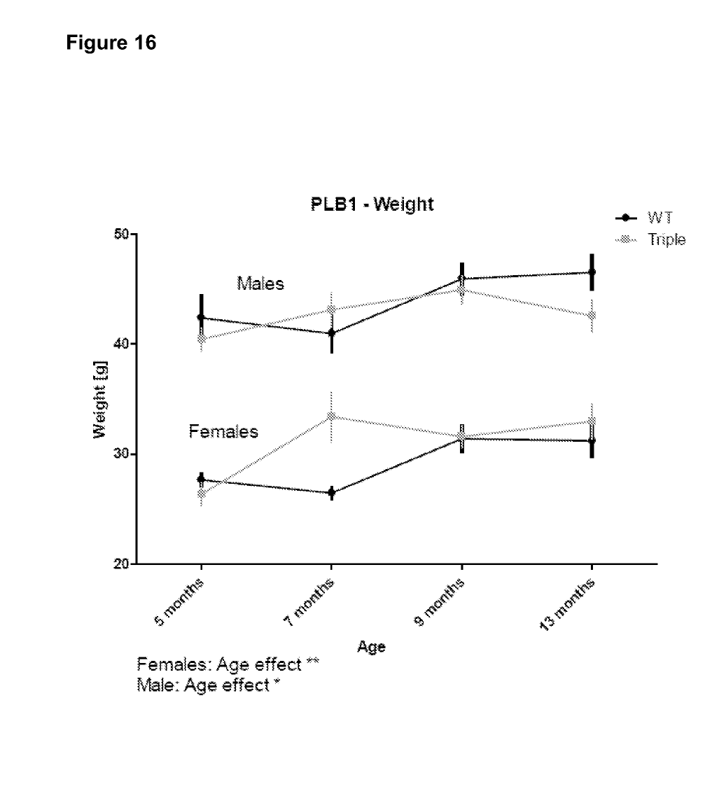

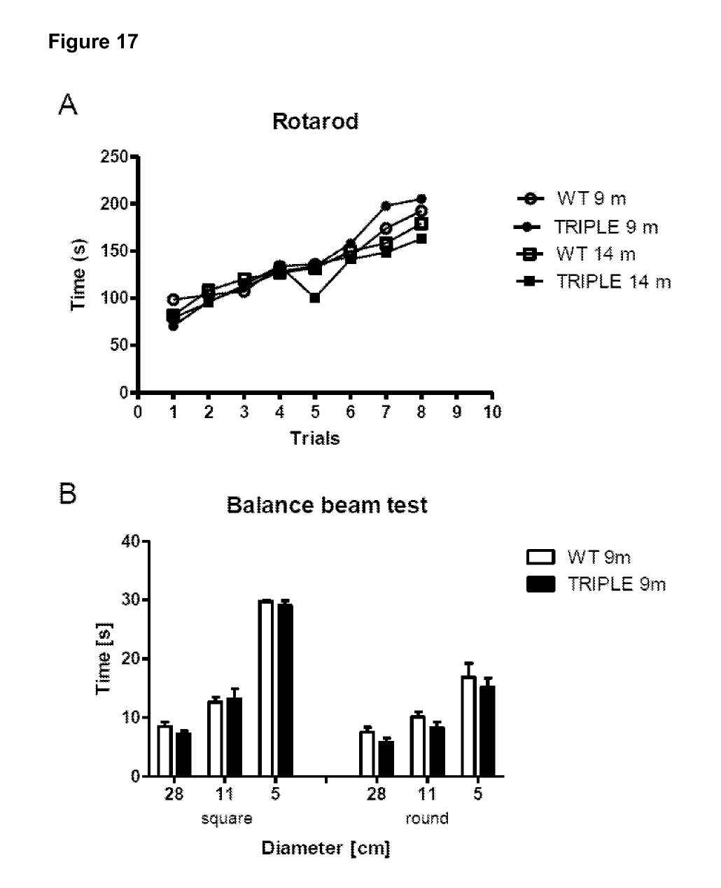

PLB1 Triple animals appeared of normal health, and overall growth did not vary between transgenic animals and WT littermates. A weight comparison showed a highly significant effect of gender (WT: [F(1,159)=140.7; p<0.001); Triples: [F(3,159)=100.9; p<0.001)]), and an age effect in males [F(3,135)=3; p<0.05)] and females [F(3,141)=4.49; p<0.01) of both genotypes (see FIG. 16). No genotype- or age-related deficits in motor learning were uncovered in the Rotarod or Balance Beam paradigms (see FIG. 17), indicative of intact sensory-motor coordination.

### Tissue Analyses

### Gene Expression

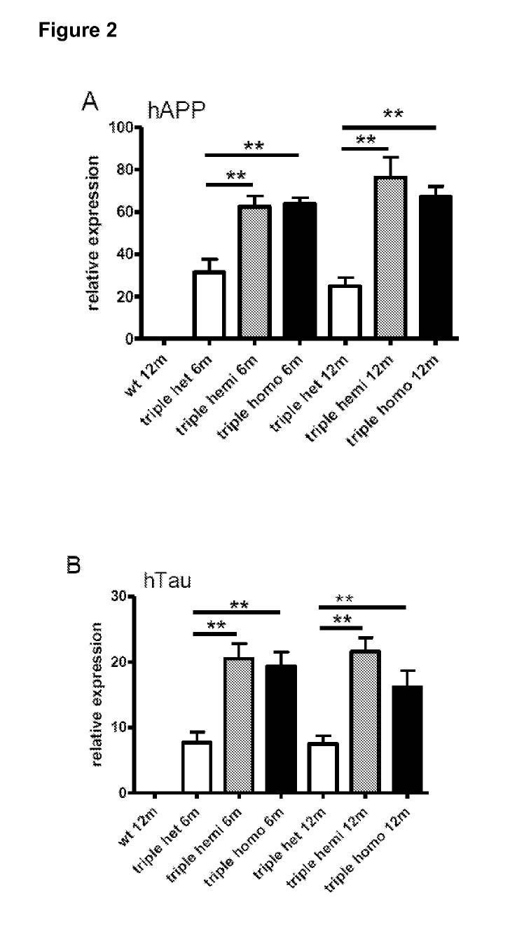

Due to the targeted knock-in procedure, PLB 1 Triple transgenic mice were expected to have stable and consistent gene expression. This was confirmed by quantitative real-time PCR (FIG. 2), since variability in gene expression between animals was found to be very low for both APP and tau. Both inserted transgenes were not significantly different between male (hemizygous) and female (homozygous) animals at 6 months or 12 months of age but about 2-3 fold lower in heterozygous females. Since absolute quantification was applied expression levels could also be compared between genes. This yielded about 3 fold less tau mRNA expression than APP. There was no expression in WT animals, additionally confirming primer specificity.

### Histology and Immunocytochemistry

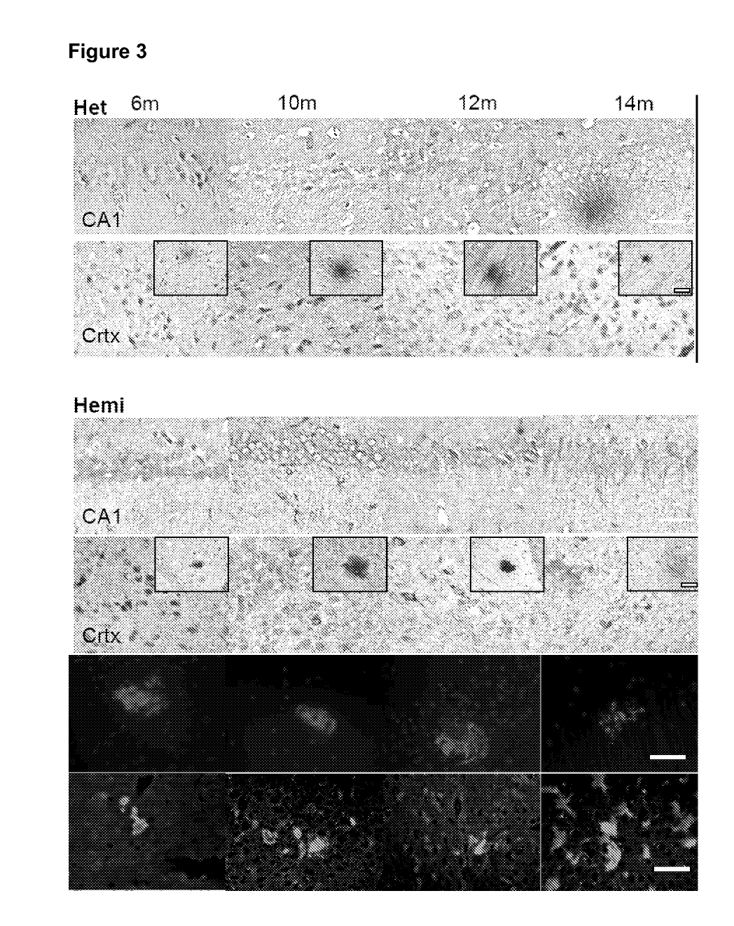

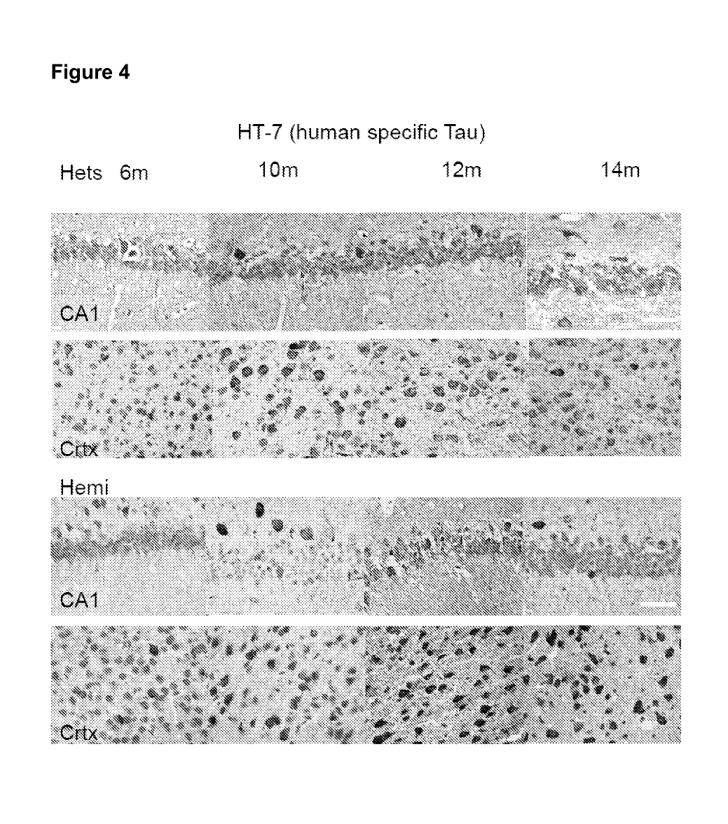

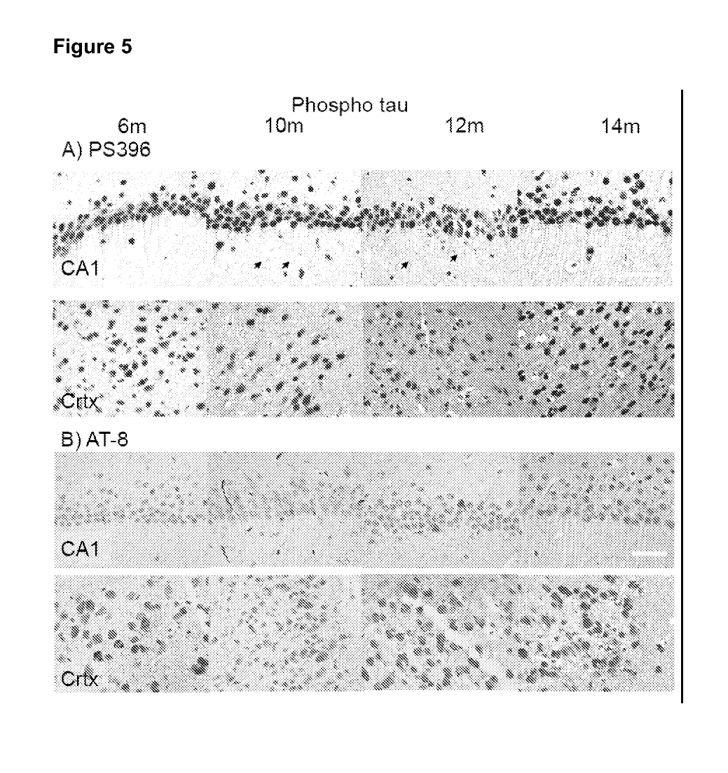

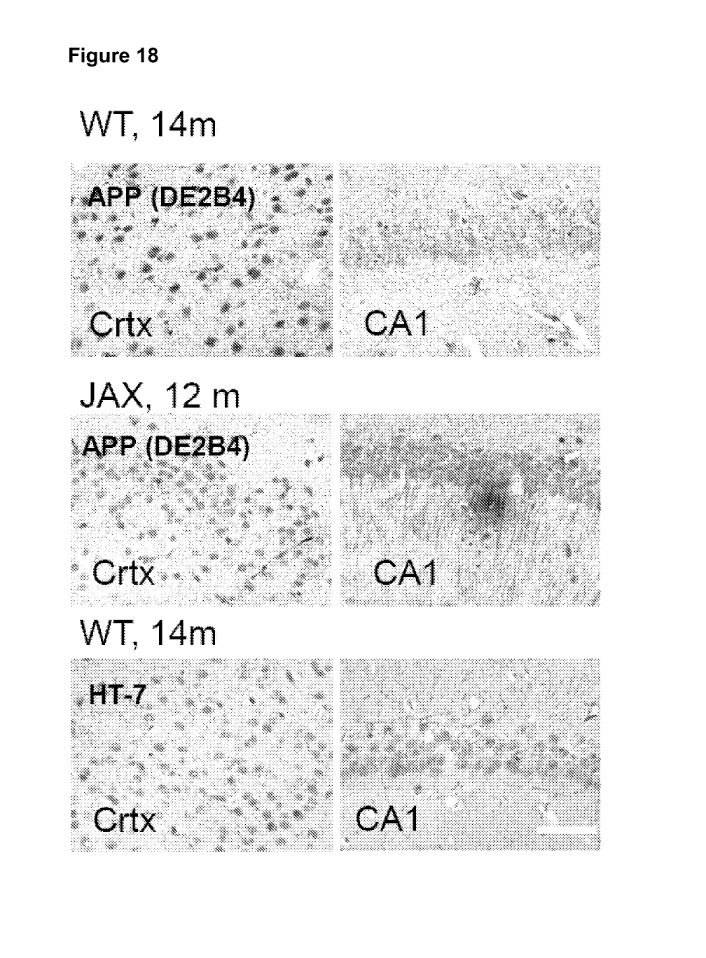

Detection of protein expression and pathology was determined in animals aged 6, 10, 12 and 14 months using human specific antibodies raised towards β-amyloid and tau (FIGS. 3 & 4, respectively. For WT, see FIG. 18). In both cases, immunoreactivity was more robust in samples from hemizygous compared to heterozygous animals reflecting the relative differences in gene expression. In PLB1 Triples, intracellular amyloid species were detected primarily within the soma of neurons from 6 months of age in hippocampal and cortical areas, though pronounced staining in the apical dendrites of the CA1 neurons was also found (FIG. 3). Independent from age, extracellular amyloid deposits were detected infrequently (<6 per section). For detection of diffuse plaques, 13-sheet aggregation was also determined by Congo Red and Thioflavin-S staining (FIG. 3). Such amyloid immunoreactivity was absent in wild type littermates but closely matched the one present in Jax mice over-expressing hAPP (see FIG. 18). Tau reactivity (HT-7) was primarily confined to Triples, though a subtle cross-reactivity with endogenous tau was evident in PLB1 WT littermates (FIGS. 4 & 18). PLB Triples demonstrated a high number of hTau positive neurons present throughout the forebrain, particularly distinct in cortex. This was most pronounced in hemizygous Triples even at 6 months, with evidence of an age dependent loss of hTau from neurites seen in conjunction with the somatic accumulation of tau (FIG. 4). A similar shift in distribution was seen when visualising PS396 but not AT8 phospho-tau species in hemizygous animals. Distinct from PS396, AT8 phospho-tau was evident in cortex across all age groups, whilst only limited AT8 immunoreactivity was present in the hippocampus, confined to the somata of 12-14 m PLB1 triple animals (FIG. 5).

### Synaptic Transmission and Plasticity in Hippocampal Slices

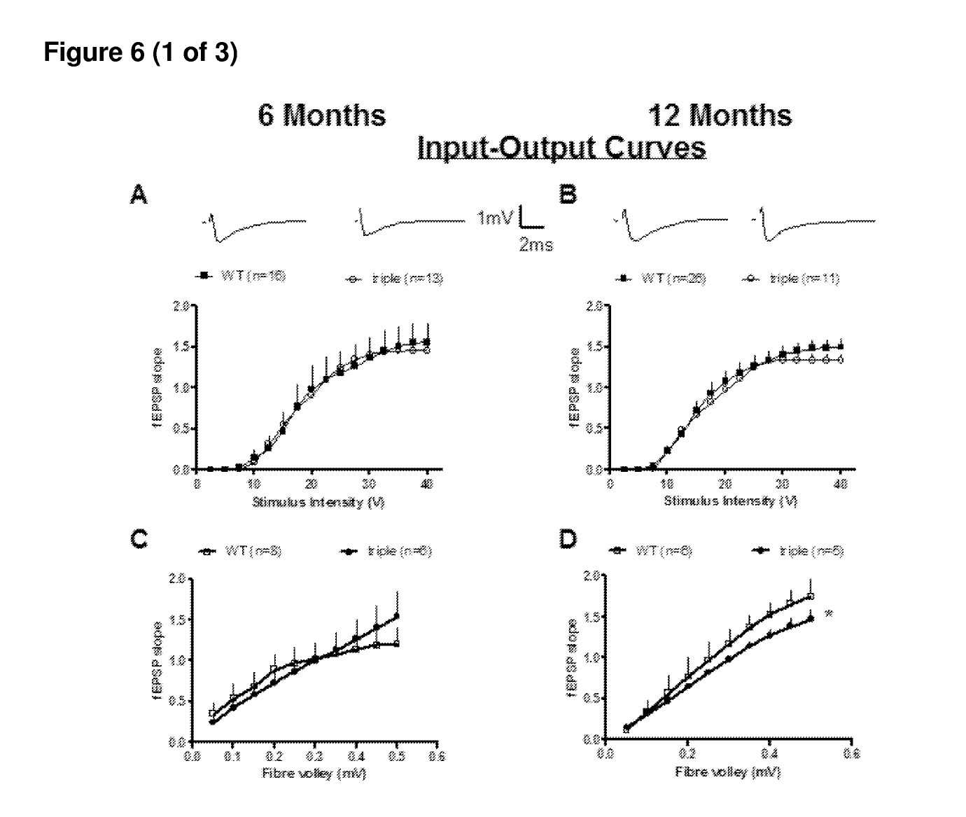

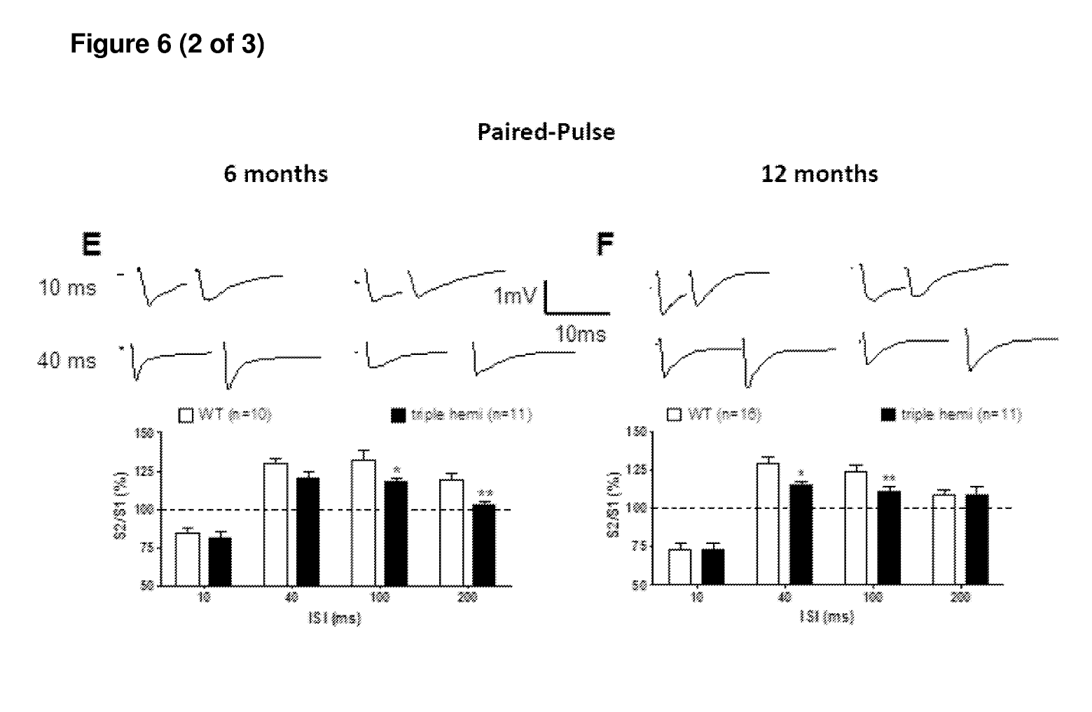

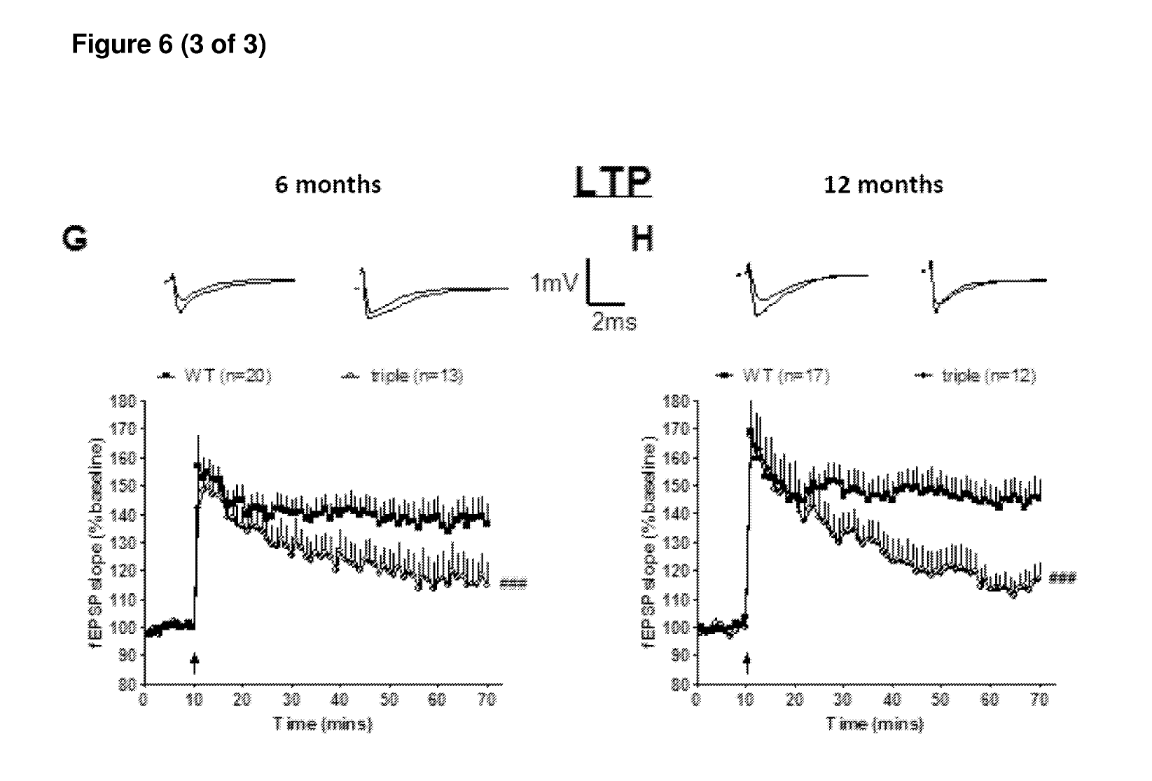

Initial experiments characterised electrophysiologically, the properties of hippocampal slices from the PSEN homozygous mice vs. WT mice at ages 6 and 12 months, as this line was used in the generation of the PLB1 triple transgenic mice. No significant differences were observed in 10 curves, paired-pulse responses or LTP in slices from PSEN mice compared to PLB1 wild-type mice (data not shown); therefore, data obtained from PLB1 triple transgenic slices were compared here solely to PLB1 WT data. Subsequently, 6- and 12-month PLB1ₜᵣᵢₚₗₑ mice (males only) were compared with wild-type PLB1 mice. Basic synaptic transmission parameters were established for fEPSPs slopes plotted against stimulus intensity and presynaptic fibre volleys (FIG. 6A-D). 10 curves of fEPSP slope against stimulus intensity were not significantly different between PLB1ₜᵣᵢₚₗₑ and wild-type mice at either age group (all P′ s>0.05). However, the intensity of the stimulus applied to the Schaffer collaterals gives an inaccurate measure of the levels of afferent activation. Therefore, responses were also analysed for fibre volley amplitude. Although no deficits were evident in the PLB1ₜᵣᵢₚₗₑ group at 6 months of age, the fEPSP slope vs fibre volley amplitude 10 curve of the transgenic group was statistically different to WT at 12 months (genotype effect: P<0.05). These results indicate that basic synaptic transmission was impaired in the PLB1ₜᵣᵢₚₗₑ group at 12 months of age.

In order to measure short-term plasticity, slices were stimulated with pairs of pulses 10, 40, 100 and 200 msec apart and the slope of the second fEPSP was expressed as a percentage of the first (FIG. 6E-F). For the ISI of 10 msec, paired pulse inhibition (PPD) was observed, while ISIs of 40, 100 and 200 msec resulted in paired pulse facilitation (PPF). At both 6 and 12 months of age there were no significant differences in the levels of PPD between wild-type and transgenic mice (all P′ s>0.05). However, a significant reduction in PPF was discovered in slices from PLB1ₜᵣᵢₚₗₑ mice. At 6 months PPF was significantly reduced for all ISIs >40 (100 msec: P<0.05; 200 msec: P<0.01). Similarly, at 12 months, levels of PPF was reduced for ISIs of 40 (P<0.05) and 100 msec (P<0.01). Theta-burst LTP was also investigated in the CA1 region of hippocampal slices (FIG. 6G-H). A significant deficit in LTP was found in PLB1ₜᵣᵢₚₗₑ mice at both age groups. A two-way repeated measure ANOVA revealed a significant genotype×time interaction (P<0.05 at 6 months; P<0.0001 at 12 months), indicative of a time-dependent effect. At 6 months, although short term potentiation was induced it declined considerably to 115±8.4% of baseline at t=70 min, compared to 136±7.7% in WT slices. Similarly, at 12 months, LTP was not stably induced in slices from triple transgenic mice, decreasing to 117±5.7% of baseline at t=70 min compared to 146±6.4% in WTs.

### Circadian Rhythms, Vigilance States and Global Brain Activity (EEG)

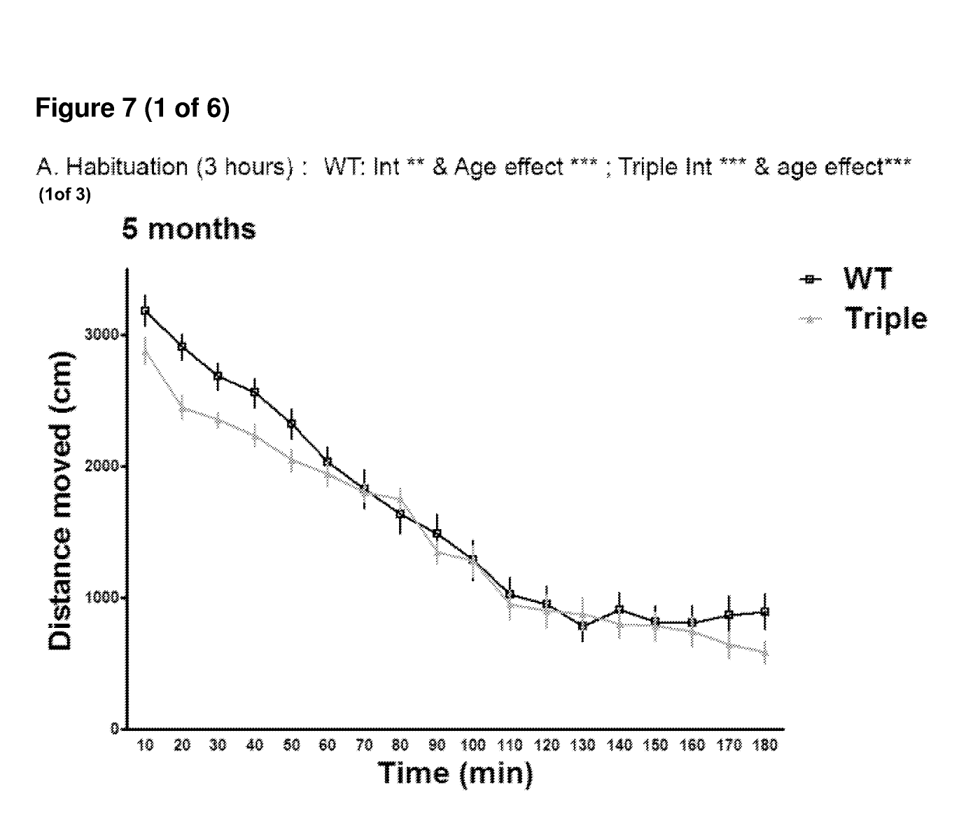

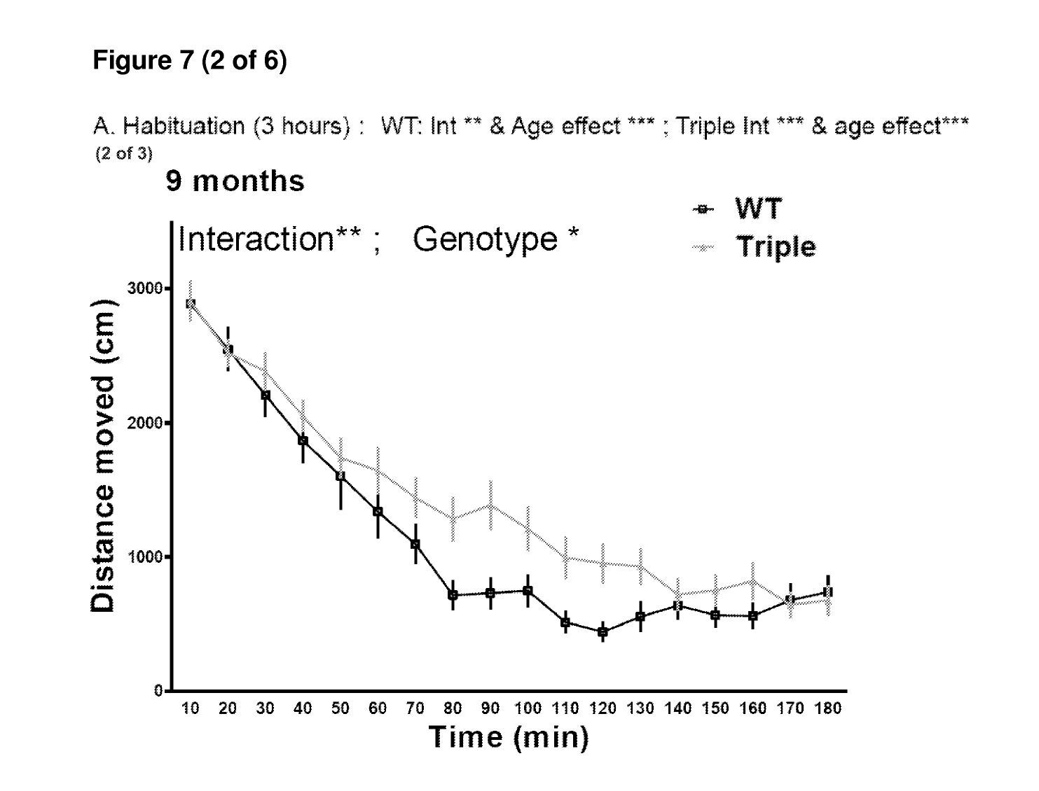

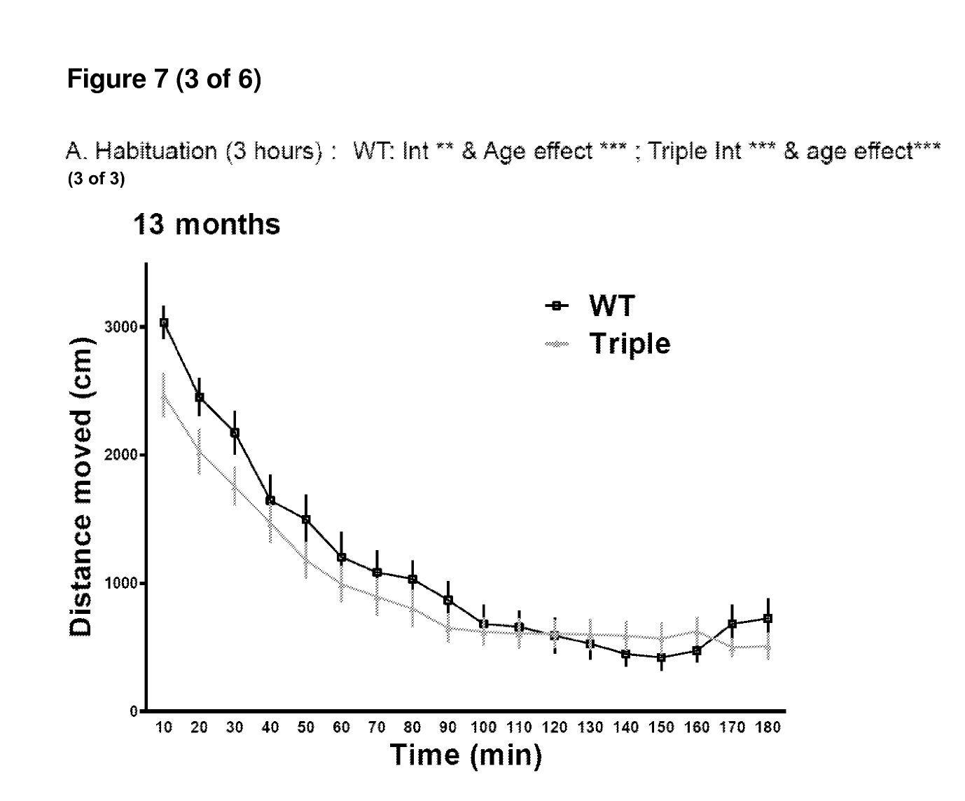

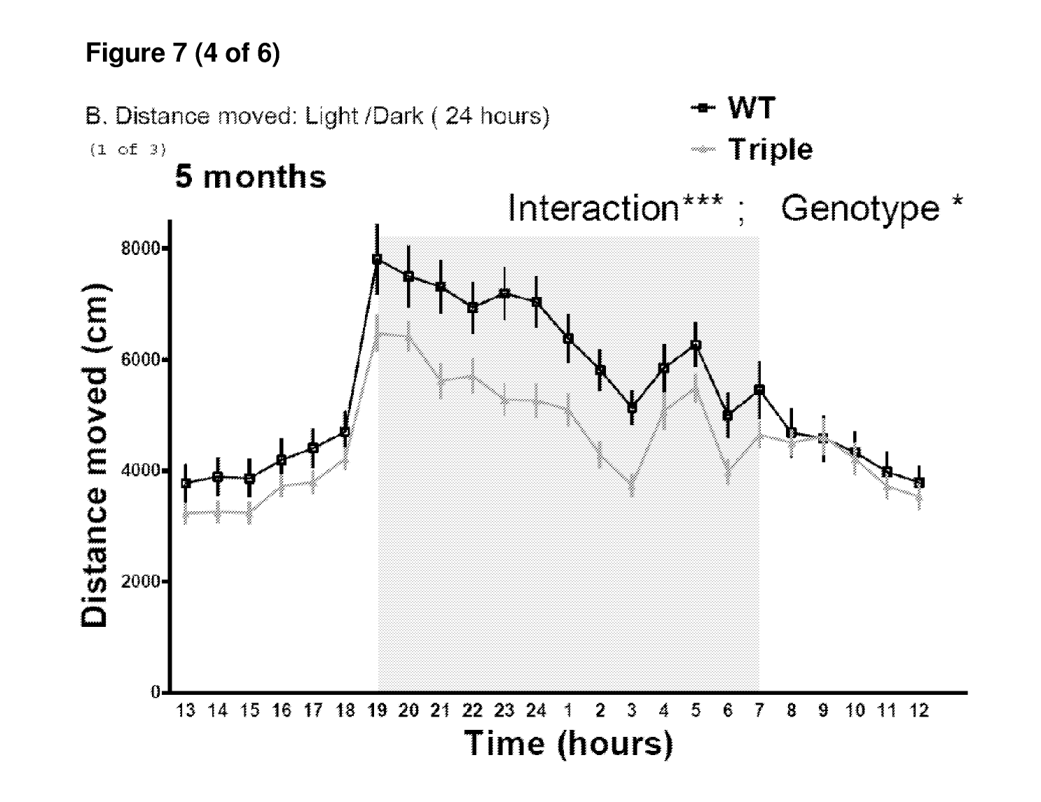

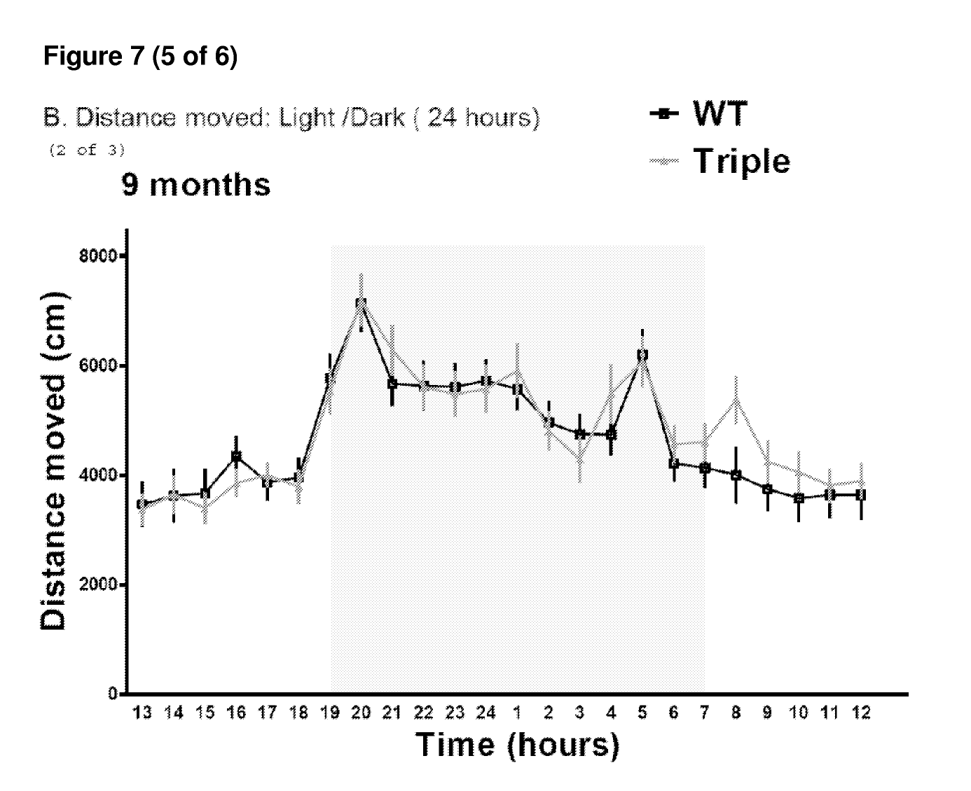

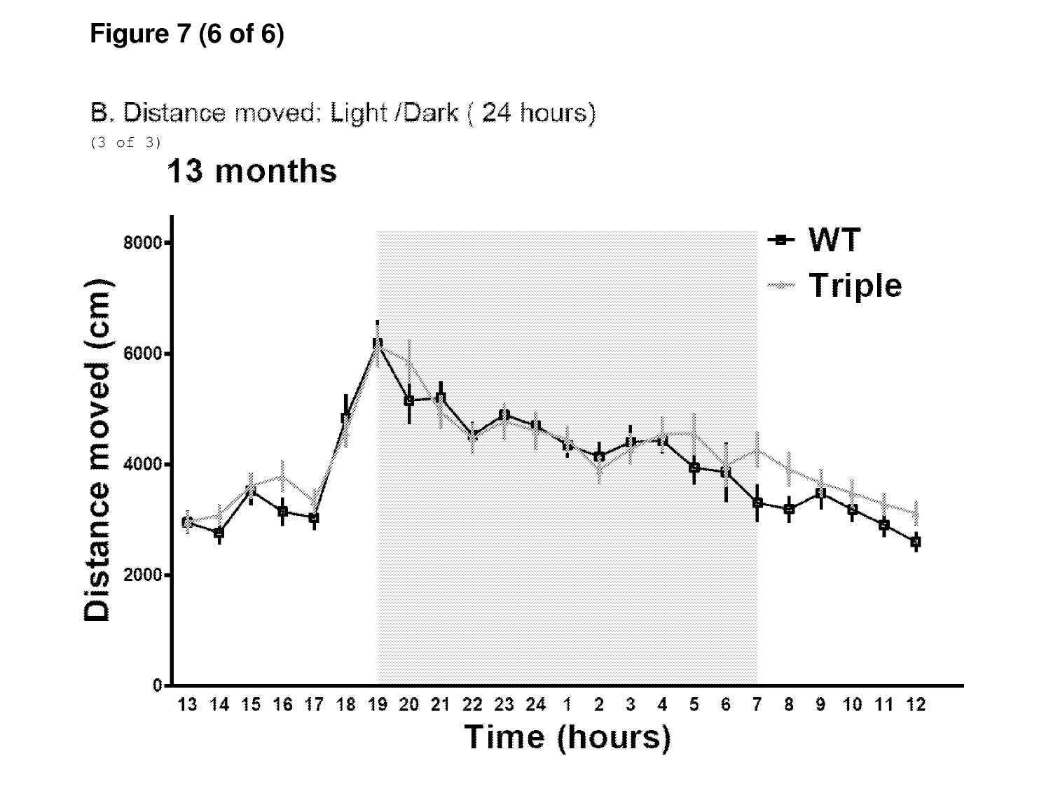

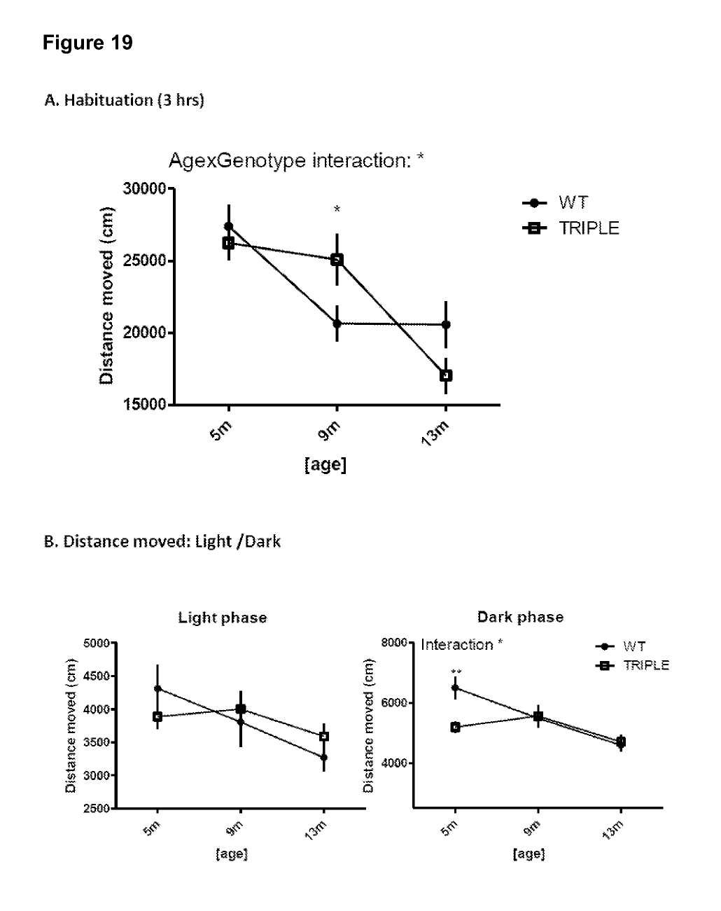

Circadian locomotor activity was determined in the PhenoTyper system for both PLB1 Triples and WT at the age of 5, 9 and 13 months. During habituation, all groups of animals reduced their activity (distance moved) over the first 3 hours. A significant age and genotype effect was revealed (genotype×age interaction, F(2,150)=3.50; p<0.05; FIG. 7A & FIG. 19). In WT, a significant reduction of distance moved occurred between 5 and 9 month of age, whereas in PLB1 Triples a significant decline was evident at a later age, i.e. between 9 and 13 months. As a result, WT and Triples differed significantly at 9 months (FIGS. 7A & S6A). This signifies an age- and genotype dependent alteration in habituation to a novel environment.

The overall distance moved recorded over five days (excluding 2 days of habituation, FIG. 7B) of light and dark suggest a significant age effect in WT [(F(2,71)=5.39 p<0.01]. An interaction between age and light vs. dark phase [(F(2,71)=4.71 p<0.01] was evident with PLB1 Triples at 5 months exhibiting significantly lower activity compared to age-matched WTs during the dark phase (see also Fig. S6B).

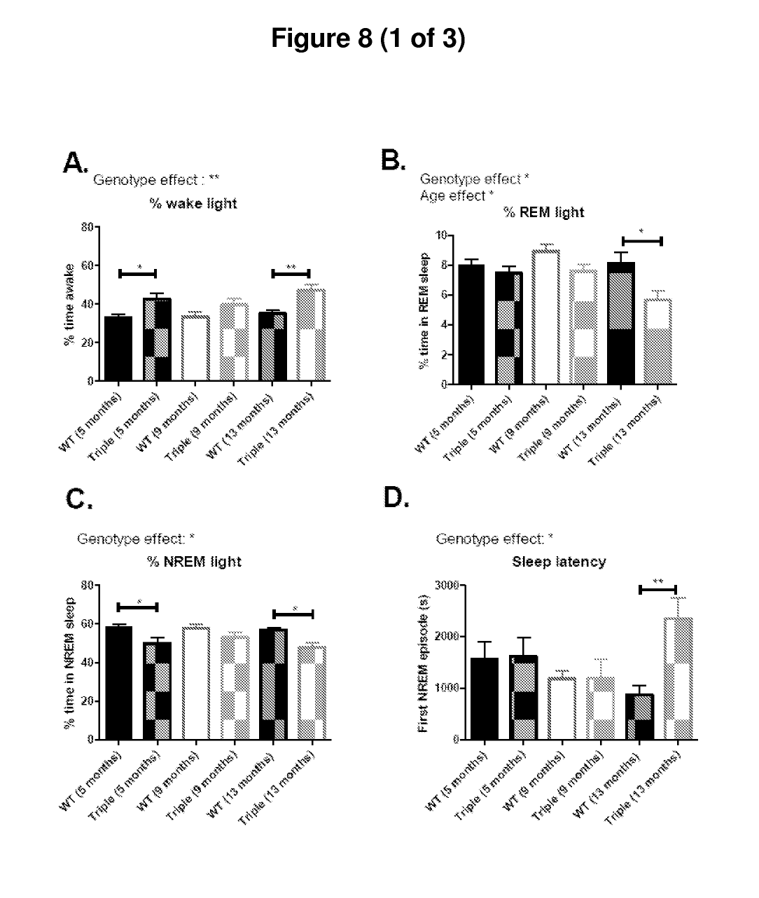

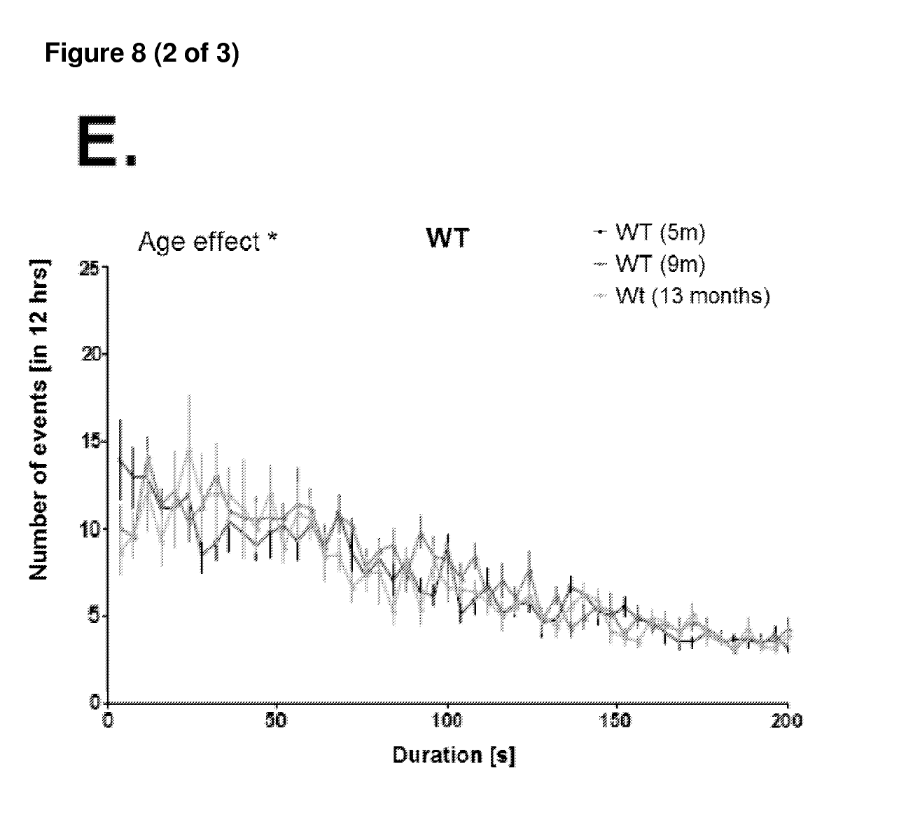

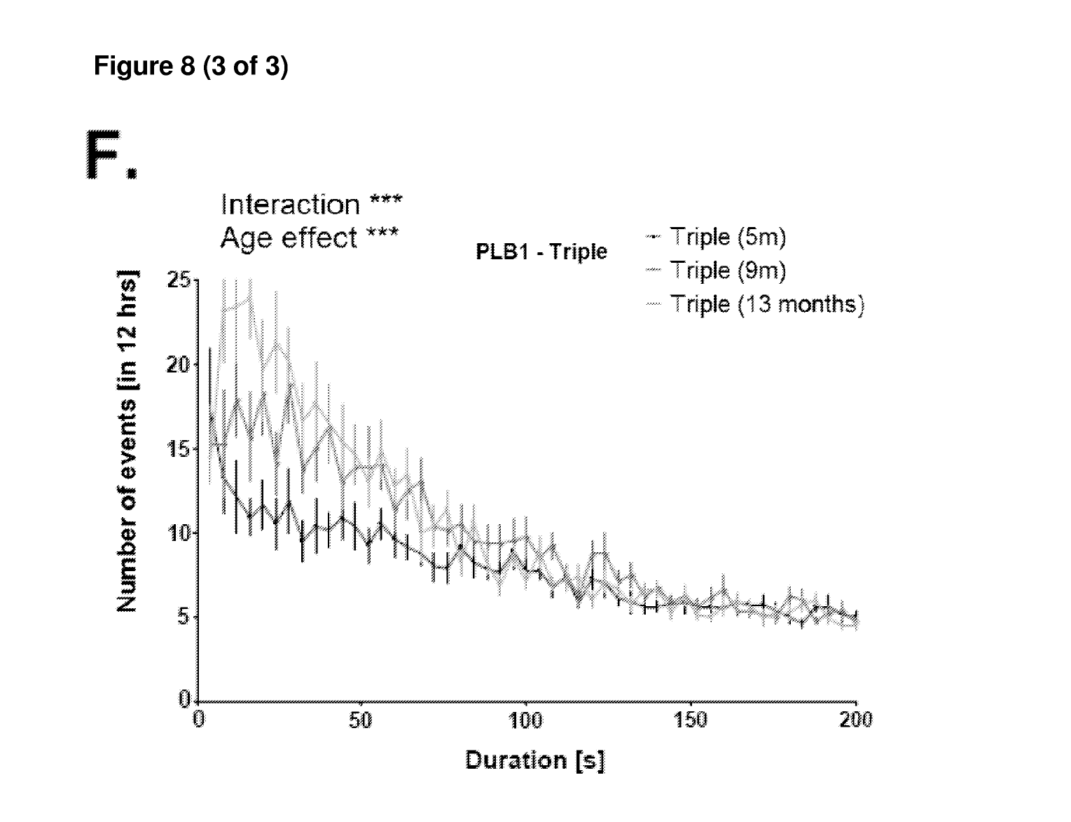

Analyses of EEG- and activity guided vigilance state classification during the light phase suggested that PLB1 Triple animals had a significant genotype effect in all stages (FIG. 8, A-C). They spent significantly more time awake compared to WT (overall effect of genotype: F(1,30)=11.22; p<0.01)). Paired comparison confirmed this to be significant at 5 and 13 months (p<0.01) (FIG. 8 A). In agreement with this, the onset of first sleep occurrence (i.e. latency to 1st NREM episode) was delayed in PLB1 triples at the latter age group (FIG. 8D, p<0.01). Furthermore, genotype effects obtained during both sleep phases signified reduced REM (FIG. 8B) and NREM (FIG. 8C) sleep in Triples (REM: age effect [F (2,28)=4.69; p<0.05); genotype effect [F (2,28)=6; p<0.05); NREM: genotype effect [F (2,28)=2.12; p<0.05)].

Over all groups, REM sleep was affected by both age and genotype. Paired comparisons confirmed significant differences in REM for the 13 months age group (p<0.05), and a decrease in NREM sleep at 5 (p<0.05) and 13 months (p<0.05) in Triples compared to age-matched WTs.

Sleep disturbance and fragmentation in PLB1 Triples was further indicated by an analysis of the relative occurrence and distribution of NREM sleep episodes (frequency vs. duration of NREM events): The distribution of NREM events in triples displayed a significant increase in short-NREM events (age effect: F (2,1050)=44.99 p<0.001] and interaction (age×NREM duration): F (98,1050)=2.5 p<0.001; FIG. 8F), while WT showed only an age effect [F (2,1050)=2.5 p<0.05] (FIG. 8E).

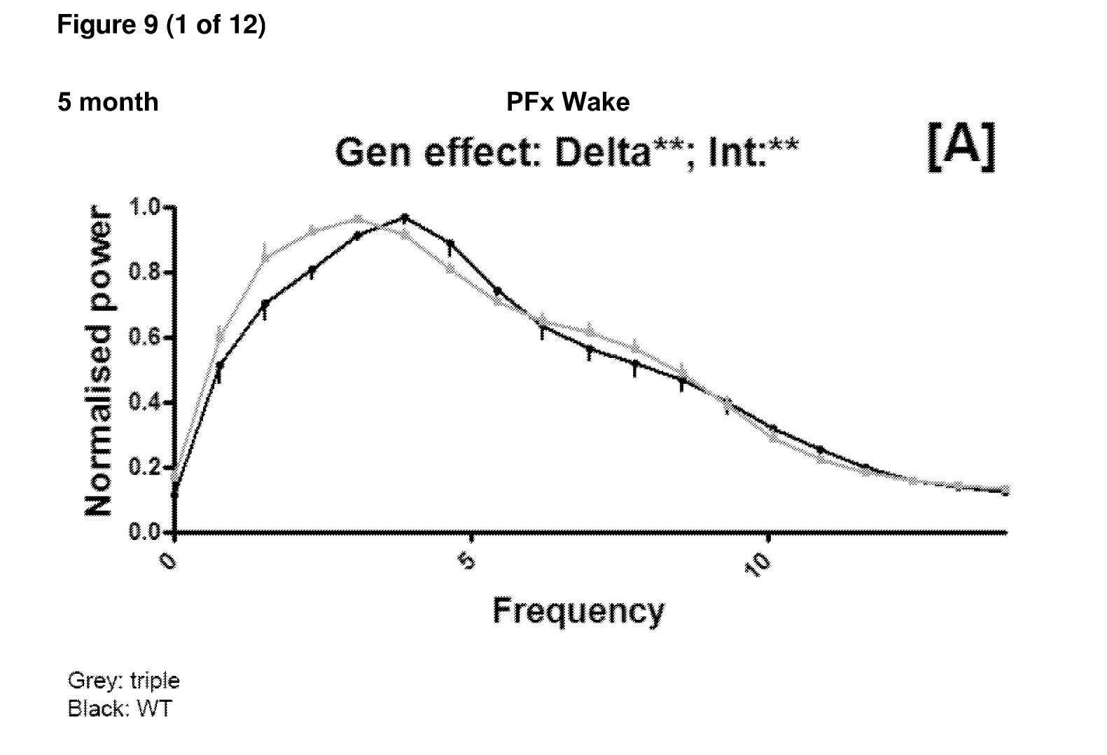

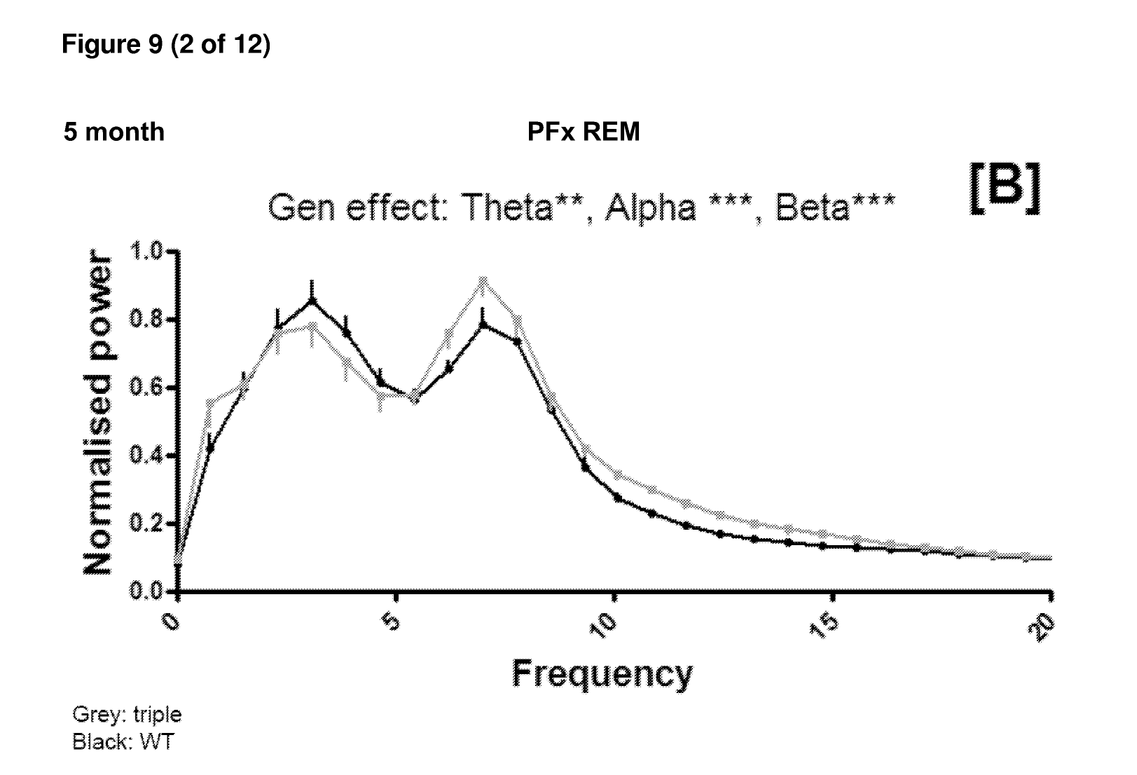

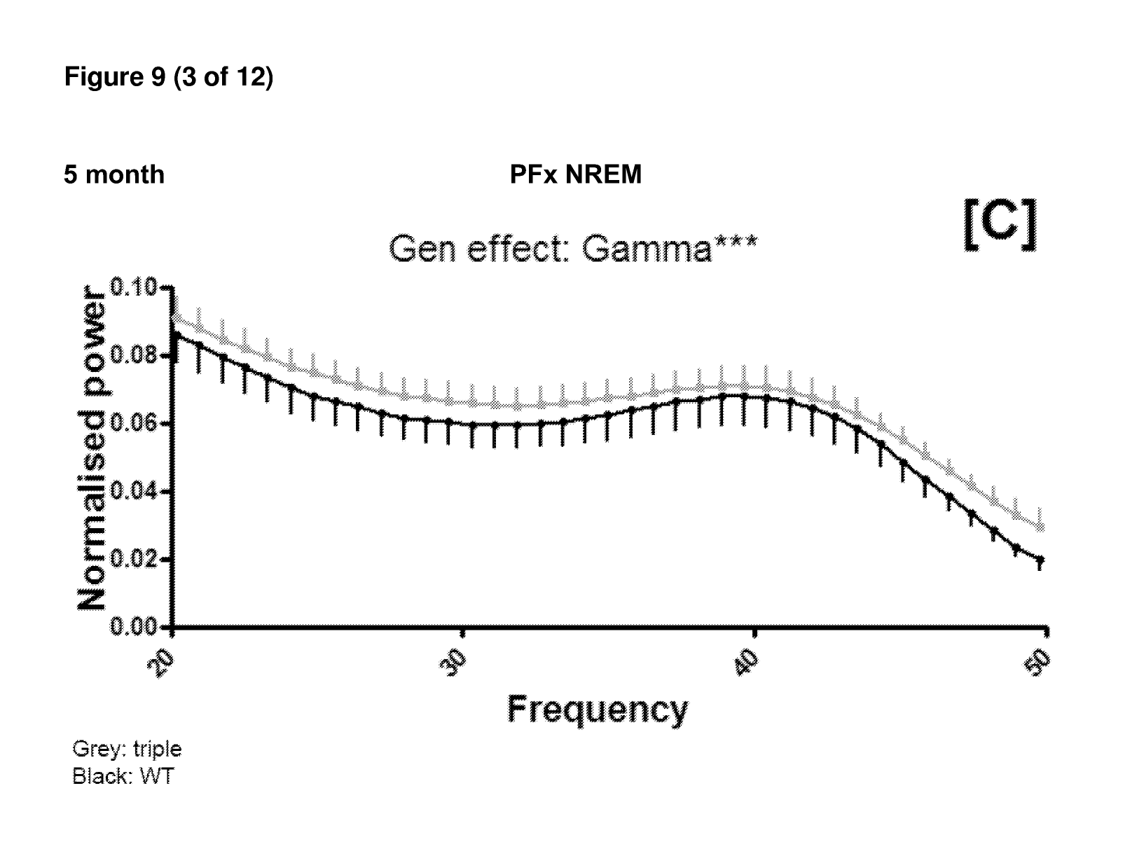

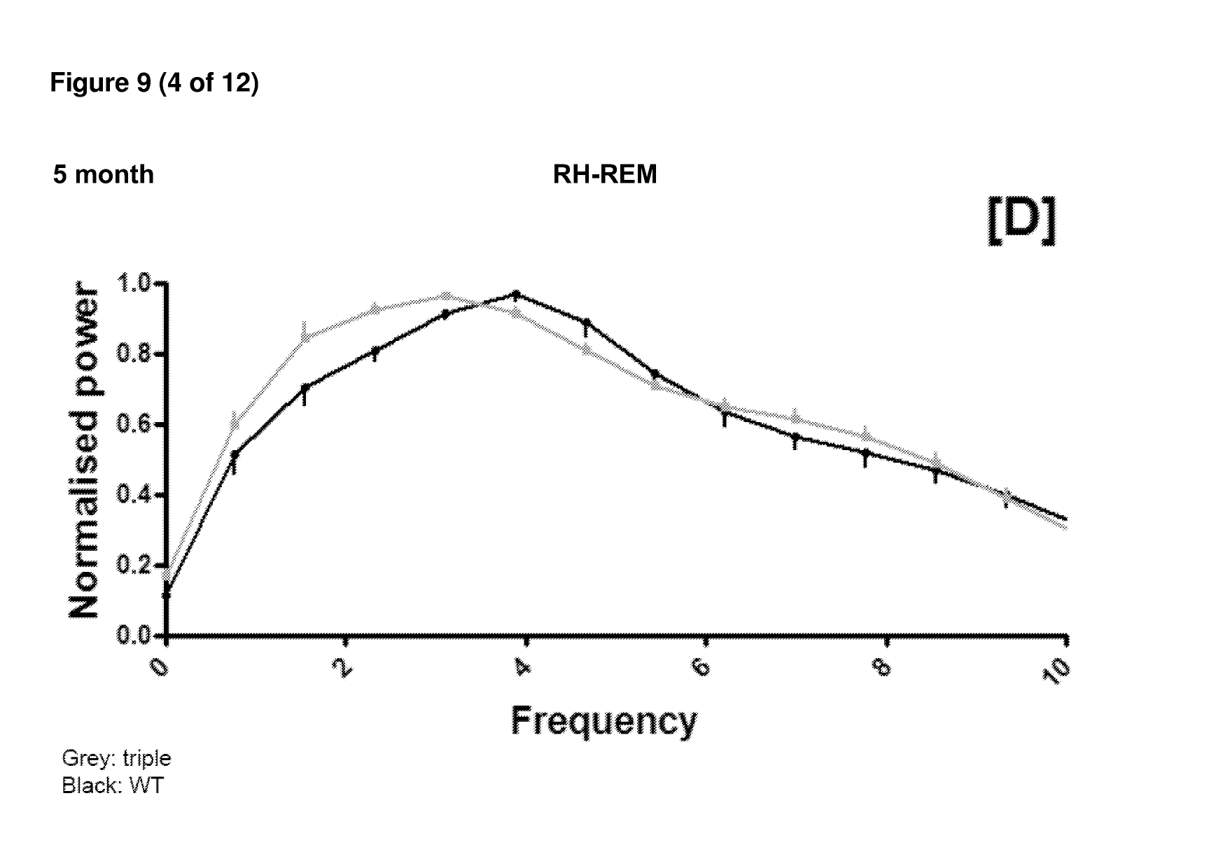

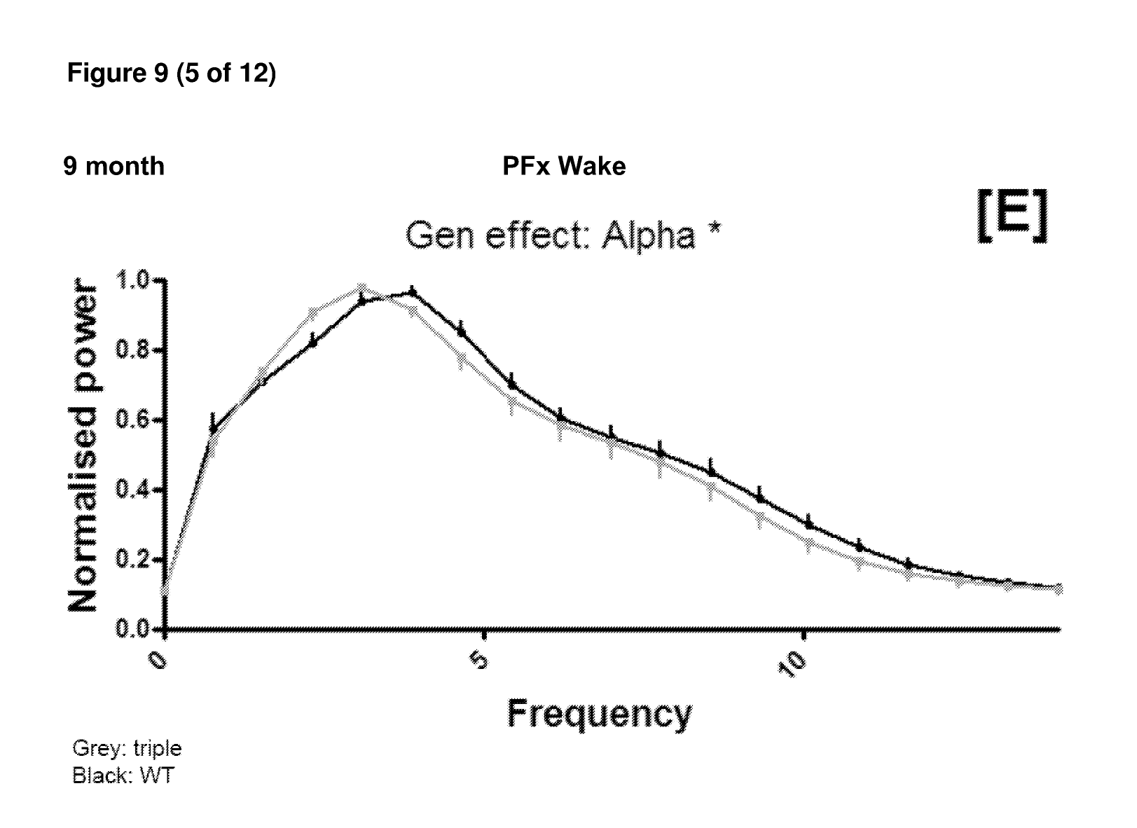

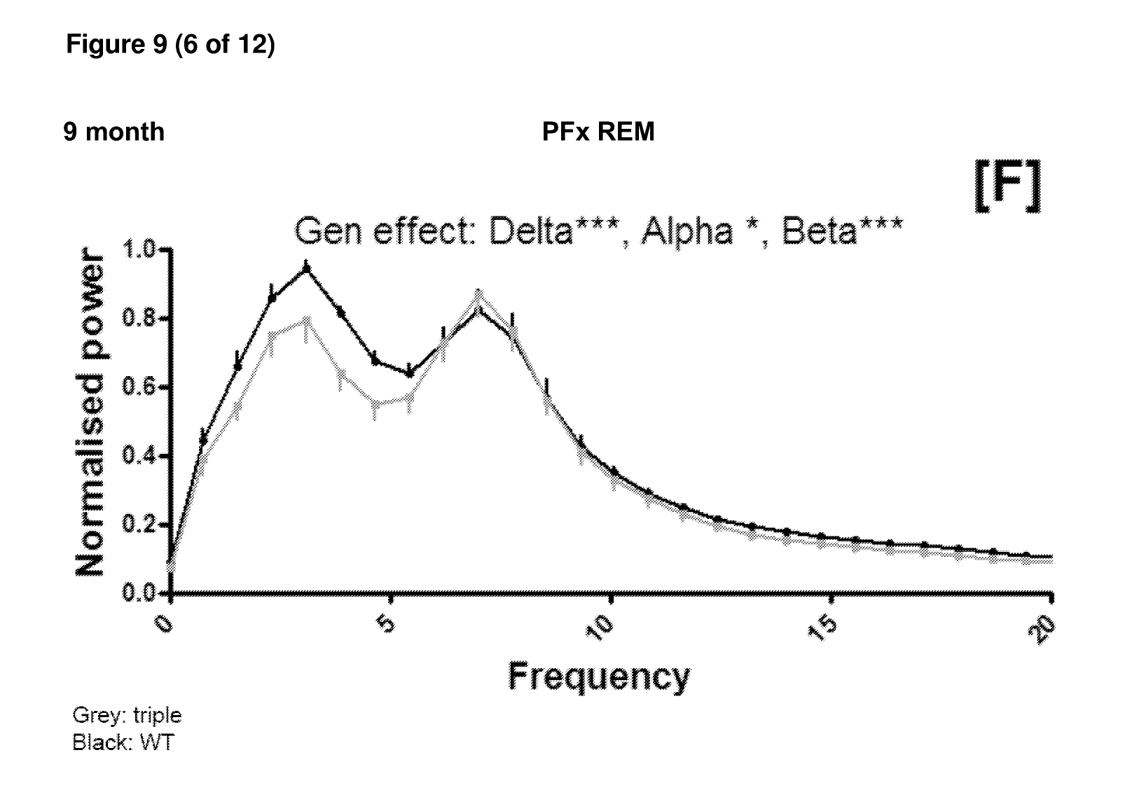

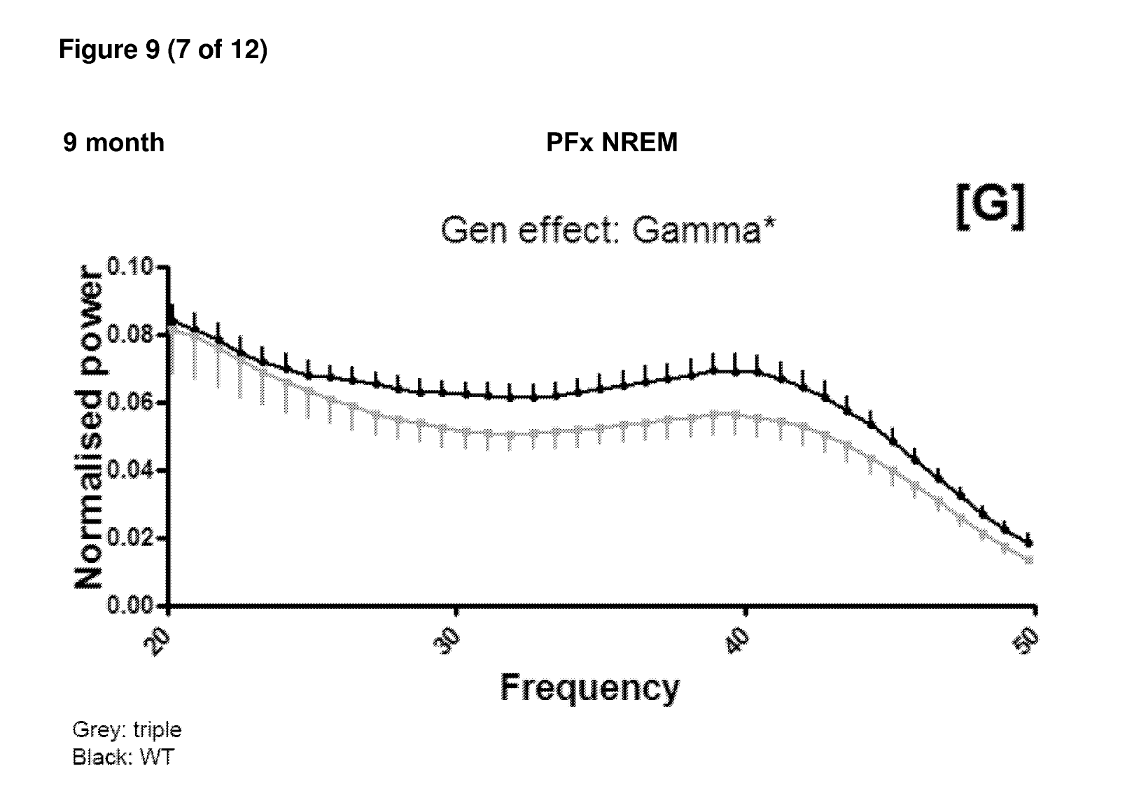

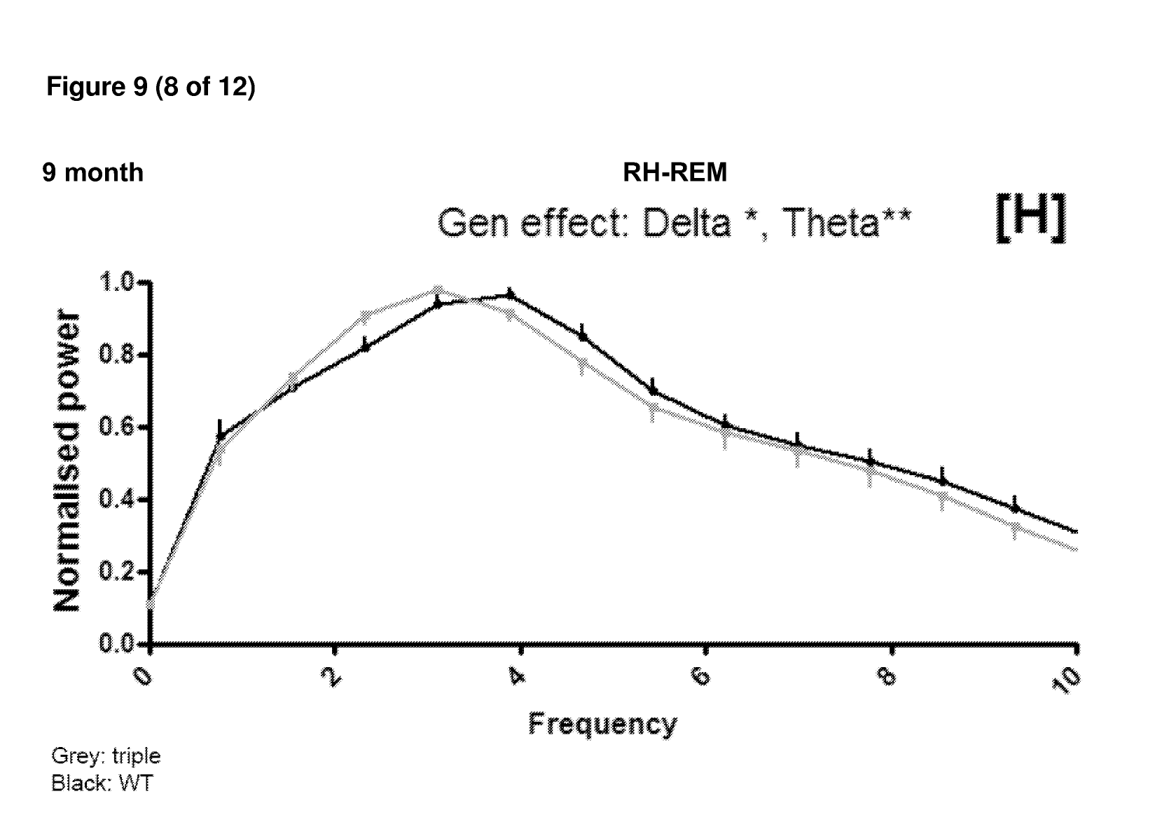

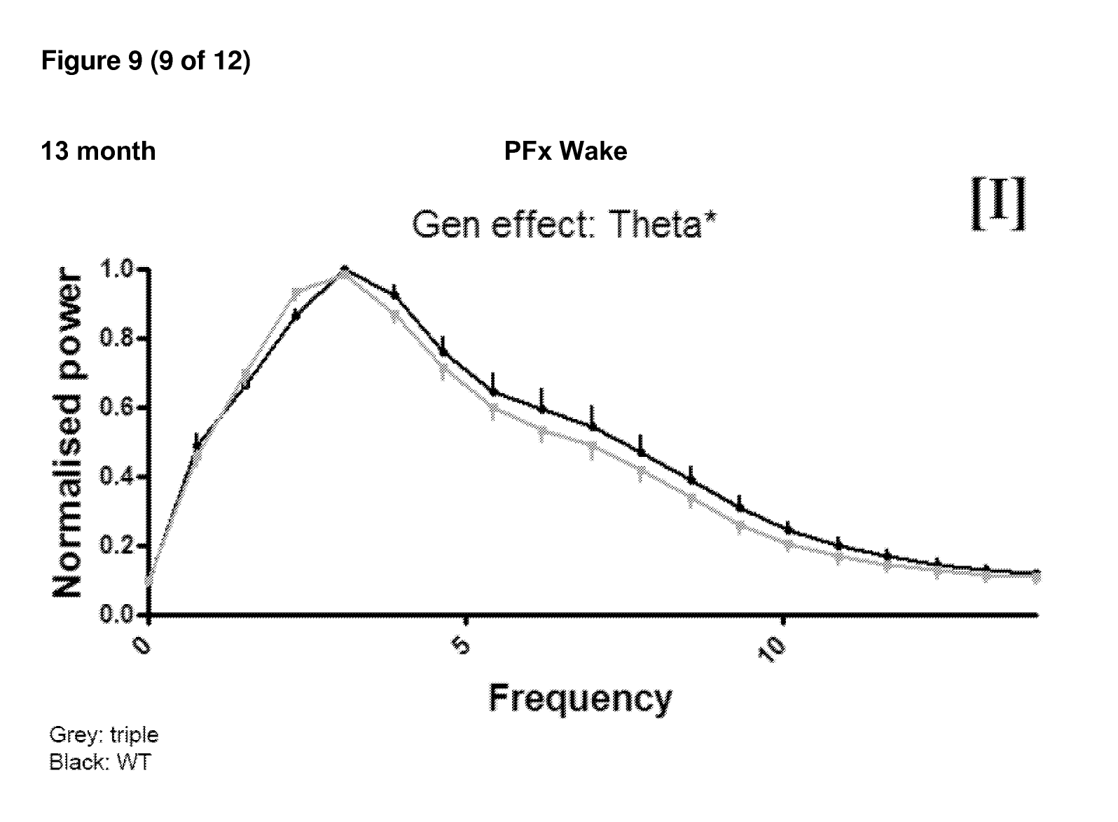

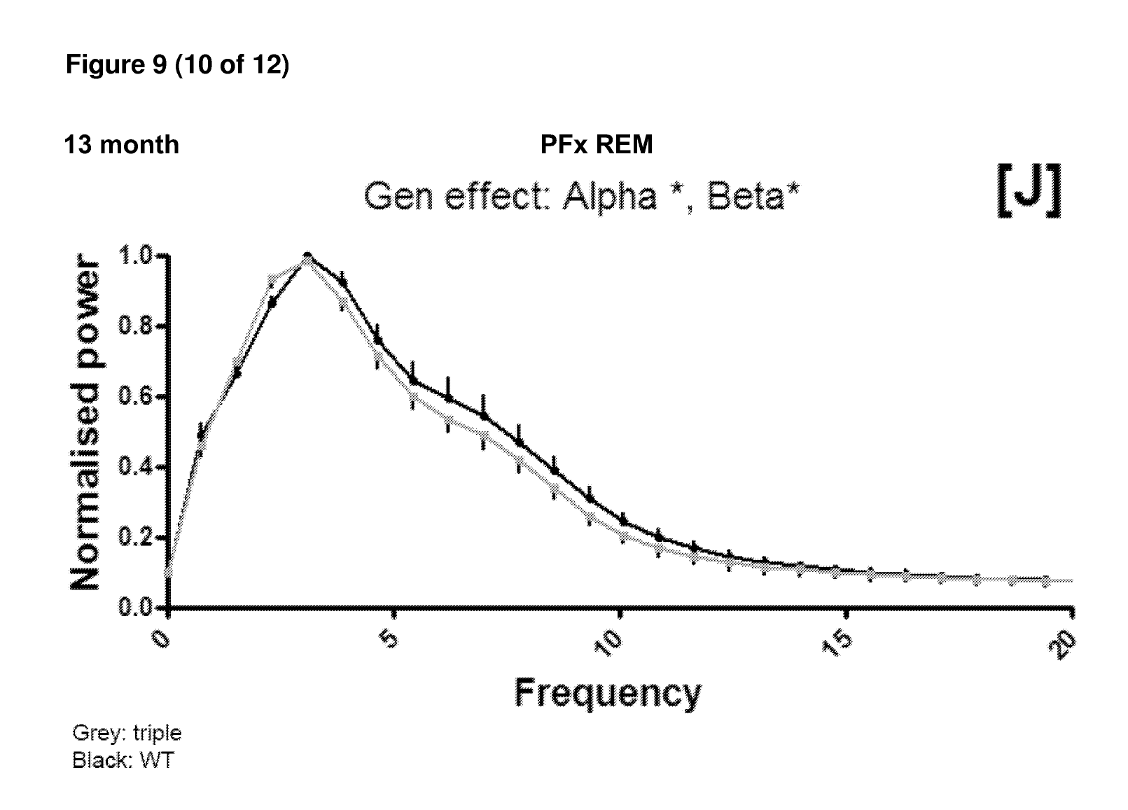

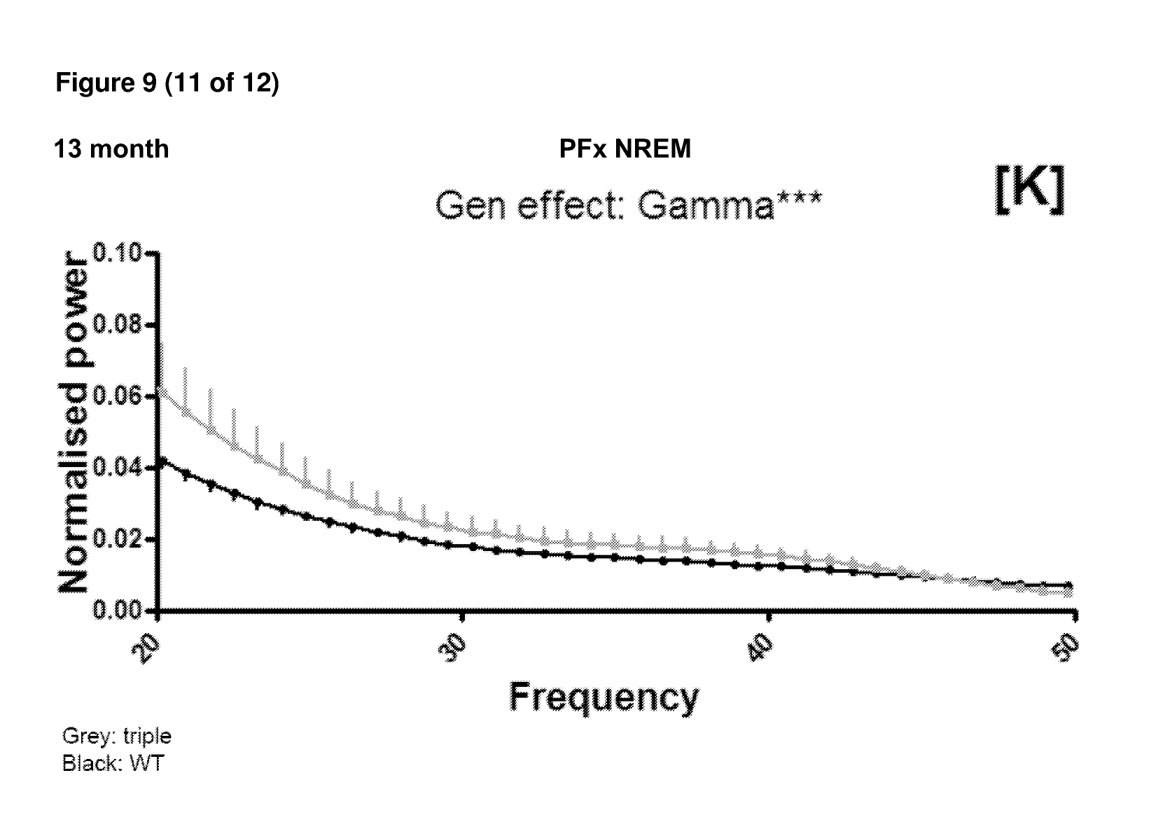

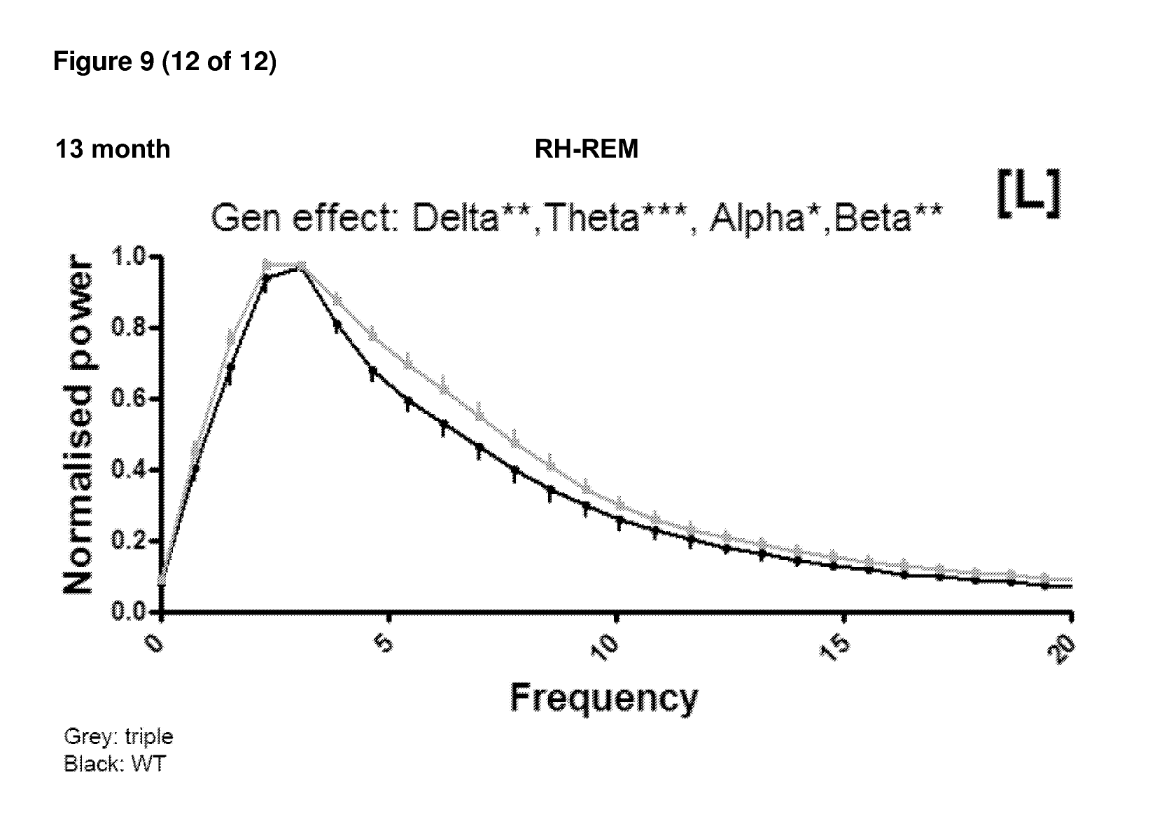

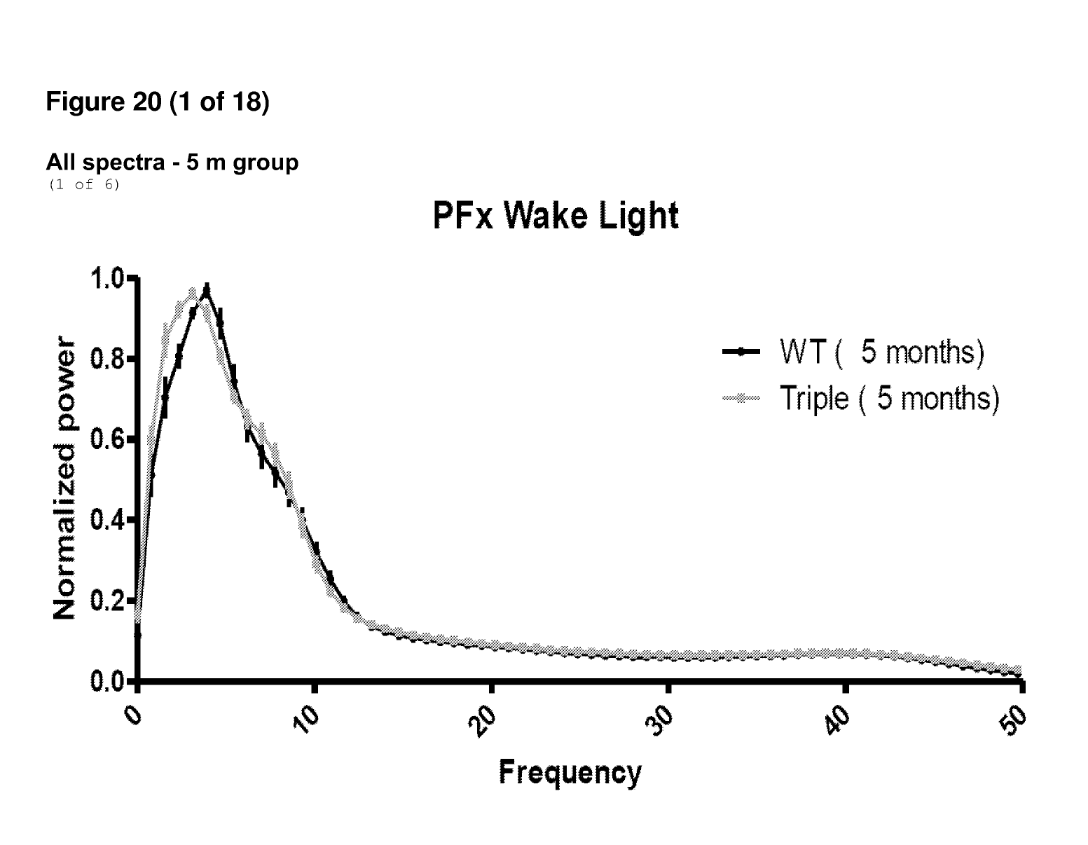

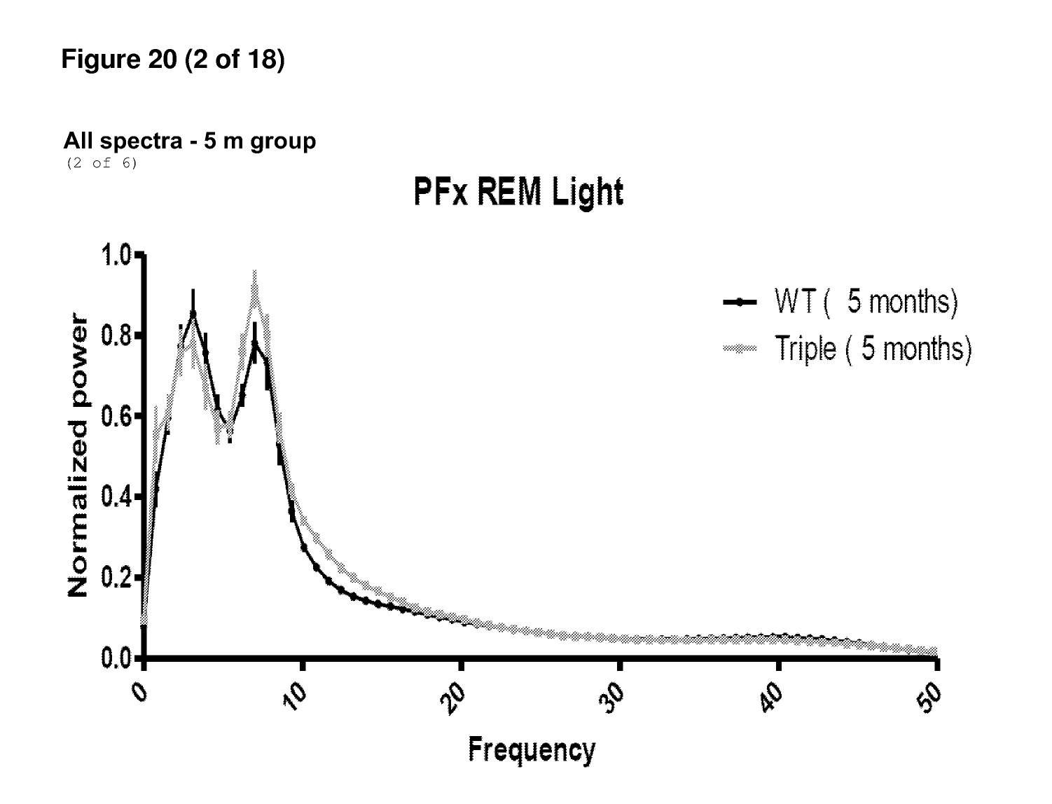

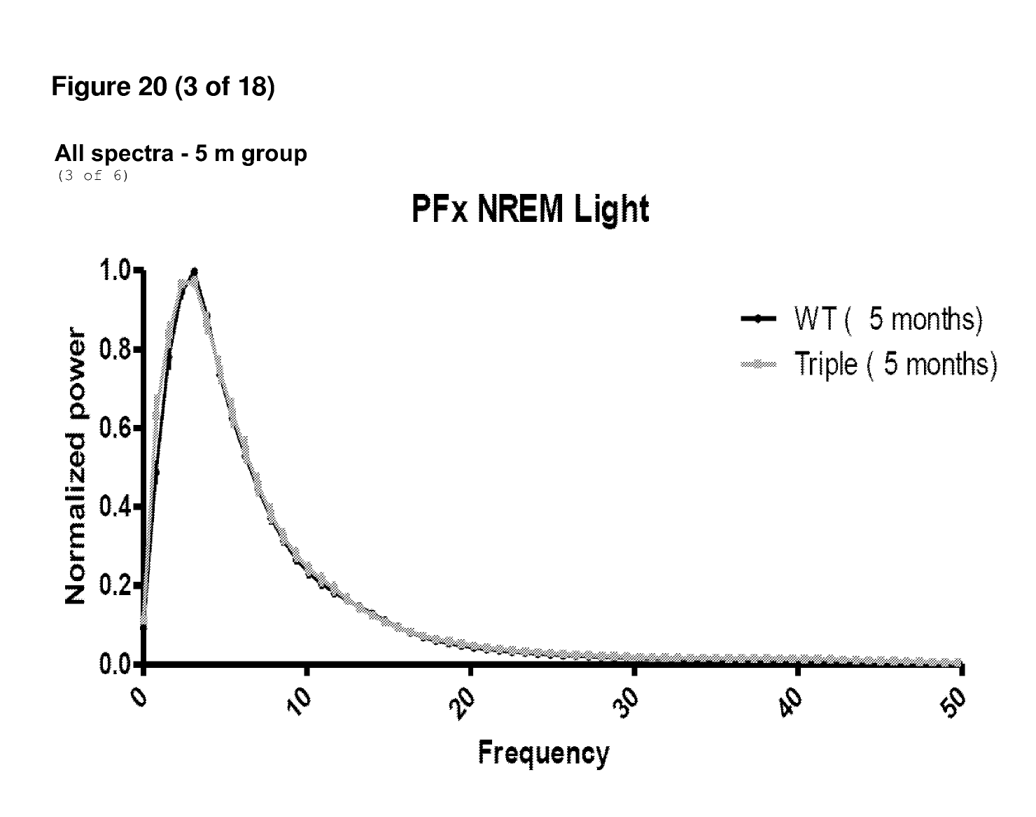

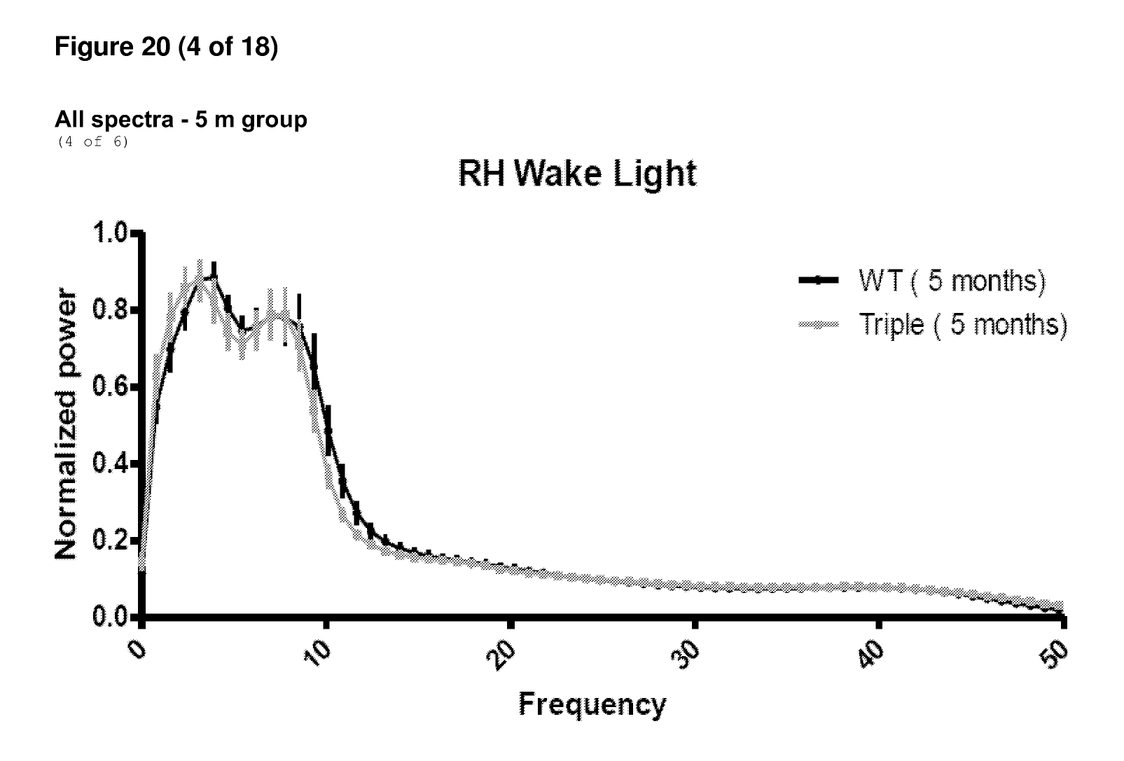

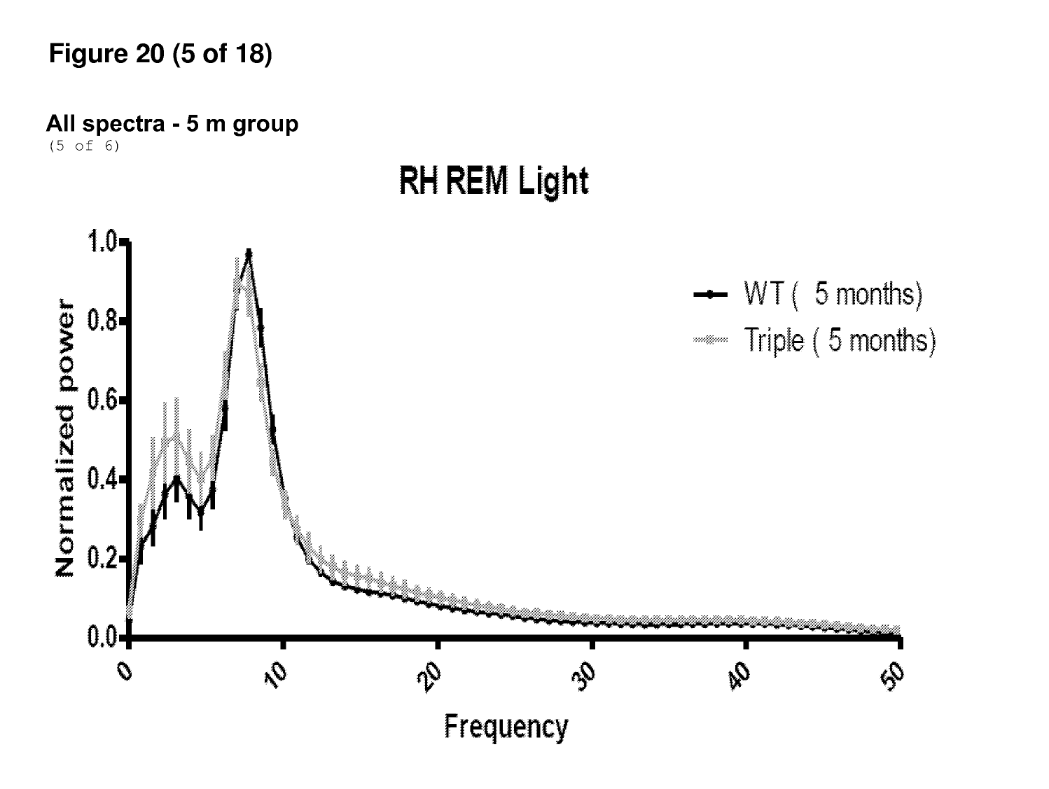

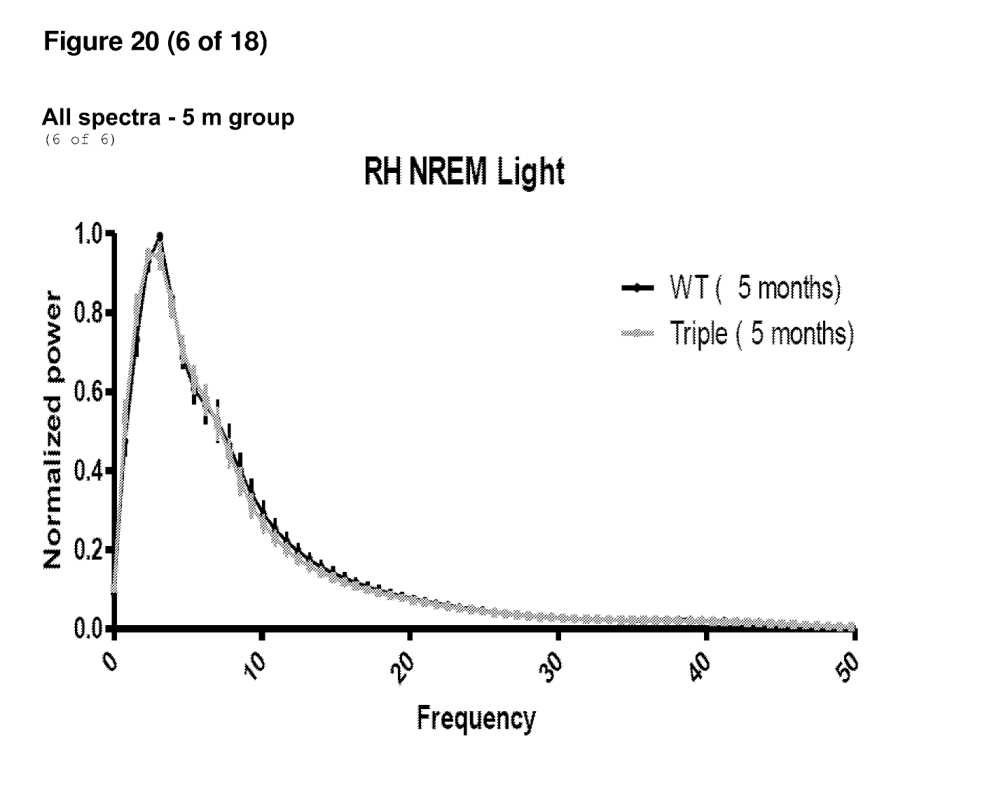

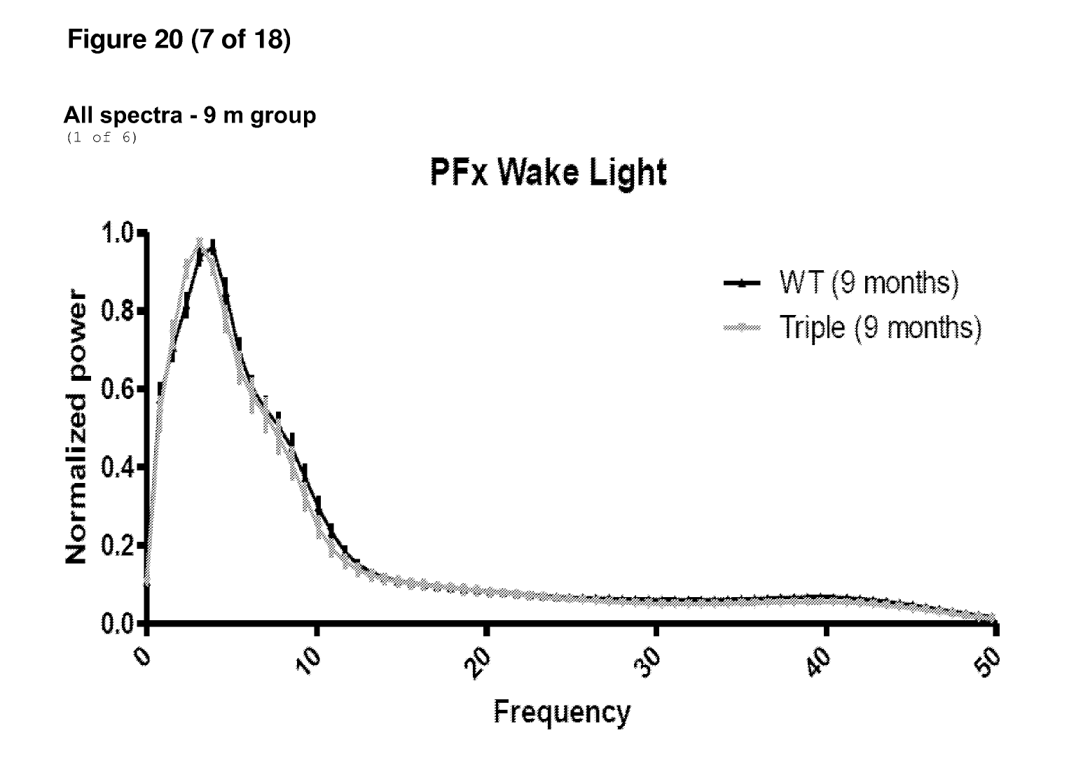

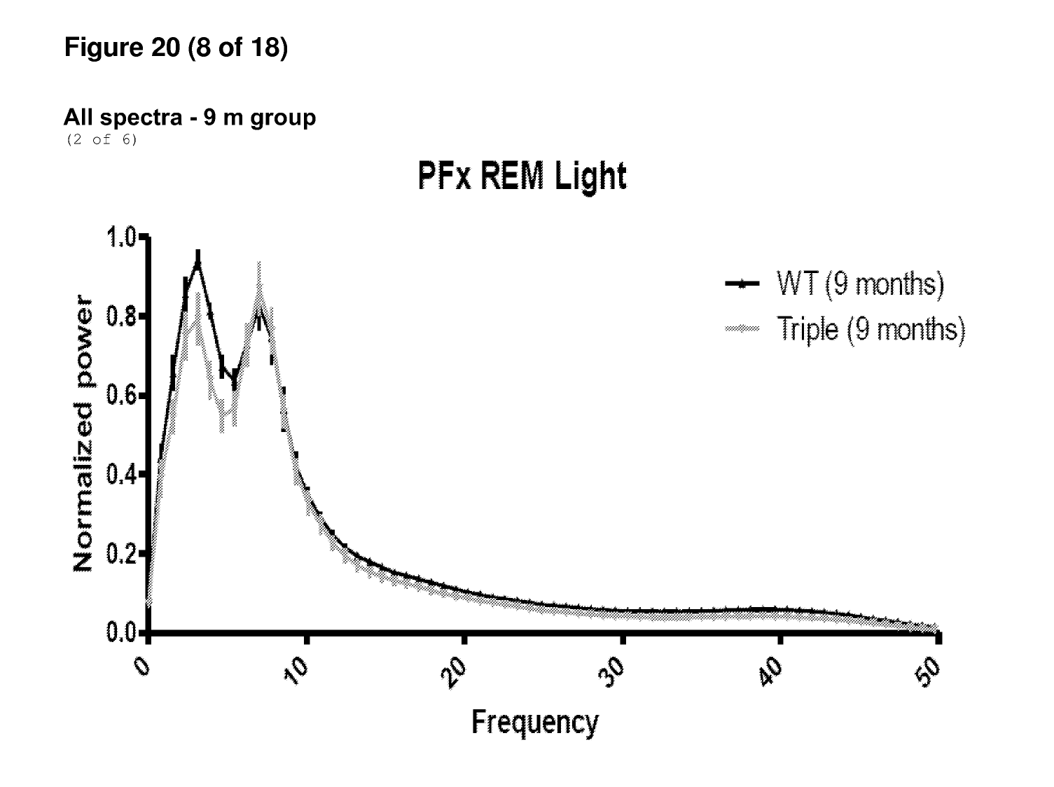

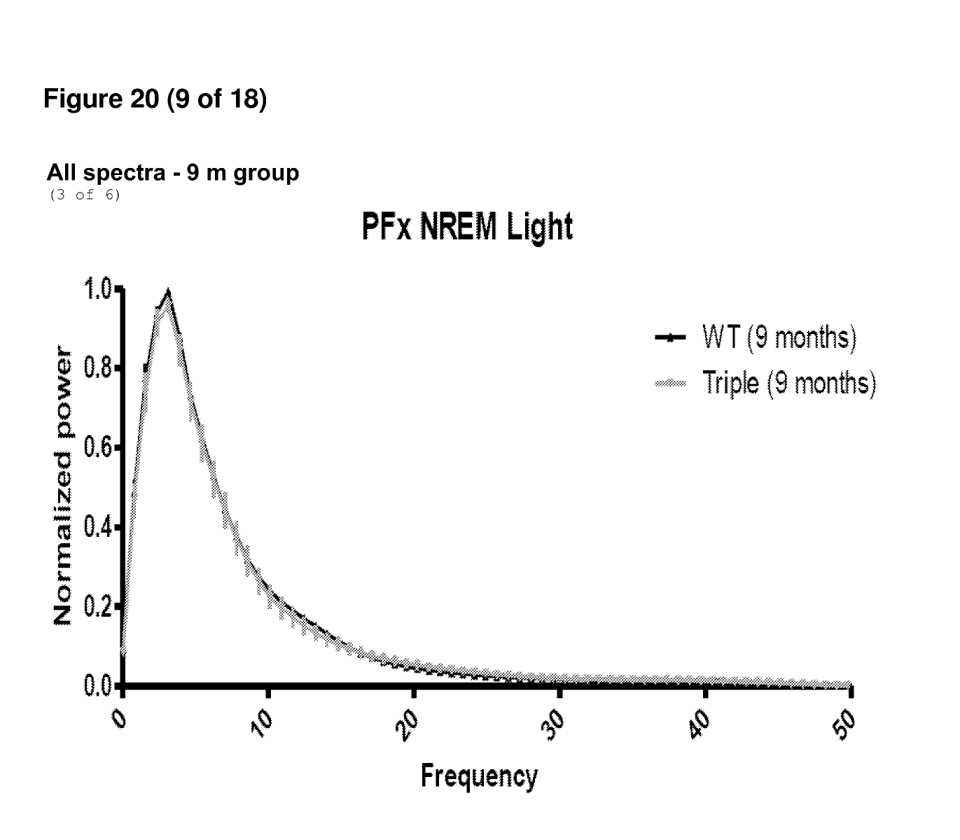

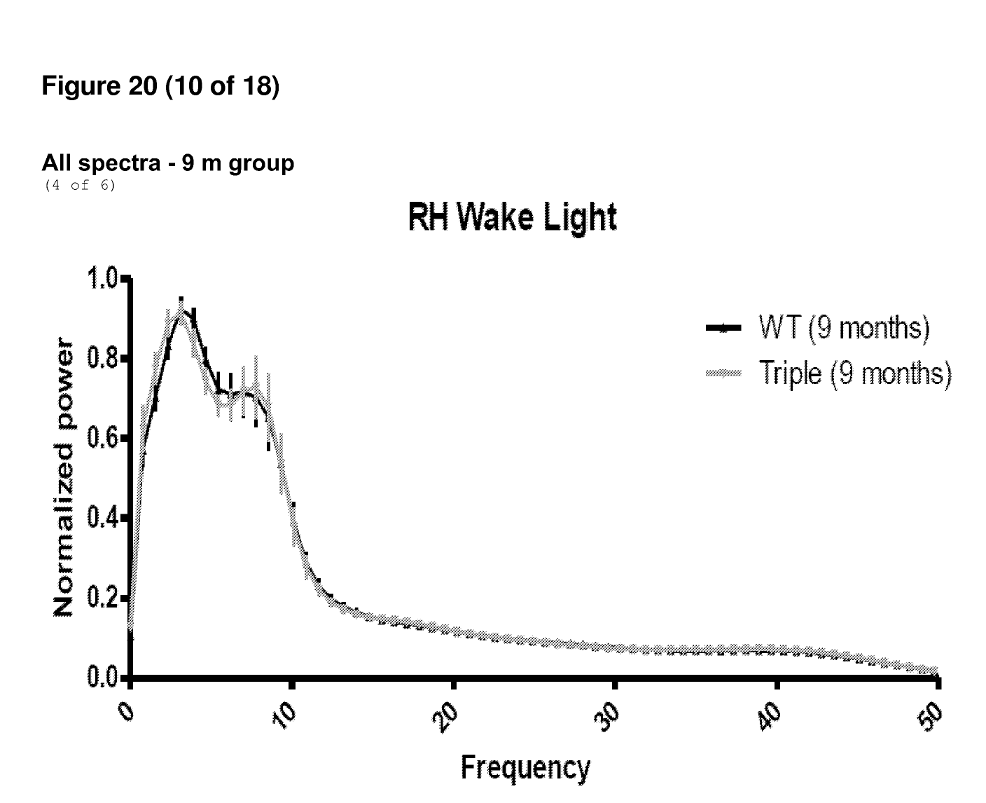

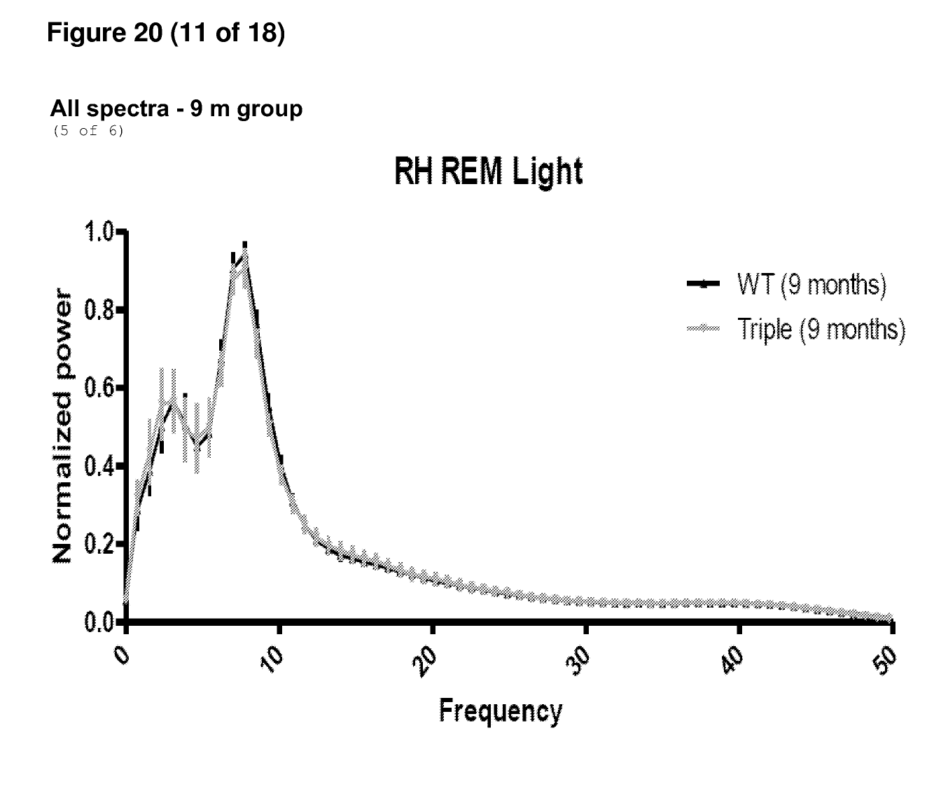

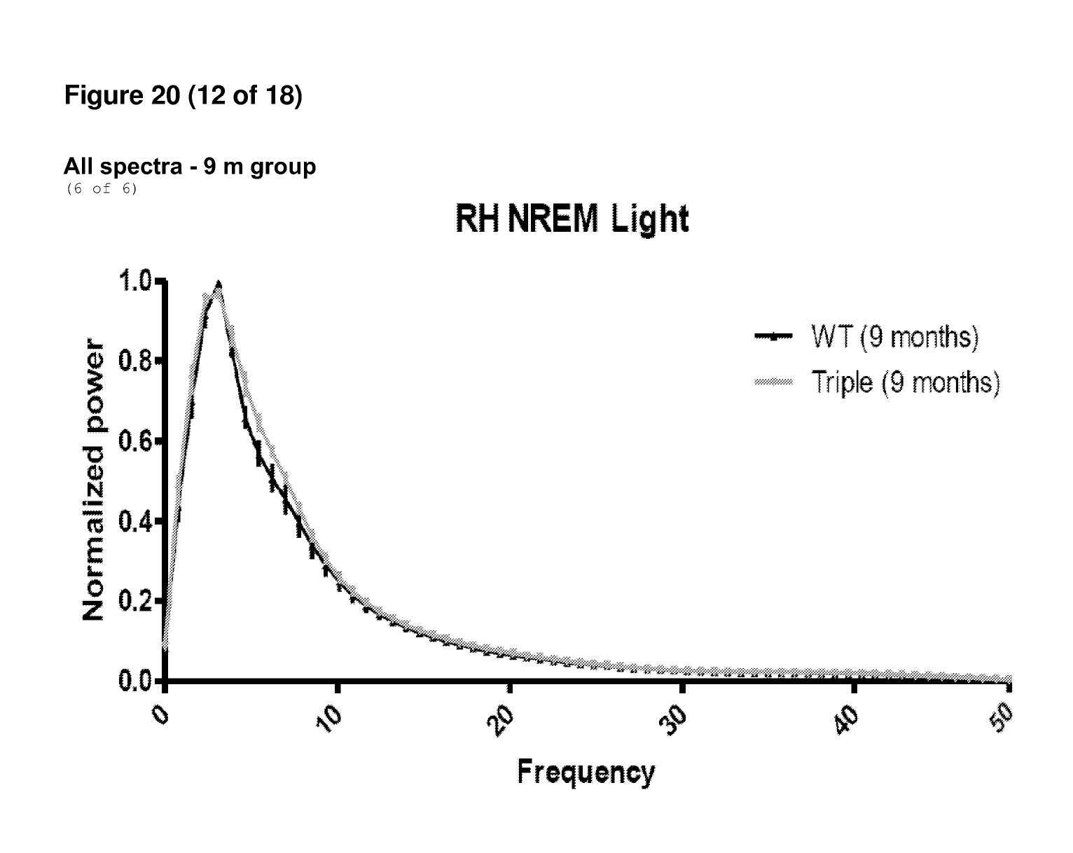

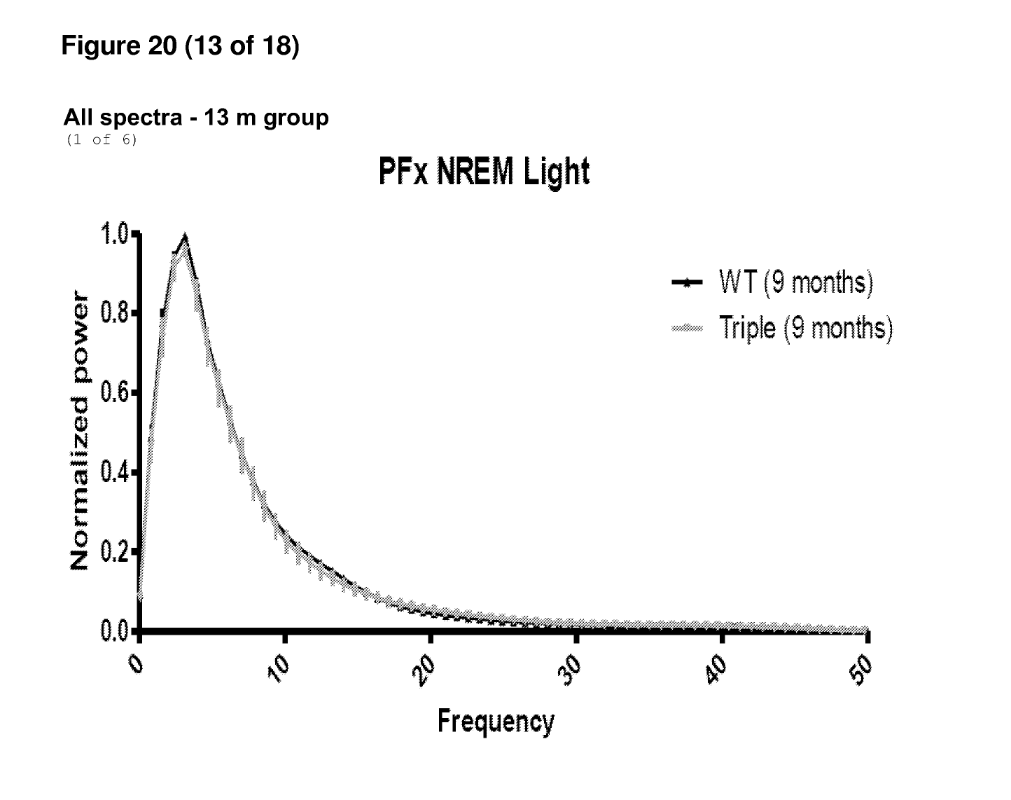

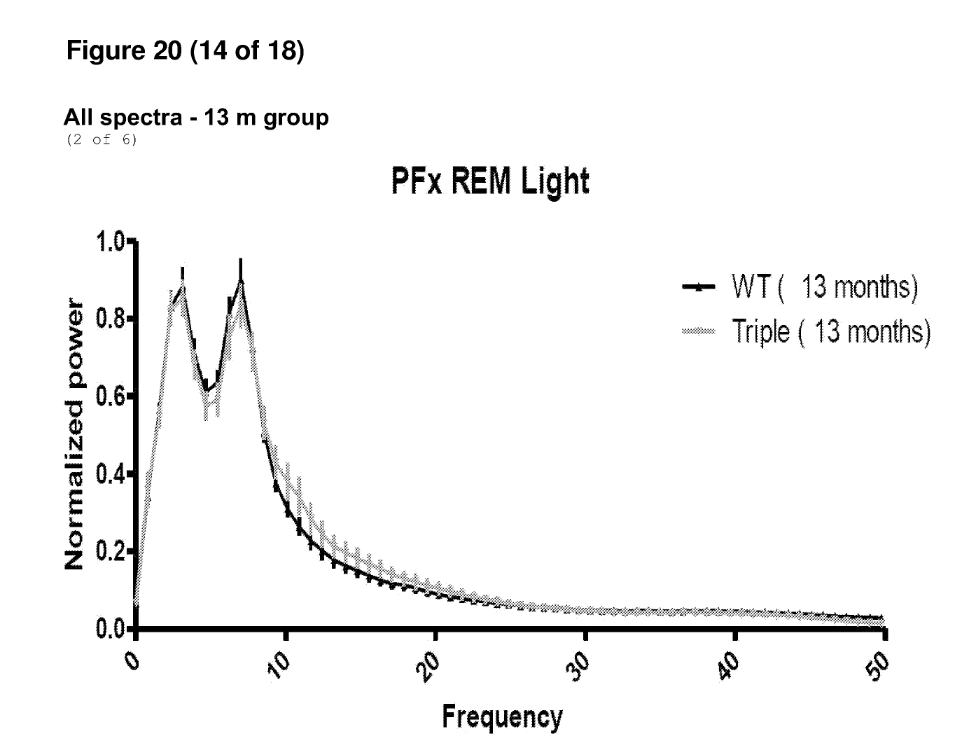

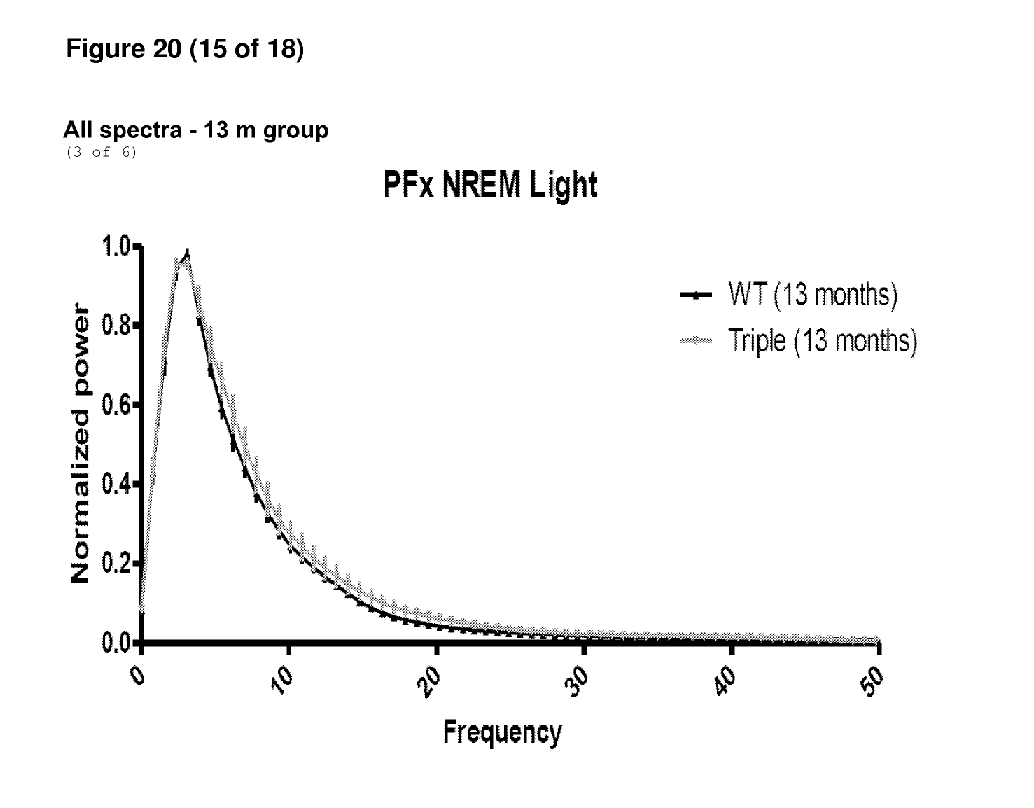

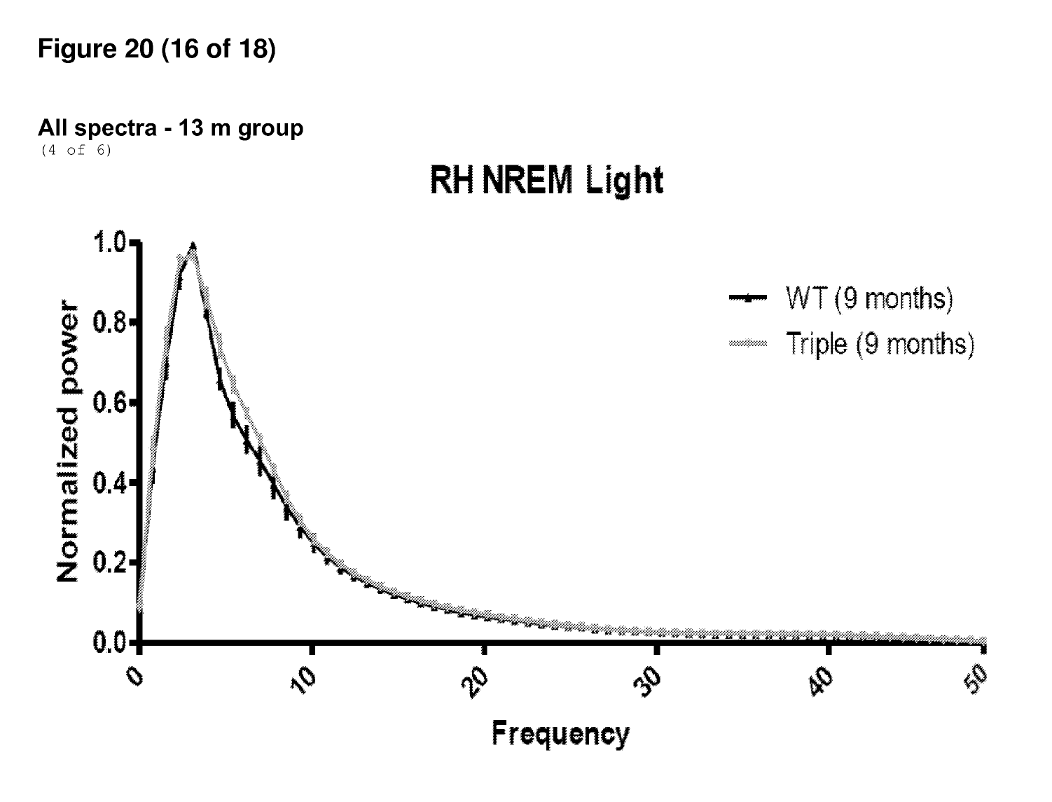

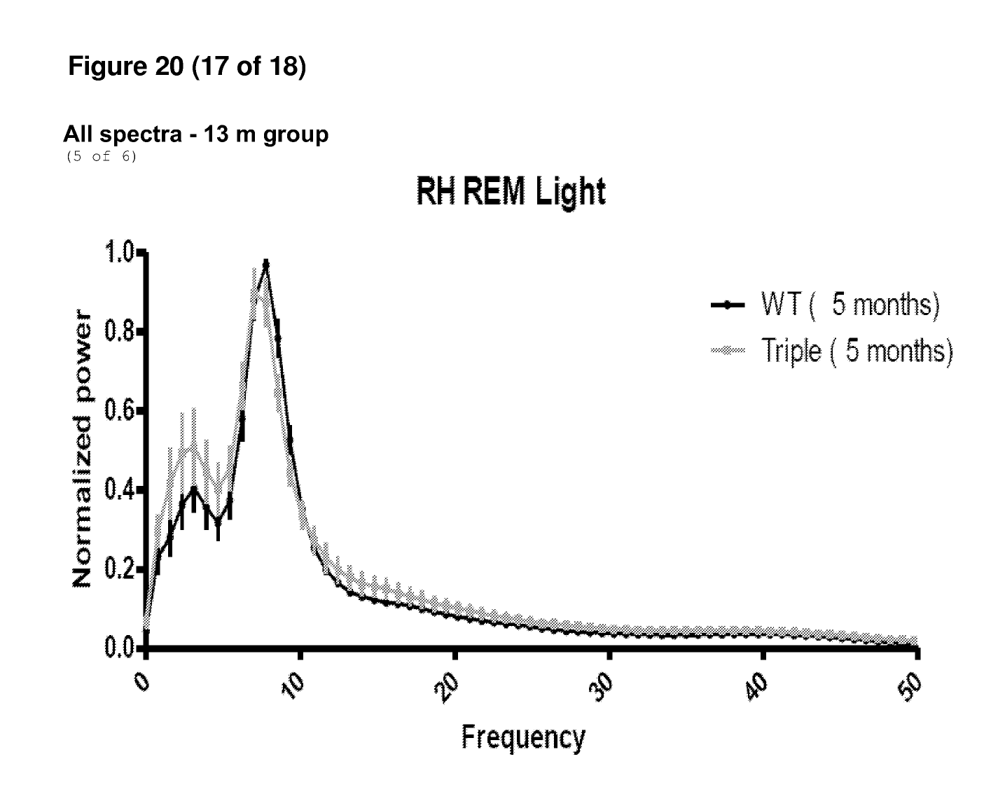

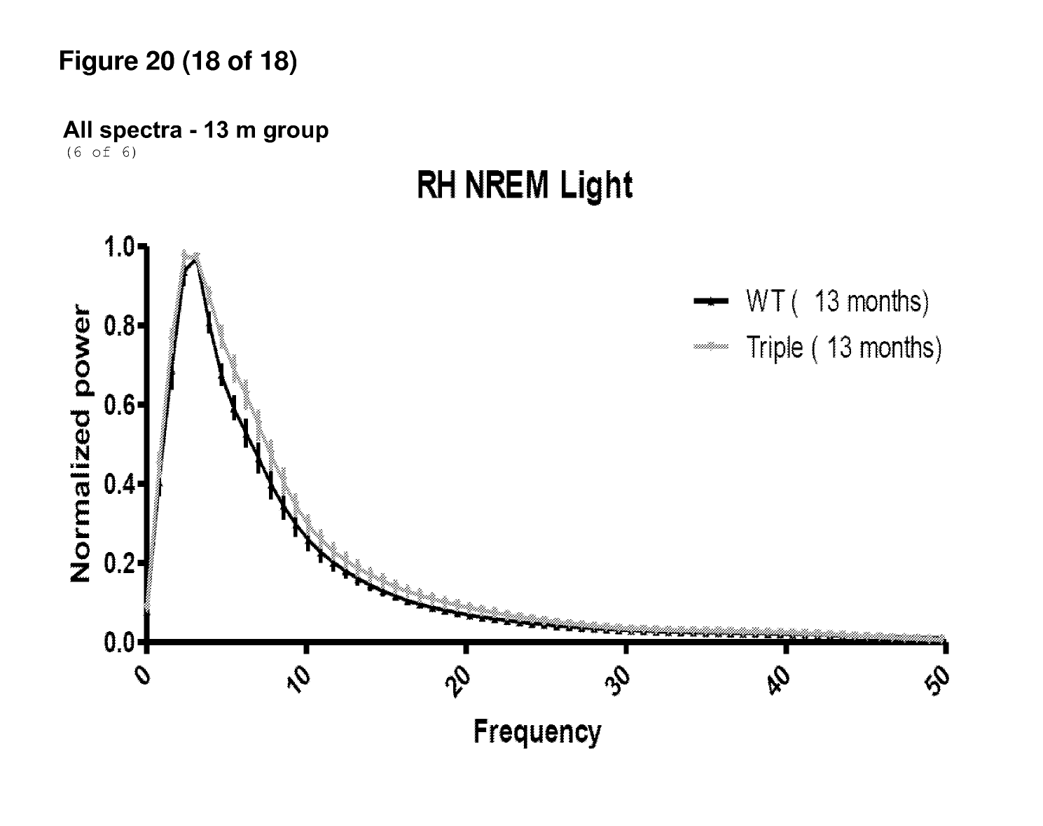

Power spectra analysis of wake, REM and NREM stages were observed separately in prefrontal cortex (PFx) and right hippocampus (RH). For clarity, specific components of the power spectra are illustrated (FIGS. 9 & 10) to visualize genotype- and age-specific spectral alterations, summarized in Tables 1 and 2 (see FIG. 20 for complete spectra). Genotype-specific alterations were observed in several frequency bands, with changes pronounced in the PFx and RH NREM power spectra. A genotype effect [F (1,126)=2.5 p<0.001] as well as an interaction (age×frequency) [F (6,126)=2.5 p<0.001] was noticed in the delta frequency band during wakefulness and NREM of 5 months old animals (FIG. 9A-D). The PFx gamma frequency band most consistently exhibited genotype effects in all vigilance stages and age groups (see FIG. 9, C,G,K and Tab. 1), indicating that gamma frequencies were particularly affected by the genotype. REM PFx EEG spectra from 5, 9 and 13 months age group presented a significant genotype effect in the alpha and beta range (FIG. 9 B,F,J), while RH NREM power spectra showed a significant genotype effect at the 9 months [(F(1,1170)=17.22 p<0.001] and 13 month [(F(64,1105)=46.6 p<0.001] only, but not at 5 months (FIG. 9 D,H,L). The pronounced power spectra data changes during NREM found in PLB1 Triples at 13 months in the RH may correspond to the phenotype of age-dependent increase in short NREM episodes (see above).

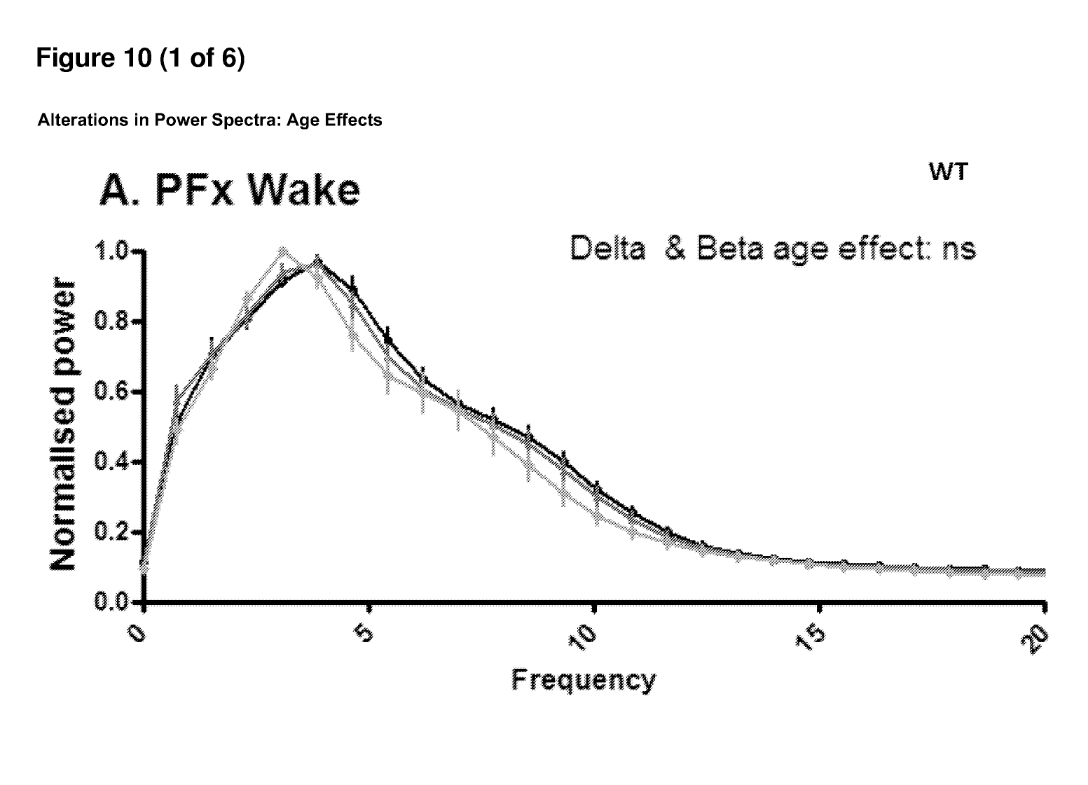

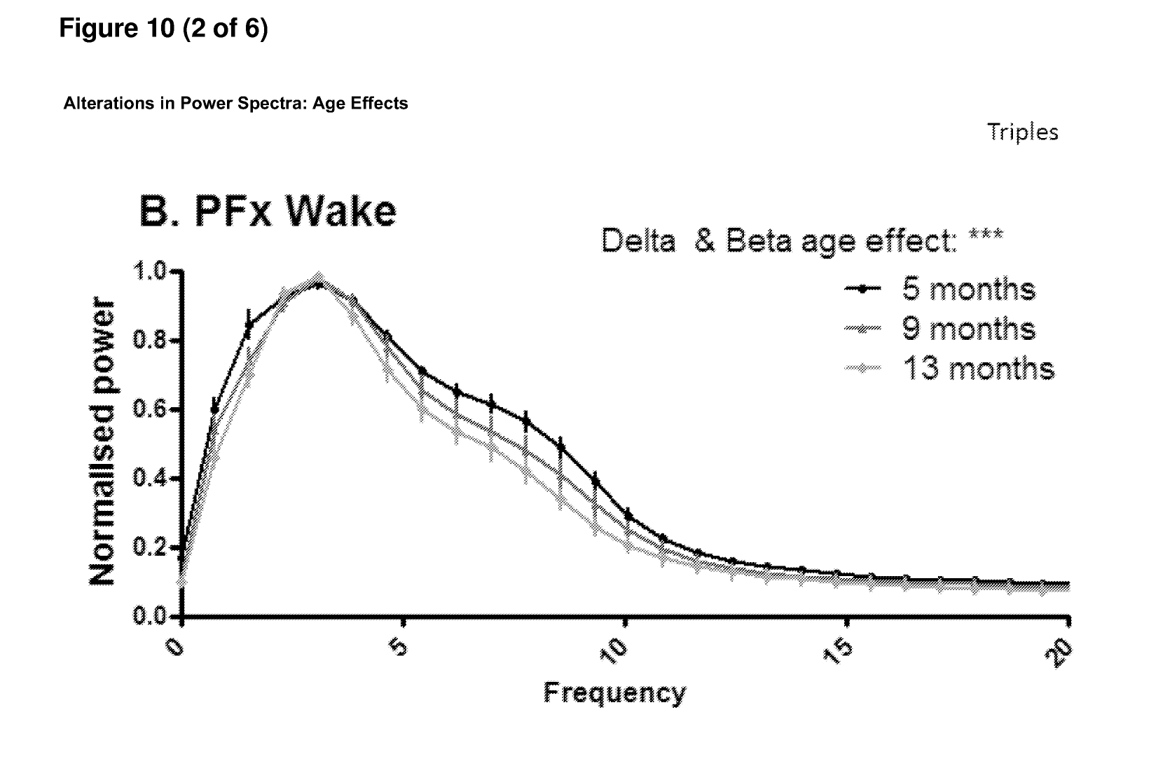

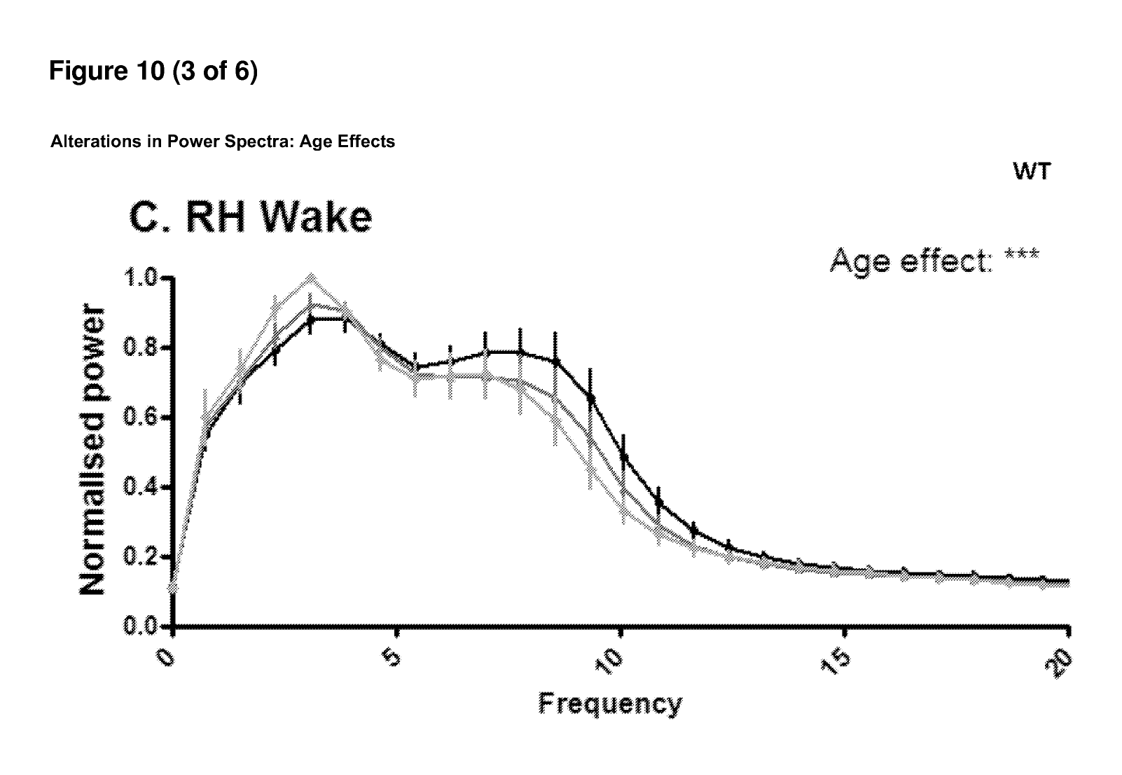

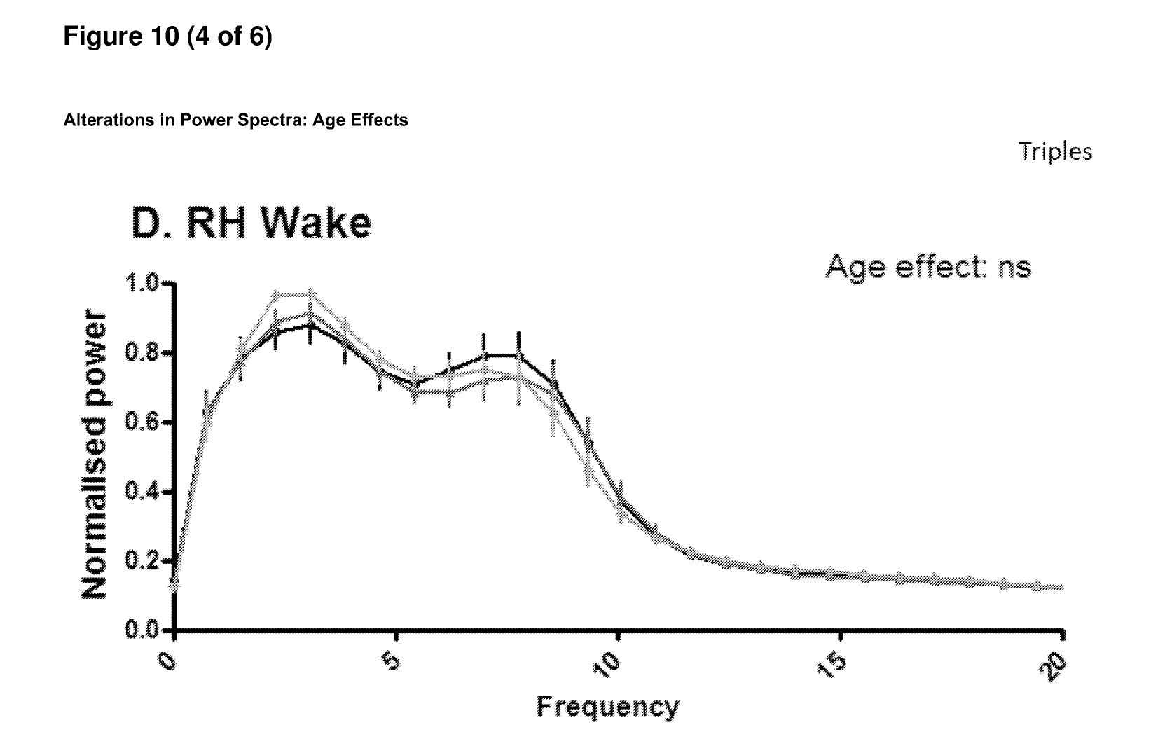

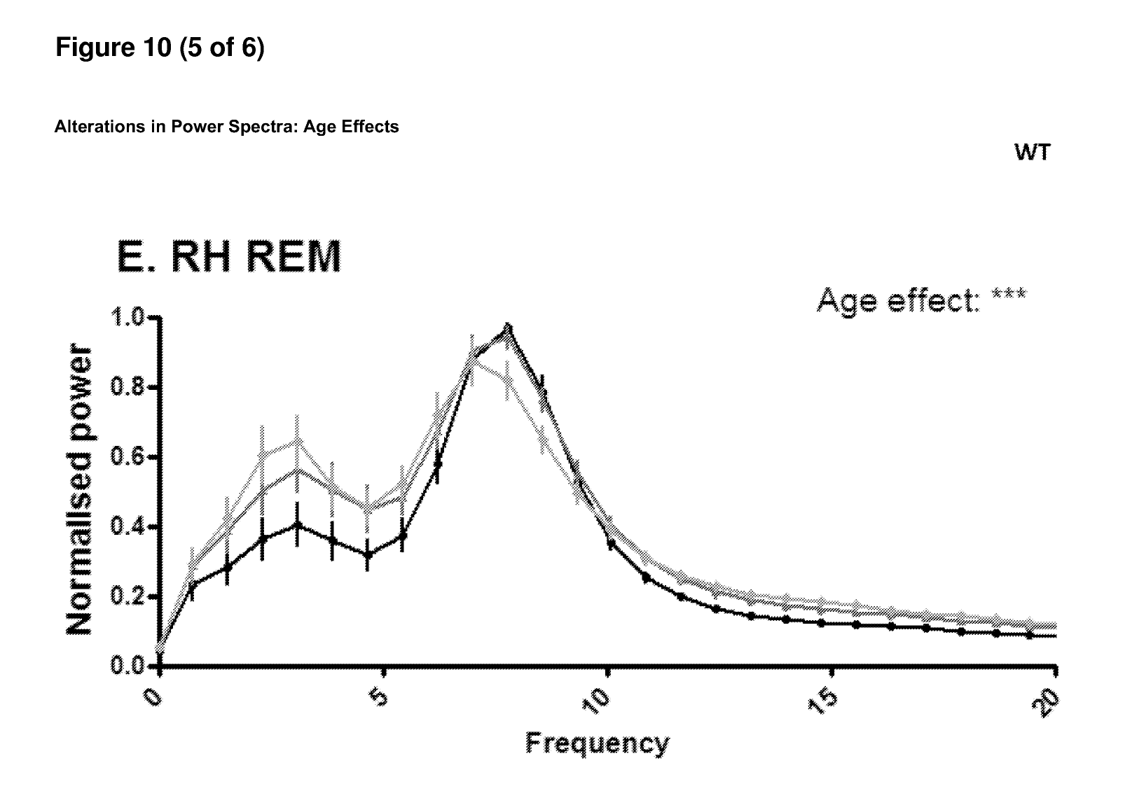

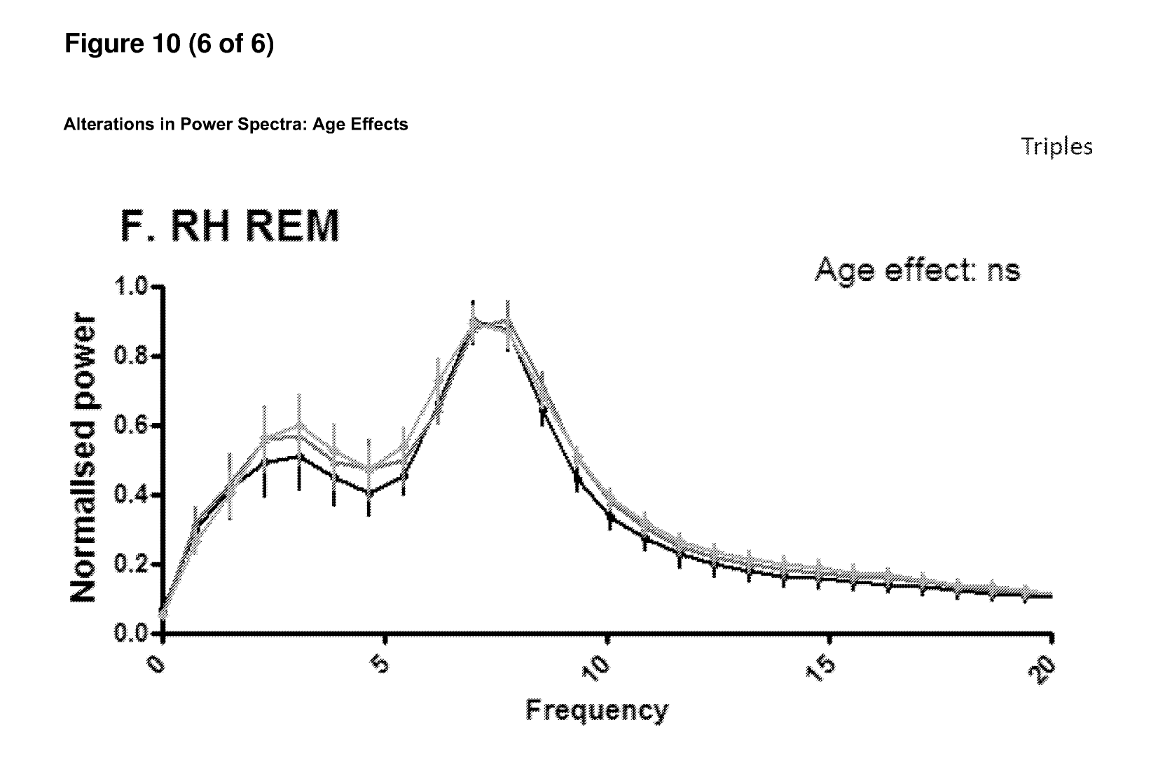

Additionally, the PFx delta band of PLB1 Triples [(F(2,189)=10.07 p<0.001] but not WTs showed a significant age-effect during wakefulness (FIG. 10B and Tab. 2), while conversely, in REM WT but not Triples [(F(2,189)=6 p<0.01] depicted significant age effect in the theta band (FIG. 10E). In agreement with data shown in Table 2, WTs presented most of the aging related alterations in RH power spectra during wakefulness and REM. The RH power spectra exhibited a significant age effect in WT during wakefulness [(F(2,1625)=5.62 p<0.01] (FIG. 10 C) and REM [(F(2,1625)=40.83 p<0.001] (FIG. 10E), while Triples did not show such overall spectral alterations with age (FIG. 10D&F).

Overall, the PFx EEG delta range during wakefulness and REM appears to be an indicator of early genotype-specific changes (at 5 months), while severe genotype-specific changes are identified at 13 months in NREM spectra of PFx (theta and gamma) and RH (delat & theta).

### 7. Learning & Memory

7.1. Object Recognition

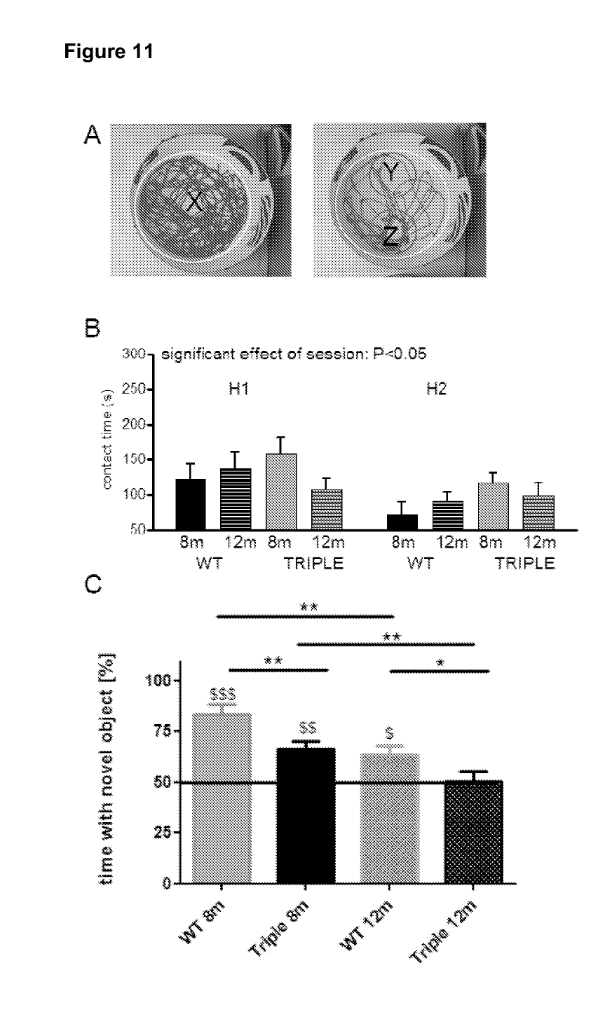

PLB1 animals were tested in an object recognition paradigm at 8 and 12 months of age (FIG. 11). During the two habituation sessions, both genotypes and age groups equally explored the object, with the contact time overall lower in the second habituation session (P<0.05), suggesting familiarity and habituation to the object (FIG. 11B). When a novel object was presented in the following session (object novelty), WT at both 8 and 12 months of age spent significantly more time (and above chance) exploring this object cf. the familiar object, while PLB1 Triples only preferred the novel object at 8 month (FIG. 11C). Furthermore, while an age effect was observed in both WT and Triples (P′ s<0.01), preference for the novel object was at the same level in Triples at 8 m same level as in WT at 12 m. Thus, WT and Triple differed significantly in both age groups (8 month: P<0.01; 12 month: P<0.05).

7.2. Social Interaction & Recognition

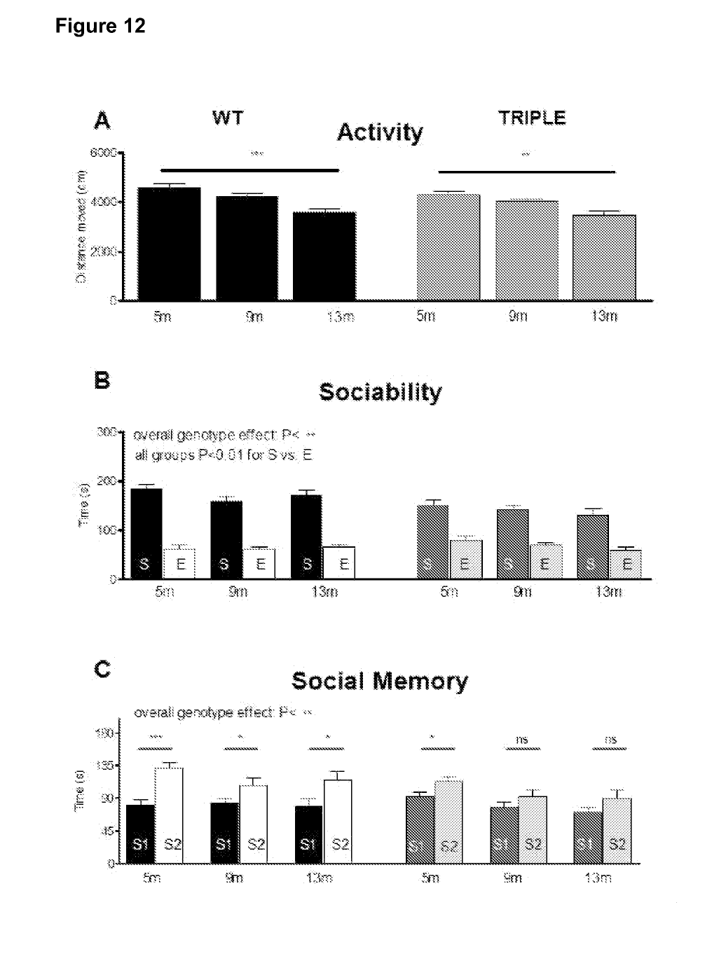

In addition to the inanimate object recognition paradigm, we also used a social recognition task to test social behaviour and memory in PLB1 mice. During habituation, animals generally visited all compartments of the arena. Occasionally, animals that did not show such exploration were excluded from the study. Activity analysed during the habituation phase (FIG. 12A) suggested an age-dependent, but no genotype-dependent, decline of activity, with 13-month old animals moving significantly less compared to 5-month old animals of both genotype (WT: P<0.001, Triple: P<0.01). During sociability, all groups of animals preferred the cylinder with the social stimulus over the empty container (all P′ s<0.01; FIG. 12B). However, a 2-way ANOVA confirmed a general genotype effect (F(1,220)=9.6), as PLB1 Triples showed overall less interest in S1 (time with S1: P<0.01).

In the memory phase of the paradigm (FIG. 12C), an overall comparison of time spent with novel stranger 2 (S2) also revealed a main overall effect of genotype (F(1,220)=10.3, P<0.01), but no age effect or interaction (F<1). Thus, Triple spent significantly less time interacting with S2 compared to WT (P<0.01). Paired comparison (stranger 1 vs. stranger 2) suggested that WT animals spent significantly more time with the novel stranger (S2) at all ages, with the greatest discrimination significance at 5 m (P<0.001 cf. P<0.05 at 9 and 13 m). Interestingly, PLB1 Triples only showed significant discrimination at 5 m of age (P<0.05).

Therefore, PLB1 Triple animals present with reduced interest in social stimuli, and do not show memory for a familiar compared to a novel stranger from 9 months of age.

### 9. PET

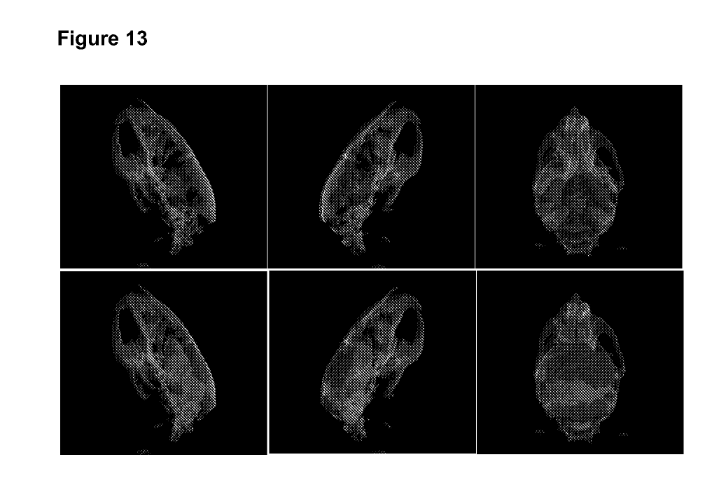

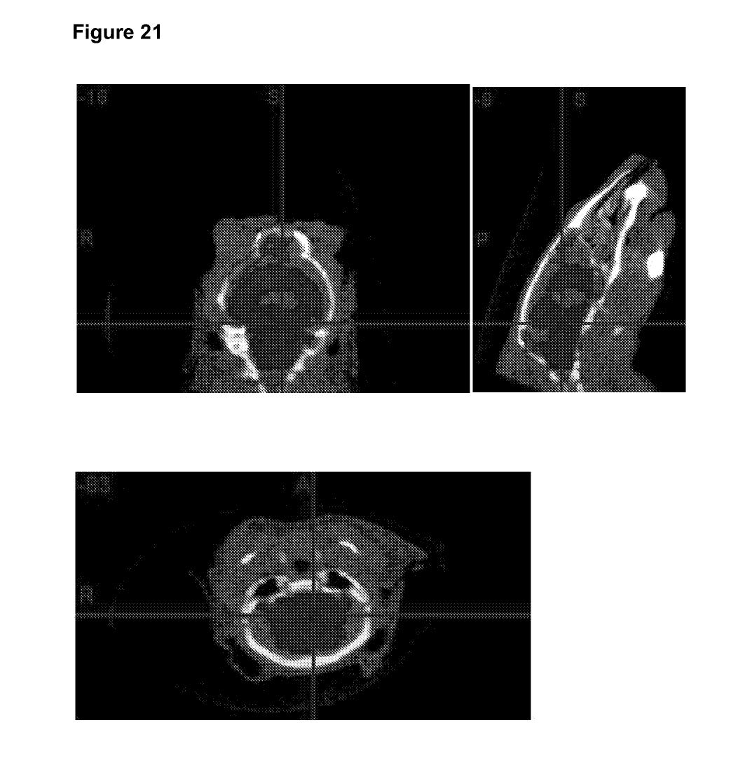

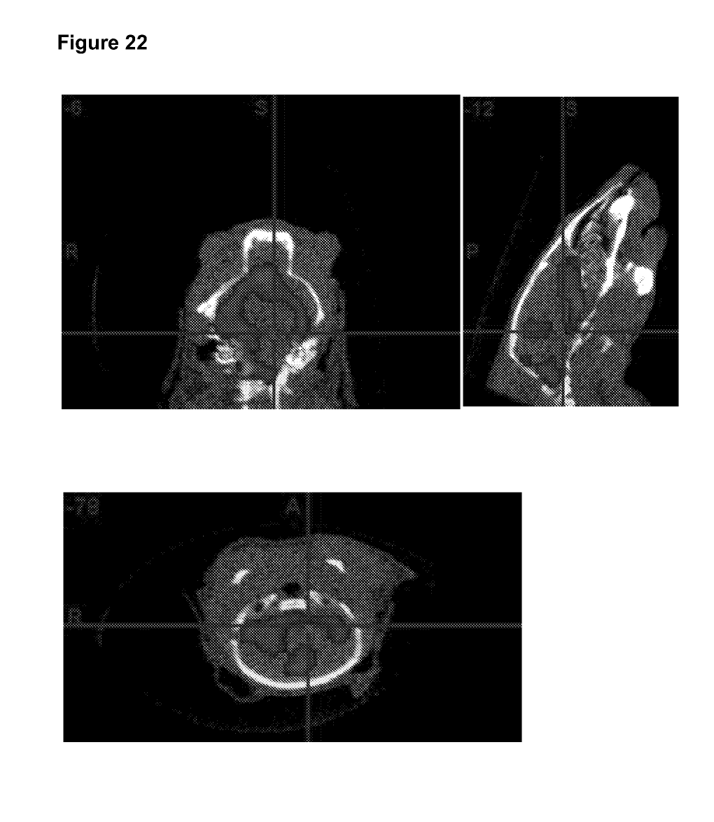

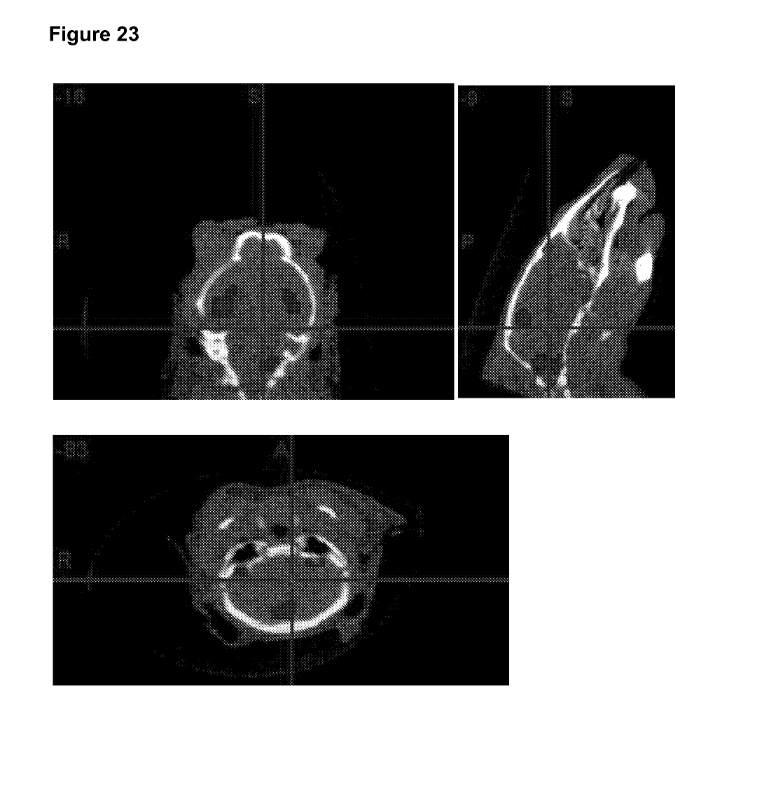

FIG. 13 shows projections through a 3D rendered object (for slices at various locations, see FIGS. 21-24). Areas of statistically significant (p<0.05) decrease in metabolism between 5 month (top row) and 15 month old (bottom row) PLB1 Triples vs. WT are shown in blue and areas of statistically significant increase (p<0.05) in metabolism are shown in red. Areas of increased metabolism at the front of the brain are likely to be artefacts caused by the sharp bend of the skull, as areas close to (or in this case seeming to overlap with) the skull can be caused by small differences in the registration of the images from different animals as there is a considerable change in signal between the brain and the skull.

At 5 months of age, no areas of increase were seen in PLB1 Triples relative to WT.

Large areas of decreased metabolism were observed most strikingly in the forebrain (hippocampal regions and adjunct limbic structures), and some additional areas in the dorsal midbrain and brainstem.

At 15 months, a wide-ranging increase in metabolism was found, with only some dorsal cortical areas showing reduced metabolism, and the rostral pole of the brain remaining unaltered. Thus, it may be the case that PLB1 triple are prematurely aged at 5 months cf. WT, followed by increased metabolic activity as a means to compensate for a progressive deficit.

### Tables

TABLE 1

| | | EEG during Light Phase | | | | | | | | |
|---|---|---|---|---|---|---|---|---|---|---|
| | | Wake | | | REM | | | NREM | | |
| | | 5 | 9 | 13 | 5 | 9 | 13 | 5 | 9 | 13 |
| | | months | months | months | months | months | months | months | months | months |
| PFx | Delta | int **/ | NS | NS | NS | gen *** | NS | int */ | NS | NS |
| | | gen ** | | | | | | gen * | | |
| | Theta | NS | NS | gen * | gen ** | NS | NS | NS | NS | gen * |
| | Alpha | NS | gen* | NS | gen *** | gen * | gen * | NS | NS | NS |
| | Beta | NS | NS | NS | gen *** | gen *** | gen * | NS | NS | gen ** |
| | Gamma | gen *** | gen *** | gen *** | NS | gen *** | NS | gen *** | gen * | gen *** |
| | All freq | int ***/ | gen *** | gen *** | int **/ | gen *** | NS | int */ | NS | gen *** |
| | | gen ** | | | gen ** | | | gen ** | | |
| RH | Delta | NS | NS | NS | gen ** | NS | NS | NS | gen * | gen ** |
| | Theta | NS | NS | NS | NS | NS | NS | NS | gen ** | gen *** |
| | Alpha | gen *** | NS | NS | NS | NS | NS | NS | NS | gen * |
| | Beta | NS | NS | NS | gen ** | NS | NS | NS | NS | gen ** |
| | Gamma | gen * | NS | NS | gen *** | NS | gen *** | NS | gen * | gen *** |
| | All freq | NS | NS | NS | gen *** | NS | NS | NS | gen *** | gen *** |

TABLE 2

| | | EEG during Light Phase | | | | | |
|---|---|---|---|---|---|---|---|
| | | Wake | | REM | | NREM | |
| | | WT | triple | WT | triple | WT | triple |
| PFx | Delta | NS | Age*** | Age** | NS | Age** | Age** |
| | range | | | | | | |
| | Theta | Age* | Age*** | NS | NS | NS | NS |
| | Alpha | Age* | Age*** | Age*** | Age** | NS | Age* |
| | Beta | NS | Age** | Age*** | Age** | Age** | Age* |
| | Gamma | Age*** | Age*** | Age*** | Age*** | Age*** | Age** |
| | All | Age*** | Int***/ | Age*** | Age*** | Age* | Age*** |
| | freq | | Age*** | | | | |
| RH | Delta | NS | NS | Age*** | NS | NS | NS |
| | range | | | | | | |
| | Theta | Age* | NS | NS | NS | Age* | Age* |
| | Alpha | Age** | NS | Age*** | NS | Age* | Age** |
| | Beta | NS | NS | Age*** | NS | Age** | Age*** |
| | Gamma | Age** | Age*** | Age*** | NS | Age*** | Age*** |
| | All | Age** | NS | Int***/ | NS | Age*** | Age*** |
| | freq | | | Age*** | | | |

### REFERENCES

LaFerla F M & Oddo S. Alzheimer's disease: Abeta, tau and synaptic dysfunction. Trends Mol. Med. 11, 170-6, 2005.
Nordberg, A.: PET imaging of amyloid in Alzheimer's disease. Lancet Neurol. 2004, 3:519-27, 2004.
Oddo S et al. Triple-transgenic model of Alzheimer's disease with plaques and tangles: intracellular Abeta and synaptic dysfunction. Neuron; 39, 409-21, 2003.
Good, M., Hale, G. (2005). Impaired visuospatial recognition memory but normal object novelty detection and relative familiarity judgments in adult mice expressing the APPswe Alzheimer's disease mutation. Behav Neurosci. 119(4):884-91.
Wang Y C, Seidel J, Tsui B M W, Vaquero J J & Pomper M G “Performance evaluation of the GE healthcare eXplore VISTA dual-ring small-animal PET scanner” JOURNAL OF NUCLEAR MEDICINE, Vol. 47 (11) pp 1891-1900, 2006
Rorden, C. & Brett, M. “Stereotaxic display of brain lesions” Behavioural Neurology, vol. 12, 191-200, 2000
Borchelt D R, Ratovitski T, van Lare J, Lee M K, Gonzales V, Jenkins N A, Copeland N G, Price D L, Sisodia S S. Accelerated amyloid deposition in the brains of transgenic mice coexpressing mutant presenilin 1 and amyloid precursor proteins. Neuron. 1997 October; 19(4):939-45.
B. Dreyer, W. Anderson, H. Johnson, M. O'Callaghan, S. Seo, D.-Y. Choi, G. Riedel & B. Platt: Memantine acts as a cholinergic stimulant in the mouse hippocampus. J. Alzheimer's Disease 12, 319-333, 2007.
Blennow, K. & Hampel, H. CSF markers for incipient Alzheimer's disease. Lancet Neurol. 2, 605-613 (2003).
Braak, E., Braak, H. & Mandelkow, E. M. A sequence of cytoskeleton changes related to the formation of neurofibrillary tangles and neuropil threads. Acta Neuropathol. 87, 554-567 (1994).
Deters, N., Ittner, L. M. & Gotz, J. Divergent phosphorylation pattern of tau in P301 L tau transgenic mice. Eur. J. Neurosci. 28, 137-147 (2008).
Giannakopoulos, P. et al. Assessing the cognitive impact of Alzheimer disease' pathology and vascular burden in the aging brain: the Geneva experience. Acta Neuropathol. 113, 1-12 (2007).
Noda-Saita, K. et al. Exclusive association and simultaneous appearance of congophilic plaques and AT8-positive dystrophic neurites in Tg2576 mice suggest a mechanism of senile plaque formation and progression of neuritic dystrophy in Alzheimer's disease. Acta Neuropathol. 108, 435-442 (2004).
Deacon R M, Koros E, Bornemann K D, Rawlins J N. Aged Tg2576 mice are impaired on social memory and open field habituation tests. Behav Brain Res. 2009 11; 197:466-8.
Gao X M, Elmer G I, Adams-Huet B, Tamminga C A. Social memory in mice: Disruption with an NMDA antagonist and attenuation with antipsychotic drugs. Pharmacol Biochem Behav. 2009; 92:236-42.
Kogan J H, Frankland P W, Silva A J. Long-term memory underlying hippocampus-dependent social recognition in mice. Hippocampus. 2000; 10:47-56.
Lee Y S, Silva A J. The molecular and cellular biology of enhanced cognition. Nat Rev Neurosci. 2009; 10:126-40.
Nadler J J, Moy S S, Dold G, Trang D, Simmons N, Perez A, Young N B, Barbaro R P, Piven J, Magnuson T R, Crawley J N. Automated apparatus for quantitation of social approach behaviors in mice. Genes Brain Behav. 2004; 3:303-14.
Wang et al. (J Nucl Med 2006; 47:1891-1900).
Oakley H, Cole S L, Logan S, Maus E, Shao P, Craft J, Guillozet-Bongaarts A, Ohno M, Disterhoft J, Van Eldik L, Berry R, Vassar R, Intraneuronal beta-amyloid aggregates, neurodegeneration, and neuron loss in transgenic mice with five familial Alzheimer's disease mutations: potential factors in amyloid plaque formation, J. Neurosci. 2006 Oct. 4; 26(40):10129-40.

1. A transgenic rodent which includes within a plurality of its cells a nucleic acid comprising:
(1) a mutated APP polynucleotide sequence flanked by a first set of excision sequences, and
(2) a mutated tau polynucleotide sequence flanked by a second set of excision sequences,
wherein (1) and (2) are operably linked to the same promoter sequence at a single locus.

2. The transgenic rodent of claim 1, wherein the first set of excision sequences comprises loxP sequences or FRT sequences.

3. The transgenic rodent of claim 1, wherein the second set of excision sequences comprises loxP sequences or FRT sequences, wherein the first and second set of excision sequences are different from each other.

4. The transgenic rodent of claim 1, wherein the mutated APP polynucleotide sequence encodes a polypeptide comprising one or more of the following mutations: K670N; M671L; and V717I, wherein optionally the mutated APP polynucleotide sequence comprises SEQ ID NO:1.

5. The transgenic rodent of claim 1, wherein the mutated tau polynucleotide sequence encodes a polypeptide comprising one or more of the following mutations: P301L and R406W, wherein optionally the mutated tau polynucleotide sequence comprises SEQ ID NO:2.

6. The transgenic rodent of claim 1, wherein the promoter sequence is a CamK2 promoter.

7. The transgenic rodent of claim 1, wherein the nucleic acid further comprises a marker gene, wherein optionally the marker gene is the neomycin resistance gene.

8. The transgenic rodent of claim 1, wherein the nucleic acid further comprises an internal ribosome entry site positioned between the APP and tau polynucleotide sequences.

9. The transgenic rodent of claim 1, wherein said polynucleotide sequences are heterologous with respect to the transgenic rodent.

10. The transgenic rodent of claim 1, wherein the single locus is the HPRT locus.

11. The transgenic rodent of claim 1, wherein the rodent is hemizygous, heterozygous or homozygous with respect to said nucleic acid.

12. The transgenic rodent of claim 1, wherein the nucleic acid is present in the transgenic rodent at one copy per cell.

13. The transgenic rodent of claim 1, further including in said plurality of cells a presenilin polynucleotide sequence.

14. The transgenic rodent of claim 1 having one or more of the following phenotypes: intracellular and extracellular amyloid deposits, impaired synaptic transmission, reduced paired pulse facilitation (PPF), deficit in LTP, reduced activity in dark phase, spending more time awake, sleep disturbance and sleep fragmentation, reduced REM and NREM sleep, cognitive deficits, altered memory, premature aging, and altered metabolism in the brain.

15. A somatic cell or tissue sample of the transgenic rodent as claimed in claim 1.

16. A gamete of the transgenic rodent as claimed in claim 1.

17. A nucleic acid comprising a
(1) a mutated APP polynucleotide sequence flanked by a first set of excision sequences, and
(2) a mutated tau polynucleotide sequence flanked by a second set of excision sequences
wherein (1) and (2) are operably linked to the same promoter sequence.

18. The nucleic acid of claim 17, wherein the first set of excision sequences comprises loxP sequences or FRT sequences.

19. The nucleic acid of claim 17, wherein the second set of excision sequences comprises loxP sequences or FRT sequences, wherein the first and second set of excision sequences are different from each other.

20. The nucleic acid of claim 17, wherein the mutated APP polynucleotide sequence encodes a polypeptide comprising one or more of the following mutations: K670N; M671L; and V717I, wherein optionally the mutated APP polynucleotide sequence comprises SEQ ID NO:1.

21. The nucleic acid of claim 17, wherein the mutated tau polynucleotide sequence encodes a polypeptide comprising one or more of the following mutations: P301L and R406W, wherein optionally the mutated tau polynucleotide sequence comprises SEQ ID NO:1.

22. The nucleic acid of claim 17, wherein the promoter sequence is a CamK2 promoter.

23. The nucleic acid of any one of claim 17, further comprising a marker gene, wherein optionally the marker gene is the neomycin resistance gene.

24. The nucleic acid of any one of claim 17, further comprising an internal ribosome entry site positioned between the APP and tau polynucleotide sequences.

25. A vector comprising the nucleic acid of claim 17.

26. A targeting vector comprising the nucleic acid of claim 17, further comprising a targeting sequence.

27. The targeting vector of claim 17, wherein the targeting sequence is a sequence targeting the HPRT locus.

28. A cell comprising the nucleic acid or vector of any one of claim 17.

29. The cell of claim 28, wherein the cell is a rodent embryonic stem cell.

30. A method of generating a transgenic rodent, the method comprising
(a) injecting an ES cell into a rodent blastocyst, the ES cell comprising the nucleic acid of claim 17,
(b) implanting said blastocyst into a surrogate female rodent,
(c) allowing the surrogate female rodent to produce offspring,
(d) screening the offspring for the introduction of said nucleic acid in the genome, and, optionally,
(e) crossing the offspring with a wildtype rodent of the same species and obtaining F1 offspring.

31. The method of claim 30, further comprising the steps of
(i) providing the offspring of the method of claim 30,
(ii) excising the APP or tau polynucleotide sequence,
and optionally (iii) obtaining resulting offspring after step (ii)
and optionally (iv) testing the resulting offspring for the excision of the APP or tau polynucleotide sequence, respectively.

32. The method of claim 30, further comprising the steps of
(i) providing the offspring of the method of claim 30,
(ii) crossing the offspring with a rodent capable of expressing a recombinase specific for the first or second set of recombination sites.

33. The method of claim 32, further comprising the steps of
(iii) obtaining the resulting offspring,
and optionally (iv) testing the resulting offspring for the excision of the APP or tau polynucleotide sequence, respectively.

34. The method of claim 30, further comprising the step of (i) crossing the F1 offspring or resulting offspring with another transgenic rodent, said other transgenic rodent including a mutant presenilin polynucleotide sequence.

35. The method of claim 34, further comprising the steps of
(ii) obtaining offspring,
and optionally (iii) testing the offspring of step (ii) for the presence of one or more of said APP, tau and presenilin polynucleotide sequences.

36. A transgenic rodent obtainable by the method of claim 30.

37. A method of modelling Alzheimer's disease by providing the transgenic rodent of claim 1 and monitoring changes in one or more of the phenotypes of the rodent.

38. A method of screening or assessing a compound suspected of having a therapeutic effect in relation to Alzheimer's disease, the method comprising:
(a) providing the transgenic rodent of claim 1,
(b) administering the compound to the rodent,
(c) monitoring changes in one or more of the phenotypes of the rodent, wherein, optionally, the phenotype monitored is selected from intracellular and extracellular amyloid deposits, impaired synaptic transmission, reduced paired pulse facilitation (PPF), deficit in LTP, reduced activity in dark phase, spending more time awake, sleep disturbance and sleep fragmentation, reduced REM and NREM sleep, cognitive deficits, altered memory, premature aging, and altered metabolism in the brain.

39. The transgenic rodent of claim 1 wherein the transgenic rodent is a mouse.

40. A system comprising
(1) providing a double or triple transgenic rodent generated by the methods of claim 30,
(2) providing an excised control rodent obtainable by the method of claim 31,
(3) comparing the phenotype of (1) with the phenotype of (2).


SEQUENCE LISTING (19 sequences)

SEQ ID NO: 1 - DNA, 2313 bp, Artificial sequence
  Synthetic sequence Double mutated APP derived from isoform a (APP770) - with Swedish mutation (K670N; M671L) and London mutation (V717I)
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaatctggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcatcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 2 - DNA, 2277 bp, Artificial sequence
  Synthetic sequence Tau coding sequence with P301L and R406W mutations (derived from NM_016835 sequence)
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaactccccactcctggtagctcagaccctctgatccaaccctccagccctgctgtgtgcccagagccaccttcctctcctaaacacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtctcgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccatggcatctcagcaatgtctcctccaccggcagcatcgacatggtagactcgccccagctcgccacgctagctgacgaggtgtctgcctccctggccaagcagggtttgtga

SEQ ID NO: 3 - DNA, 32 bp, Artificial sequence
  Synthetic sequence Forward primer GW496
acaattgcctgtgaatcaagttctagatctgg

SEQ ID NO: 4 - DNA, 29 bp, Artificial sequence
  Synthetic sequence Reverse primer GW497
ttcgtccagatcatcctgatcgacaagac

SEQ ID NO: 5 - DNA, 32 bp, Artificial sequence
  Synthetic sequence Hprt WT primer pair 1
tgtccttagaaaacacatatccagggtttagg

SEQ ID NO: 6 - DNA, 30 bp, Artificial sequence
  Synthetic sequence Hprt WT primer pair 2
ctggcttaaagacaacatctgggagaaaaa

SEQ ID NO: 7 - DNA, 21 bp, Artificial sequence
  Synthetic sequence Human APP primer (forward)
actggctgaagaaagtgacaa

SEQ ID NO: 8 - DNA, 22 bp, Artificial sequence
  Synthetic sequence Human APP primer (reverse)
atcaccatcctcatcgtcctcg

SEQ ID NO: 9 - DNA, 20 bp, Artificial sequence
  Synthetic sequence Human Tau primer (forward)
cacggacgctggcctgaaag

SEQ ID NO: 10 - DNA, 20 bp, Artificial sequence
  Synthetic sequence Human Tau primer (reverse)
ctgtggttccttctgggatc

SEQ ID NO: 11 - DNA, 20 bp, Artificial sequence
  Synthetic sequence gene mouse GAPDH primer (forward)
actttgtcaagctcatttcc

SEQ ID NO: 12 - DNA, 20 bp, Artificial sequence
  Synthetic sequence gene mouse GAPDH primer (reverse)
tgcagcgaactttattgatc

SEQ ID NO: 13 - protein, 42 aa, Homo sapiens [sequence omitted]

SEQ ID NO: 14 - protein, 42 aa, Rattus norvegicus
DAEFGHDSGFEVRHQKLVFFAEDVGSNKGAIIGLMVGGVVIA

SEQ ID NO: 15 - protein, 42 aa, Mus musculus; identical to SEQ ID NO: 14

SEQ ID NO: 16 - protein, 42 aa, Xenopus laevis
DSEYRHDTAYEVHHQKLVFFAEEVGSNKGAIIGLMVGGVVIA

SEQ ID NO: 17 - protein, 42 aa, Xenopus laevis
DSEYRHDAAYEVHHQKLVFFADEVGSNKGAIIGLMVGGVVIA

SEQ ID NO: 18 - DNA, 3648 bp, Homo sapiens
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 19 - DNA, 6762 bp, Homo sapiens
acggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaacacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcacccctgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtgggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcggcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa